(19) United States
(12) Patent Application Publication
(10) Pub. No.: US 2013/0017188 A1
(43) Pub. Date: Jan. 17, 2013

(54) MODULATION OF SGK1 EXPRESSION IN TH17 CELLS TO MODULATE TH17-MEDIATED IMMUNE RESPONSES

(71) Applicants: Vijay K. Kuchroo, Newton, MA (US); Hulin Jin, Boston, MA (US)

(72) Inventors: Vijay K. Kuchroo, Newton, MA (US); Hulin Jin, Boston, MA (US)

(73) Assignee: THE BRIGHAM AND WOMEN'S HOSPITAL, INC., Boston, MA (US)

(21) Appl. No.: 13/387,003

(22) PCT Filed: Jul. 30, 2010
(86) PCT No.: PCT/US10/43907

§ 371 Date: Jul. 6, 2012

Related U.S. Application Data
(60) Provisional application No. 61/230,376, filed on Jul. 31, 2009.

(51) Int. Cl. A61K 31/404; A61K 39/395; A61K 31/713; A61K 31/7105; A61P 37/00; A61P 25/00; A61P 29/00; A61P 17/06; A61P 19/02; A61P 19/04; A61P 5/16; A61P 1/00; A61P 1/18; A61P 3/10; A61P 27/02; A61P 1/04; C12N 5/0783; A61K 31/437

(52) U.S. Cl. 424/130.1; 514/415; 514/300; 514/44 A; 514/44 R; 435/377

(57) ABSTRACT

The inventors have made the surprising discovery that SGK1, a serine/threonine kinase previously described as being involved in regulation of cellular sodium homeostasis, has a novel and unexpected function in the differentiation and function of a specific subset of CD4 T cells, the TH17 lineage. Described herein are methods and compositions for modulation of TH17 cell differentiation, proliferation, and/or function that rely upon modulating the activity or expression of SGK1. Such methods and compositions are useful in the treatment of disorders including autoimmune diseases, chronic inflammatory conditions, infectious diseases, and cancer.

### CROSS REFERENCE TO RELATED APPLICATIONS

This application claims benefit under 35 U.S.C. §119(e) of U.S. Provisional Application No. 61/230,376 filed Jul. 31, 2009, the contents of which are herein incorporated by reference in its entirety.

### GOVERNMENT SUPPORT

This invention was made with Government Support under NIH Grant No. R37 NS030843. The Government has certain rights in the invention.

### FIELD OF THE INVENTION

The invention relates to compositions and methods for modulating TH17 responses.

### BACKGROUND

TH17 cells are a subset of CD4+ T cells that are abundant at mucosal interfaces, where they can contain infection with pathogenic bacteria and fungi (Weaver et al., 2007). TH17 cells produce IL-17A (also referred to as IL-17), IL-17F, and IL-22, cytokines involved in neutrophilia, tissue remodeling and repair, and production of antimicrobial proteins. TH17 cells differentiate in response to the STAT3-activating cytokines IL-6, IL-21, and IL-23, along with TGF-β and IL-1β (Korn et al., 2009).

SGK-1 (serum and glucocorticoid-regulated kinase-1) belongs to a family of serine/threonine kinases of which to date three members are known and are referred to as SGK-1, SGK-2 and SGK-3/SGKL/CISK. SGK-1 is expressed in virtually all tissues which have been tested to date (Gonzalez-Robayna et al., 1999; Waldegger et al., 1999; Alliston et al., 2000; Klingel et al., 2000; Lang et al., 2000; Loffing et al., 2001; Fillon et al., 2002; Warntges et al., 2002a;). A large number of stimuli have been shown to activate transcription of SGK-1, including mineralocorticoids, gonadotropins, osmotic, cell-volume and hypotonic changes, and 1,25(OH)₂D₃ (Brennan et al., 2000; Shigaev et al., 2000; Bhargava et al., 2001, Richards et al., 1995; Gonzalez-Robayna et al., 2000, Akutsu et al., 2001).

### SUMMARY OF THE INVENTION

The inventors have made the surprising discovery that SGK1, a serine/threonine kinase previously described as being involved in regulation of cellular sodium homeostasis, has a novel and unexpected function in the differentiation and function of a specific subset of CD4 T cells, the TH17 lineage. Described herein are methods and compositions for modulation of TH17 cell differentiation, proliferation, and/or function that rely upon modulating the activity or expression of SGK1.

Accordingly, in one aspect described herein are methods of inhibiting differentiation of a CD4⁺ T cell or a CD4⁺ T cell population into a TH17 cell or TH17 cell population. Such methods comprise contacting a CD4⁺ T cell or CD4⁺ T cell population with a serum and glucocorticoid-regulated kinase 1 (SGK1) inhibitor in an amount sufficient to inhibit TH17 cell differentiation. In some embodiments, the methods of inhibiting differentiation into a TH17 cell or TH17 cell population further comprise contacting the CD4⁺ T cell or CD4⁺ T cell population with an inhibitor or antagonist of one or more of the following molecules: TGF-β, IL-6, IL-21, IL-23, RORγt, RORα, STAT3, IRF4, AhR (aryl hydrocarbon receptor), and BATf.

In another aspect, described herein are methods of inhibiting a TH17 cell-mediated immune response in a subject in need thereof. Such methods comprise administering to a subject in need thereof a therapeutically effective amount of a serum and glucocorticoid-regulated kinase 1 (SGK1) inhibitor to inhibit a TH17 cell-mediated immune response. In some embodiments of these methods, the TH17 cell-mediated response being inhibited comprises expression or production of IL-17 by a TH17 cell. In some embodiments of these methods, the TH17 cell-mediated response being inhibited further comprises expression or production of one or more of IL-17F, IL-22, IL-26, IL-21, and TNF-α.

In some embodiments of these methods, the TH17 cell-mediated response being inhibited comprises inhibition of proliferation of or expansion of a TH17 cell. In some embodiments of these methods, the TH17 cell-mediated response being inhibited comprises trafficking of a TH17 cell.

In some embodiments of these methods, the subject in need of inhibition of a TH17-mediated immune response has a TH17-mediated disorder. In some such embodiments, the TH17-mediated disorder is an autoimmune disease or a chronic inflammatory disease. In some embodiments, the autoimmune disease is multiple sclerosis, rheumatoid arthritis, psoriasis, juvenile rheumatoid arthritis, osteoarthritis, psoriatic arthritis, ankylosing spondylitis, systemic lupus erythematosus, Hashimoto's disease, Graves disease, inflammatory bowel disease, pancreatitis, Crohn's disease, autoimmune diabetes, autoimmune ocular disease, ulcerative colitis, irritable bowel syndrome (IBS), inflammatory bowel disease (IBD), uveitis, or scleritis.

In some embodiments of these methods, the SGK1 inhibitor is a small molecule, a blocking antibody or antigen-binding fragment thereof, a polypeptide, an antisense oligonucleotide, an RNA molecule, an aptamer, or a ribozyme.

In some embodiments, the small molecule is a small molecule of Formula (I):

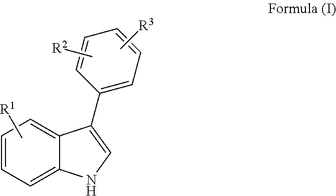

wherein R1 is optionally substituted phenyl, optionally substituted β-napthyl, or optionally substituted 3-CN-phenyl;
wherein R2 is CO₂R4 or C(R4,R5) CO₂R4;
wherein R3 and R4 are independently absent, H, C₁-C₆ alkyl, or C₅-C₈ cycloalkyl; each of which may be optionally substituted;
wherein R5 and R6 are independently absent, H, or C₁-C₆ alkyl, each of which may be optionally substituted; and
pharmaceutically acceptable salts thereof.

In some embodiments, the small molecule is a small molecule of Formula (Ia):

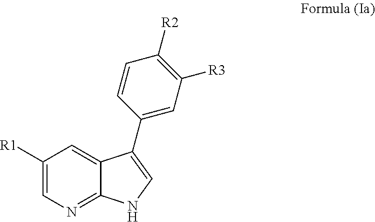

In some embodiments, R1 is phenyl, R2 is CO₂H, and R3 is H. In some embodiments, R1 is phenyl, R2 is CO₂H, and R3 is

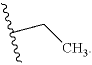 [SMILES: *CC]

In some embodiments, R1 is phenyl, R2 is CO₂H, and R3 is

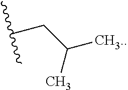 [SMILES: *CC(C)C]

In some embodiments, R1 is phenyl, R2 is CO₂H, and R3 is

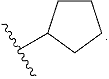 [SMILES: *C1CCCC1]

In some embodiments, R1 is β-napthyl, R2 is CH₂CO₂H, and R3 is H. In some embodiments, R1 is β-napthyl, R2 is

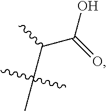 [SMILES: *C(C)C(=O)O]

and R3 is H. In some embodiments, R1 is β-napthyl, R2 is

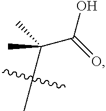

and R3 is H. In some embodiments, R1 is phenyl, R2 is

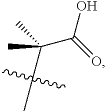

and R3 is H. In some embodiments, R1 is 3-CN-phenyl, R2 is

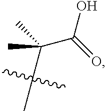

### and R3 is H.

In some embodiments of these methods, the small molecule is selected from the group consisting of 3-(4-hydroxy-3-methylphenylamino)-4-[(R)-1-(3-hydroxyphenyl)ethylamino]cyclobut-3-ene-1,2-dione; 3-(3-amino-1-tert-butyloxycarbonylindazol-5-ylamino)-4-(3-hydroxybenzylamino)cyclobut-3-ene-1,2-dione; 3-(3-amino-1-tert-butyloxycarbonylindazol-5-ylamino)-4-(3-methoxybenzylamino)cyclobut-3-ene-1,2-dione; 3-(3-amino-1H-indazol-5-ylamino)-4-(3-methoxybenzylamino)cyclobut-3-ene-1,2-dione; 3-(1H-indazol-5-ylamino)-4-[(R)-1-(3-methoxyphenyl)ethylamino]cyclo-but-3-ene-1,2-dione; 3-(1H-indazol-5-ylamino)-4-(3-methoxybenzylamino)cyclobut-3-ene-1,2-dione; 3-(1H-indazol-5-ylamino)-4-(3-hydroxybenzylamino)cyclobut-3-ene-1,2-dione; 3-(1-ethylaminocarbonylindazol-5-ylamino)-4-(3-methoxybenzylamino)cyclobut-3-ene-1,2-dione; 3-(1-ethylaminocarbonylindazol-5-ylamino)-4-(3-hydroxybenzylamino)cyclobut-3-ene-1,2-dione; 3-(3-amino-1H-indazol-5-ylamino)-4-[(R)-1-(3-methoxyphenyl)ethylamino]cyclobut-3-ene-1,2-dione; 3-(3-amino-1H-indazol-5-ylamino)-4-[(R)-1-(3-hydroxyphenyl)ethylamino]cyclobut-3-ene-1,2-dione; 3-(3-amino-1H-indazol-5-ylamino)-4-[(R)-1-(3-chlorophenyl)ethylamino]cyclobut-3-ene-1,2-dione; 3-(3-amino-1H-indazol-5-ylamino)-4-(3-chlorobenzylamino)cyclobut-3-ene-1,2-dione; 3-(3-amino-1H-indazol-5-ylamino)-4-(3-trifluoromethylbenzylamino)cyclobut-3-ene-1,2-dione; 3-(3-amino-1H-indazol-5-ylamino)-4-(3-trifluoromethoxybenzylamino)-cyclobut-3-ene-1,2-dione; 3-(3-amino-1H-indazol-5-ylamino)-4-(3-aminosulfonylbenzylamino)cyclobut-3-ene-1,2-dione; 3-(3-amino-1H-indazol-5-ylamino)-4-[(2-hydroxypyridin-4-ylmethyl)amino]cyclobut-3-ene-1,2-dione; 3-(3-amino-7-methyl-1H-indazol-5-ylamino)-4-[(R)-1-(3-methoxyphenyl)ethylamino]cyclobut-3-ene-1,2-dione; 3-(3-amino-7-methyl-1H-indazol-5-ylamino)-4-[(R)-1-(3-hydroxyphenyl)ethylamino]cyclobut-3-ene-1,2-dione; 3-(3-amino-7-methyl-1H-indazol-5-ylamino)-4-(3-aminosulfonylbenzylamino)cyclobut-3-ene-1,2-dione; 3-(3-amino-7-methyl-1H-indazol-5-ylamino)-4-[(2-hydroxypyridin-4-ylmethyl)amino]cyclobut-3-ene-1,2-dione; 3-(3-amino-7-methyl-1H-indazol-5-ylamino)-4-(3-methoxybenzylamino)-cyclobut-3-ene-1,2-dione; 3-(3-amino-7-methyl-1H-indazol-5-ylamino)-4-(3-hydroxybenzylamino)-cyclobut-3-ene-1,2-dione; 3-[3-(morpholin-4-yl)-1H-indazol-5-ylamino]-4-[(R)-1-(3-hydroxyphenyl)ethylamino]cyclobut-3-ene-1,2-dione; 3-[3-(piperidin-1-yl)-1H-indazol-5-ylamino]-4-[(R)-1-(3-hydroxyphenyl)ethylamino]cyclobut-3-ene-1,2-dione; 3-[3-(pyrrolidin-1-yl)-1H-indazol-5-ylamino]-4-[(R)-1-(3-hydroxyphenyl)ethylamino]cyclobut-3-ene-1,2-dione; 3-(3-bromo-1H-indazol-5-ylamino)-4-[(R)-1-(3-hydroxyphenyl)ethylamino]cyclobut-3-ene-1,2-dione; 3-(3-acetamido-1H-indazol-5-ylamino)-4-[(R)-1-(3-hydroxyphenyl)ethylamino]cyclobut-3-ene-1,2-dione; 3-(1H-indazol-5-ylamino)-4-[(R)-1-(3-hydroxyphenyl)ethylamino]cyclobut-3-ene-1,2-dione; 3-(7-bromo-1H-indazol-5-ylamino)-4-(3-methoxybenzylamino)cyclobut-3-ene-1,2-dione; 3-(7-bromo-1H-indazol-5-ylamino)-4-(3-hydroxybenzylamino)cyclobut-3-ene-1,2-dione; 3-(1H-indazol-5-ylamino)-4-(3-chlorobenzylamino)cyclobut-3-ene-1,2-dione; 3-(7-methyl-1H-indazol-5-ylamino)-4-(3-hydroxybenzylamino)cyclobut-3-ene-1,2-dione; 3-(7-methyl-1H-indazol-5-ylamino)-4-[(R)-1-(3-methoxyphenyl)ethylamino]cyclobut-3-ene-1,2-dione; 3-(7-methyl-1H-indazol-5-ylamino)-4-[(S)-1-(3-methoxyphenyl)ethylamino]cyclobut-3-ene-1,2-dione; 3-(7-methyl-1H-indazol-5-ylamino)-4-[(R)-1-(3-hydroxyphenyl)ethylamino]cyclobut-3-ene-1,2-dione; 3-(7-methyl-1H-indazol-5-ylamino)-4-(3-methoxybenzylamino)cyclobut-3-ene-1,2-dione; 3-(1H-indazol-5-ylamino)-4-(3-aminosulfonylbenzylamino)cyclobut-3-ene-1,2-dione; 3-(1H-indazol-5-ylamino)-4-[(R)-1-(3-hydroxyphenyl)-ethylamino]cyclobut-3-ene-1,2-dione; 3-(3-benzoylamino-1H-indazol-5-ylamino)-4-(3-hydroxybenzylamino)cyclobut-3-ene-1,2-dione; 3-(3-benzoylamino-1H-indazol-5-ylamino)-4-(3-methoxybenzylamino)cyclobut-3-ene-1,2-dione; 3-(3-benzoylamino-1H-indazol-5-ylamino)-4-[(R)-1-(3-methoxyphenyl)ethylamino]cyclobut-3-ene-1,2-dione; 3-(3-benzoylamino-1H-indazol-5-ylamino)-4-[(R)-1-(3-hydroxyphenyl)-ethylamino]cyclobut-3-ene-1,2-dione; 3-[3-(3-chlorobenzoylamino)-1H-indazol-5-ylamino]-4-[(R)-1-(3-meth-oxyphenyl)ethylamino]cyclobut-3-ene-1,2-dione; 3-[3-(3-chlorobenzoylamino)-1H-indazol-5-ylamino]-4-[(R)-1-(3-hydroxyphenyl)ethylamino]cyclobut-3-ene-1,2-dione; 3-[3-(3-chlorobenzoylamino)-1H-indazol-5-ylamino]-4-[(R)-1-(3-fluorophenyl)ethylamino]cyclobut-3-ene-1,2-dione; 3-[3-(3-chlorobenzoylamino)-1H-indazol-5-ylamino]-4-[(R)-1-(3-acet-amidophenyl)ethylamino]cyclobut-3-ene-1,2-dione; 3-[3-(3-chlorobenzoylamino)-1H-indazol-5-ylamino]-4-(3-methoxybenzylamino)cyclobut-3-ene-1,2-dione; 3-[3-(3-chlorobenzoylamino)-1H-indazol-5-ylamino]-4-(3-hydroxybenzylamino)cyclobut-3-ene-1,2-dione; 3-[3-(3-chlorobenzoylamino)-1H-indazol-5-ylamino]-4-(3-fluorobenzylamino)cyclobut-3-ene-1,2-dione; 3-[3-(3-chlorobenzoylamino)-1H-indazol-5-ylamino]-4-(3-acetamidobenzylamino)cyclobut-3-ene-1,2-dione; 3-(3-benzoylamino-1H-indazol-5-ylamino)-4-[(R)-1-(2,3-difluorophen-yl)ethylamino]cyclobut-3-ene-1,2-dione; 3-(3-benzoylamino-1H-indazol-5-ylamino)-4-[(R)-1-(3-methylsulfonamidophenyl)ethylamino]cyclobut-3-ene-1,2-dione; 3-[3-(3-chlorobenzoylamino)-1H-indazol-5-ylamino]-4-[(R)-1-(2,3-difluorophenyl)ethylamino]cyclobut-3-ene-1,2-dione; 3-[3-(3-chlorobenzoylamino)-1H-indazol-5-ylamino]-4-[(R)-1-(3-methylsulfonamidophenyl)ethylamino]cyclobut-3-ene-1,2-dione; 3-(3-benzoylamino-1H-indazol-5-ylamino)-4-(2,3-difluorobenzylamino-)cyclobut-3-ene-1,2-dione; 3-(3-benzoylamino-1H-indazol-5-ylamino)-4-(3-methylsulfonamidobenzylamino)cyclobut-3-ene-1,2-dione; 3-[3-(3-chlorobenzoylamino)-1H-indazol-5-ylamino]-4-(2,3-difluorob-enzylamino)cyclobut-3-ene-1,2-dione; and 3-[3-(3-chlorobenzoylamino)-1H-indazol-5-ylamino]-4-(3-methylsulfonamidobenzylamino)cyclobut-3-ene-1,2-dione.

In some embodiments of these methods, the method of inhibiting a TH17-mediated immune response further comprising administering to the subject in need thereof a therapeutic agent selected from the group consisting of a cytokine inhibitor, a growth factor inhibitor, a chemotherapeutic agent, an immunosuppressant, an anti-inflammatory agent, a metabolic inhibitor, an enzyme inhibitor, a cytotoxic agent, and a cytostatic agent.

In one aspect, described herein are uses of an SGK1 inhibitor in inhibiting a TH17 cell-mediated immune response in a subject in need thereof.

In some embodiments of the uses of SGK1 inhibitors, the TH17 cell-mediated response being inhibited comprises expression or production of IL-17 by a TH17 cell. In some embodiments, the TH17 cell-mediated response being inhibited further comprises expression or production of one or more of IL-17F, IL-22, IL-26, IL-21, and TNF-α.

In some embodiments of the uses of SGK1 inhibitors, the TH17 cell-mediated response being inhibited comprises inhibition of proliferation of or expansion of a TH17 cell. In some embodiments of the uses of SGK1 inhibitors, the TH17 cell-mediated response being inhibited comprises trafficking of a TH17 cell.

In some embodiments of the uses of SGK1 inhibitors, the subject in need of inhibition of a TH17-mediated immune response has a TH17-mediated disorder. In some such embodiments, the TH17-mediated disorder is an autoimmune disease or a chronic inflammatory disease. In some embodiments, the autoimmune disease is multiple sclerosis, rheumatoid arthritis, psoriasis, juvenile rheumatoid arthritis, osteoarthritis, psoriatic arthritis, ankylosing spondylitis, systemic lupus erythematosus, Hashimoto's disease, Graves disease, inflammatory bowel disease, pancreatitis, Crohn's disease, autoimmune diabetes, autoimmune ocular disease, ulcerative colitis, irritable bowel syndrome (IBS), inflammatory bowel disease (IBD), uveitis, or scleritis.

In some embodiments of the uses of SGK1 inhibitors, the SGK1 inhibitor is a small molecule, a blocking antibody or antigen-binding fragment thereof, a polypeptide, an antisense oligonucleotide, an RNA molecule, an aptamer, or a ribozyme.

In some such embodiments, the small molecule is a small molecule of Formula (I):

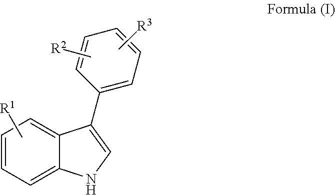

wherein R1 is optionally substituted phenyl, optionally substituted β-napthyl, or optionally substituted 3-CN-phenyl;
wherein R2 is CO₂R4 or C(R4,R5) CO₂R4;
wherein R3 and R4 are independently absent, H, C₁-C₆ alkyl, or C₅-C₈ cycloalkyl; each of which may be optionally substituted;
wherein R5 and R6 are independently absent, H, or C₁-C₆ alkyl, each of which may be optionally substituted; and
pharmaceutically acceptable salts thereof.

In some embodiments, the small molecule is a small molecule of Formula (Ia):

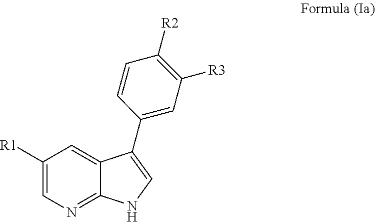

In some embodiments of the small molecule of Formula (Ia), R1 is phenyl, R2 is CO₂H, and R3 is H. In some embodiments, R1 is phenyl, R2 is CO₂, and R3 is

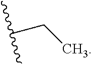 [SMILES: *CC]

In some embodiments, R1 is phenyl, R2 is CO₂H, and R3 is

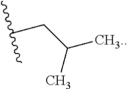 [SMILES: *CC(C)C]

In some embodiments, R1 is phenyl, R2 is CO₂H, and R3 is

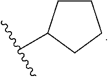 [SMILES: *C1CCCC1]

In some embodiments, R1 is β-napthyl, R2 is CH₂CO₂H, and R3 is H. In some embodiments, R1 is β-napthyl, R2 is

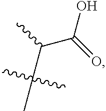 [SMILES: *C(C)C(=O)O]

and R3 is H. In some embodiments, R1 is β-napthyl, R2 is

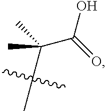

and R3 is H. In some embodiments, R1 is phenyl, R2 is

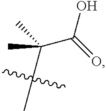

and R3 is H. In some embodiments, R1 is 3-CN-phenyl, R2 is

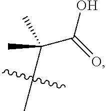

### and R3 is H.

In some embodiments of the uses of the SGK1 inhibitors, the small molecule is selected from the group consisting of 3-(4-hydroxy-3-methylphenylamino)-4-[(R)-1-(3-hydroxyphenyl)ethylamino]cyclobut-3-ene-1,2-dione; 3-(3-amino-1-tert-butyloxycarbonylindazol-5-ylamino)-4-(3-hydroxybenzylamino)cyclobut-3-ene-1,2-dione; 3-(3-amino-1-tert-butyloxycarbonylindazol-5-ylamino)-4-(3-methoxybenzylamino)cyclobut-3-ene-1,2-dione; 3-(3-amino-1H-indazol-5-ylamino)-4-(3-methoxybenzylamino)cyclobut-3-ene-1,2-dione; 3-(1H-indazol-5-ylamino)-4-[(R)-1-(3-methoxyphenyl)ethylamino]cyclo-but-3-ene-1,2-dione; 3-(1H-indazol-5-ylamino)-4-(3-methoxybenzylamino)cyclobut-3-ene-1,2-dione; 3-(1H-indazol-5-ylamino)-4-(3-hydroxybenzylamino)cyclobut-3-ene-1,2-dione; 3-(1-ethylaminocarbonylindazol-5-ylamino)-4-(3-methoxybenzylamino)cyclobut-3-ene-1,2-dione; 3-(1-ethylaminocarbonylindazol-5-ylamino)-4-(3-hydroxybenzylamino)cyclobut-3-ene-1,2-dione; 3-(3-amino-1H-indazol-5-ylamino)-4-[(R)-1-(3-methoxyphenyl)ethylamino]cyclobut-3-ene-1,2-dione; 3-(3-amino-1H-indazol-5-ylamino)-4-[(R)-1-(3-hydroxyphenyl)ethylamino]cyclobut-3-ene-1,2-dione; 3-(3-amino-1H-indazol-5-ylamino)-4-[(R)-1-(3-chlorophenyl)ethylamino]cyclobut-3-ene-1,2-dione; 3-(3-amino-1H-indazol-5-ylamino)-4-(3-chlorobenzylamino)cyclobut-3-ene-1,2-dione; 3-(3-amino-1H-indazol-5-ylamino)-4-(3-trifluoromethylbenzylamino)cyclobut-3-ene-1,2-dione; 3-(3-amino-1H-indazol-5-ylamino)-4-(3-trifluoromethoxybenzylamino)-cyclobut-3-ene-1,2-dione; 3-(3-amino-1H-indazol-5-ylamino)-4-(3-aminosulfonylbenzylamino)cyclobut-3-ene-1,2-dione; 3-(3-amino-1H-indazol-5-ylamino)-4-[(2-hydroxypyridin-4-ylmethyl)amino]cyclobut-3-ene-1,2-dione; 3-(3-amino-7-methyl-1H-indazol-5-ylamino)-4-[(R)-1-(3-methoxyphenyl)ethylamino]cyclobut-3-ene-1,2-dione; 3-(3-amino-7-methyl-1H-indazol-5-ylamino)-4-[(R)-1-(3-hydroxyphenyl)ethylamino]cyclobut-3-ene-1,2-dione; 3-(3-amino-7-methyl-1H-indazol-5-ylamino)-4-(3-aminosulfonylbenzylamino)cyclobut-3-ene-1,2-dione; 3-(3-amino-7-methyl-1H-indazol-5-ylamino)-4-[(2-hydroxypyridin-4-ylmethyl)amino]cyclobut-3-ene-1,2-dione; 3-(3-amino-7-methyl-1H-indazol-5-ylamino)-4-(3-methoxybenzylamino)-cyclobut-3-ene-1,2-dione; 3-(3-amino-7-methyl-1H-indazol-5-ylamino)-4-(3-hydroxybenzylamino)-cyclobut-3-ene-1,2-dione; 3-[3-(morpholin-4-yl)-1H-indazol-5-ylamino]-4-[(R)-1-(3-hydroxyphenyl)ethylamino]cyclobut-3-ene-1,2-dione; 3-[3-(piperidin-1-yl)-1H-indazol-5-ylamino]-4-[(R)-1-(3-hydroxyphenyl)ethylamino]cyclobut-3-ene-1,2-dione; 3-[3-(pyrrolidin-1-yl)-1H-indazol-5-ylamino]-4-[(R)-1-(3-hydroxyphenyl)ethylamino]cyclobut-3-ene-1,2-dione; 3-(3-bromo-1H-indazol-5-ylamino)-4-[(R)-1-(3-hydroxyphenyl)ethylamino]cyclobut-3-ene-1,2-dione; 3-(3-acetamido-1H-indazol-5-ylamino)-4-[(R)-1-(3-hydroxyphenyl)ethylamino]cyclobut-3-ene-1,2-dione; 3-(1H-indazol-5-ylamino)-4-[(R)-1-(3-hydroxyphenyl)ethylamino]cyclobut-3-ene-1,2-dione; 3-(7-bromo-1H-indazol-5-ylamino)-4-(3-methoxybenzylamino)cyclobut-3-ene-1,2-dione; 3-(7-bromo-1H-indazol-5-ylamino)-4-(3-hydroxybenzylamino)cyclobut-3-ene-1,2-dione; 3-(1H-indazol-5-ylamino)-4-(3-chlorobenzylamino)cyclobut-3-ene-1,2-dione; 3-(7-methyl-1H-indazol-5-ylamino)-4-(3-hydroxybenzylamino)cyclobut-3-ene-1,2-dione; 3-(7-methyl-1H-indazol-5-ylamino)-4-[(R)-1-(3-methoxyphenyl)ethylamino]cyclobut-3-ene-1,2-dione; 3-(7-methyl-1H-indazol-5-ylamino)-4-[(S)-1-(3-methoxyphenyl)ethylamino]cyclobut-3-ene-1,2-dione; 3-(7-methyl-1H-indazol-5-ylamino)-4-[(R)-1-(3-hydroxyphenyl)ethylamino]cyclobut-3-ene-1,2-dione; 3-(7-methyl-1H-indazol-5-ylamino)-4-(3-methoxybenzylamino)cyclobut-3-ene-1,2-dione; 3-(1H-indazol-5-ylamino)-4-(3-aminosulfonylbenzylamino)cyclobut-3-ene-1,2-dione; 3-(1H-indazol-5-ylamino)-4-[(R)-1-(3-hydroxyphenyl)-ethylamino]cyclobut-3-ene-1,2-dione; 3-(3-benzoylamino-1H-indazol-5-ylamino)-4-(3-hydroxybenzylamino)cyclobut-3-ene-1,2-dione; 3-(3-benzoylamino-1H-indazol-5-ylamino)-4-(3-methoxybenzylamino)cyclobut-3-ene-1,2-dione; 3-(3-benzoylamino-1H-indazol-5-ylamino)-4-[(R)-1-(3-methoxyphenyl)ethylamino]cyclobut-3-ene-1,2-dione; 3-(3-benzoylamino-1H-indazol-5-ylamino)-4-[(R)-1-(3-hydroxyphenyl)-ethylamino]cyclobut-3-ene-1,2-dione; 3-[3-(3-chlorobenzoylamino)-1H-indazol-5-ylamino]-4-[(R)-1-(3-meth-oxyphenyl)ethylamino]cyclobut-3-ene-1,2-dione; 3-[3-(3-chlorobenzoylamino)-1H-indazol-5-ylamino]-4-[(R)-1-(3-hydroxyphenyl)ethylamino]cyclobut-3-ene-1,2-dione; 3-[3-(3-chlorobenzoylamino)-1H-indazol-5-ylamino]-4-[(R)-1-(3-fluorophenyl)ethylamino]cyclobut-3-ene-1,2-dione; 3-[3-(3-chlorobenzoylamino)-1H-indazol-5-ylamino]-4-[(R)-1-(3-acet-amidophenyl)ethylamino]cyclobut-3-ene-1,2-dione; 3-[3-(3-chlorobenzoylamino)-1H-indazol-5-ylamino]-4-(3-methoxybenzylamino)cyclobut-3-ene-1,2-dione; 3-[3-(3-chlorobenzoylamino)-1H-indazol-5-ylamino]-4-(3-hydroxybenzylamino)cyclobut-3-ene-1,2-dione; 3-[3-(3-chlorobenzoylamino)-1H-indazol-5-ylamino]-4-(3-fluorobenzylamino)cyclobut-3-ene-1,2-dione; 3-[3-(3-chlorobenzoylamino)-1H-indazol-5-ylamino]-4-(3-acetamidobenzylamino)cyclobut-3-ene-1,2-dione; 3-(3-benzoylamino-1H-indazol-5-ylamino)-4-[(R)-1-(2,3-difluorophen-yl)ethylamino]cyclobut-3-ene-1,2-dione; 3-(3-benzoylamino-1H-indazol-5-ylamino)-4-[(R)-1-(3-methylsulfonamidophenyl)ethylamino]cyclobut-3-ene-1,2-dione; 3-[3-(3-chlorobenzoylamino)-1H-indazol-5-ylamino]-4-[(R)-1-(2,3-difluorophenyl)ethylamino]cyclobut-3-ene-1,2-dione; 3-[3-(3-chlorobenzoylamino)-1H-indazol-5-ylamino]-4-[(R)-1-(3-methylsulfonamidophenyl)ethylamino]cyclobut-3-ene-1,2-dione; 3-(3-benzoylamino-1H-indazol-5-ylamino)-4-(2,3-difluorobenzylamino-)cyclobut-3-ene-1,2-dione; 3-(3-benzoylamino-1H-indazol-5-ylamino)-4-(3-methylsulfonamidobenzylamino)cyclobut-3-ene-1,2-dione; 3-[3-(3-chlorobenzoylamino)-1H-indazol-5-ylamino]-4-(2,3-difluorob-enzylamino)cyclobut-3-ene-1,2-dione; and 3-[3-(3-chlorobenzoylamino)-1H-indazol-5-ylamino]-4-(3-methylsulfonamidobenzylamino)cyclobut-3-ene-1,2-dione.

In another aspect, described herein are methods of modulating a TH17 cell-mediated immune response in a subject in need thereof, such methods comprising administering to a subject in need thereof a therapeutically effective amount of an agent that modulates serum and glucocorticoid-regulated kinase 1 (SGK1) activity to modulate a TH17 cell-mediated immune response.

In another aspect, described herein are methods of modulating TH17 cell-mediated cytokine production in a subject in need thereof, such methods comprising administering to a subject in need thereof a therapeutically effective amount of an agent that modulates serum and glucocorticoid-regulated kinase 1 (SGK1) activity to modulate TH17 cell-mediated cytokine production.

In another aspect, described herein are methods of modulating TH17 cell proliferation in a subject in need thereof, such methods comprising administering to a subject in need thereof a therapeutically effective amount of an agent that modulates serum and glucocorticoid-regulated kinase 1 (SGK1) activity to modulate TH17 cell proliferation.

In another aspect, described herein are methods of modulating TH17 cell-mediated inflammatory activity in a subject in need thereof, such methods comprising administering to a subject in need thereof a therapeutically effective amount of an agent that modulates serum and glucocorticoid-regulated kinase 1 (SGK1) activity to modulate TH17 cell-mediated inflammatory activity.

In another aspect, described herein are methods of modulating TH17 cell migration in a subject in need thereof, such methods comprising administering to a subject in need thereof a therapeutically effective amount of an agent that modulates serum and glucocorticoid-regulated kinase 1 (SGK1) activity to modulate TH17 cell migration.

In some embodiments of any of these methods, the method further comprises administering to the subject in need another therapeutic agent selected from the group consisting of a cytokine inhibitor, a growth factor inhibitor, an immunosuppressant, an anti-inflammatory agent, a metabolic inhibitor, an enzyme inhibitor, a cytotoxic agent, and a cytostatic agent.

In some embodiments of these methods, the agent is an inhibitor of SGK1 that decreases SGK1 activity. In some such embodiments of these methods, the inhibitor of SGK1 is a an antibody or antigen-binding fragment thereof, a polypeptide, a small molecule, an antisense oligonucleotide, an RNA molecule, an aptamer, or a ribozyme.

In some embodiments, the small molecule is a small molecule of Formula (I):

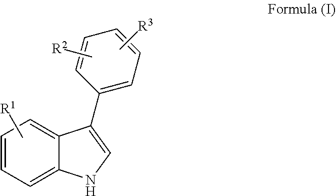

wherein R1 is optionally substituted phenyl, optionally substituted β-napthyl, or optionally substituted 3-CN-phenyl;
wherein R2 is CO₂R4 or C(R4,R5) CO₂R4;
wherein R3 and R4 are independently H, C₁-C₆ alkyl, or C₅-C₈ cycloalkyl;
and wherein R5 and R6 are independently H or C₁-C₆ alkyl.

In some embodiments, the small molecule is a small molecule of Formula (Ia):

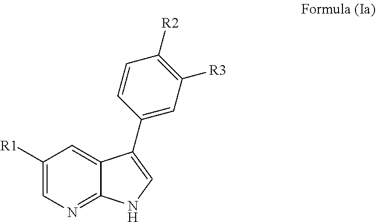

In some embodiments of the small molecule of Formula (Ia), R1 is phenyl, R2 is CO₂H, and R3 is H. In some embodiments, R1 is phenyl, R2 is CO₂, and R3 is

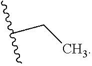 [SMILES: *CC]

In some embodiments, R1 is phenyl, R2 is CO₂H, and R3 is

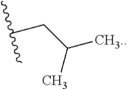 [SMILES: *CC(C)C]

In some embodiments, R1 is phenyl, R2 is CO₂H, and R3 is

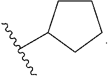 [SMILES: *C1CCCC1]

In some embodiments, R1 is β-napthyl, R2 is CH₂CO₂H, and R3 is H. In some embodiments, R1 is β-napthyl, R2 is

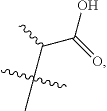 [SMILES: *C(C)C(=O)O]

and R3 is H. In some embodiments, R1 is β-napthyl, R2 is

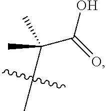

and R3 is H. In some embodiments, R1 is phenyl, R2 is

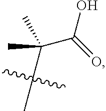

and R3 is H. In some embodiments, R1 is 3-CN-phenyl, R2 is

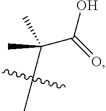

### and R3 is H.

In some embodiments of these methods, the small molecule is selected from the group consisting of 3-(4-hydroxy-3-methylphenylamino)-4-[(R)-1-(3-hydroxyphenyl)ethylamino]cyclobut-3-ene-1,2-dione; 3-(3-amino-1-tert-butyloxycarbonylindazol-5-ylamino)-4-(3-hydroxybenzylamino)cyclobut-3-ene-1,2-dione; 3-(3-amino-1-tert-butyloxycarbonylindazol-5-ylamino)-4-(3-methoxybenzylamino)cyclobut-3-ene-1,2-dione; 3-(3-amino-1H-indazol-5-ylamino)-4-(3-methoxybenzylamino)cyclobut-3-ene-1,2-dione; 3-(1H-indazol-5-ylamino)-4-[(R)-1-(3-methoxyphenyl)ethylamino]cyclo-but-3-ene-1,2-dione; 3-(1H-indazol-5-ylamino)-4-(3-methoxybenzylamino)cyclobut-3-ene-1,2-dione; 3-(1H-indazol-5-ylamino)-4-(3-hydroxybenzylamino)cyclobut-3-ene-1,2-dione; 3-(1-ethylaminocarbonylindazol-5-ylamino)-4-(3-methoxybenzylamino)cyclobut-3-ene-1,2-dione; 3-(1-ethylaminocarbonylindazol-5-ylamino)-4-(3-hydroxybenzylamino)cyclobut-3-ene-1,2-dione; 3-(3-amino-1H-indazol-5-ylamino)-4-[(R)-1-(3-methoxyphenyl)ethylamino]cyclobut-3-ene-1,2-dione; 3-(3-amino-1H-indazol-5-ylamino)-4-[(R)-1-(3-hydroxyphenyl)ethylamino]cyclobut-3-ene-1,2-dione; 3-(3-amino-1H-indazol-5-ylamino)-4-[(R)-1-(3-chlorophenyl)ethylamino]cyclobut-3-ene-1,2-dione; 3-(3-amino-1H-indazol-5-ylamino)-4-(3-chlorobenzylamino)cyclobut-3-ene-1,2-dione; 3-(3-amino-1H-indazol-5-ylamino)-4-(3-trifluoromethylbenzylamino)cyclobut-3-ene-1,2-dione; 3-(3-amino-1H-indazol-5-ylamino)-4-(3-trifluoromethoxybenzylamino)-cyclobut-3-ene-1,2-dione; 3-(3-amino-1H-indazol-5-ylamino)-4-(3-aminosulfonylbenzylamino)cyclobut-3-ene-1,2-dione; 3-(3-amino-1H-indazol-5-ylamino)-4-[(2-hydroxypyridin-4-ylmethyl)amino]cyclobut-3-ene-1,2-dione; 3-(3-amino-7-methyl-1H-indazol-5-ylamino)-4-[(R)-1-(3-methoxyphenyl)ethylamino]cyclobut-3-ene-1,2-dione; 3-(3-amino-7-methyl-1H-indazol-5-ylamino)-4-[(R)-1-(3-hydroxyphenyl)ethylamino]cyclobut-3-ene-1,2-dione; 3-(3-amino-7-methyl-1H-indazol-5-ylamino)-4-(3-aminosulfonylbenzylamino)cyclobut-3-ene-1,2-dione; 3-(3-amino-7-methyl-1H-indazol-5-ylamino)-4-[(2-hydroxypyridin-4-ylmethyl)amino]cyclobut-3-ene-1,2-dione; 3-(3-amino-7-methyl-1H-indazol-5-ylamino)-4-(3-methoxybenzylamino)-cyclobut-3-ene-1,2-dione; 3-(3-amino-7-methyl-1H-indazol-5-ylamino)-4-(3-hydroxybenzylamino)-cyclobut-3-ene-1,2-dione; 3-[3-(morpholin-4-yl)-1H-indazol-5-ylamino]-4-[(R)-1-(3-hydroxyphenyl)ethylamino]cyclobut-3-ene-1,2-dione; 3-[3-(piperidin-1-yl)-1H-indazol-5-ylamino]-4-[(R)-1-(3-hydroxyphenyl)ethylamino]cyclobut-3-ene-1,2-dione; 3-[3-(pyrrolidin-1-yl)-1H-indazol-5-ylamino]-4-[(R)-1-(3-hydroxyphenyl)ethylamino]cyclobut-3-ene-1,2-dione; 3-(3-bromo-1H-indazol-5-ylamino)-4-[(R)-1-(3-hydroxyphenyl)ethylamino]cyclobut-3-ene-1,2-dione; 3-(3-acetamido-1H-indazol-5-ylamino)-4-[(R)-1-(3-hydroxyphenyl)ethylamino]cyclobut-3-ene-1,2-dione; 3-(1H-indazol-5-ylamino)-4-[(R)-1-(3-hydroxyphenyl)ethylamino]cyclobut-3-ene-1,2-dione; 3-(7-bromo-1H-indazol-5-ylamino)-4-(3-methoxybenzylamino)cyclobut-3-ene-1,2-dione; 3-(7-bromo-1H-indazol-5-ylamino)-4-(3-hydroxybenzylamino)cyclobut-3-ene-1,2-dione; 3-(1H-indazol-5-ylamino)-4-(3-chlorobenzylamino)cyclobut-3-ene-1,2-dione; 3-(7-methyl-1H-indazol-5-ylamino)-4-(3-hydroxybenzylamino)cyclobut-3-ene-1,2-dione; 3-(7-methyl-1H-indazol-5-ylamino)-4-[(R)-1-(3-methoxyphenyl)ethylamino]cyclobut-3-ene-1,2-dione; 3-(7-methyl-1H-indazol-5-ylamino)-4-[(S)-1-(3-methoxyphenyl)ethylamino]cyclobut-3-ene-1,2-dione; 3-(7-methyl-1H-indazol-5-ylamino)-4-[(R)-1-(3-hydroxyphenyl)ethylamino]cyclobut-3-ene-1,2-dione; 3-(7-methyl-1H-indazol-5-ylamino)-4-(3-methoxybenzylamino)cyclobut-3-ene-1,2-dione; 3-(1H-indazol-5-ylamino)-4-(3-aminosulfonylbenzylamino)cyclobut-3-ene-1,2-dione; 3-(1H-indazol-5-ylamino)-4-[(R)-1-(3-hydroxyphenyl)-ethylamino]cyclobut-3-ene-1,2-dione; 3-(3-benzoylamino-1H-indazol-5-ylamino)-4-(3-hydroxybenzylamino)cyclobut-3-ene-1,2-dione; 3-(3-benzoylamino-1H-indazol-5-ylamino)-4-(3-methoxybenzylamino)cyclobut-3-ene-1,2-dione; 3-(3-benzoylamino-1H-indazol-5-ylamino)-4-[(R)-1-(3-methoxyphenyl)ethylamino]cyclobut-3-ene-1,2-dione; 3-(3-benzoylamino-1H-indazol-5-ylamino)-4-[(R)-1-(3-hydroxyphenyl)-ethylamino]cyclobut-3-ene-1,2-dione; 3-[3-(3-chlorobenzoylamino)-1H-indazol-5-ylamino]-4-[(R)-1-(3-meth-oxyphenyl)ethylamino]cyclobut-3-ene-1,2-dione; 3-[3-(3-chlorobenzoylamino)-1H-indazol-5-ylamino]-4-[(R)-1-(3-hydroxyphenyl)ethylamino]cyclobut-3-ene-1,2-dione; 3-[3-(3-chlorobenzoylamino)-1H-indazol-5-ylamino]-4-[(R)-1-(3-fluorophenyl)ethylamino]cyclobut-3-ene-1,2-dione; 3-[3-(3-chlorobenzoylamino)-1H-indazol-5-ylamino]-4-[(R)-1-(3-acet-amidophenyl)ethylamino]cyclobut-3-ene-1,2-dione; 3-[3-(3-chlorobenzoylamino)-1H-indazol-5-ylamino]-4-(3-methoxybenzylamino)cyclobut-3-ene-1,2-dione; 3-[3-(3-chlorobenzoylamino)-1H-indazol-5-ylamino]-4-(3-hydroxybenzylamino)cyclobut-3-ene-1,2-dione; 3-[3-(3-chlorobenzoylamino)-1H-indazol-5-ylamino]-4-(3-fluorobenzylamino)cyclobut-3-ene-1,2-dione; 3-[3-(3-chlorobenzoylamino)-1H-indazol-5-ylamino]-4-(3-acetamidobenzylamino)cyclobut-3-ene-1,2-dione; 3-(3-benzoylamino-1H-indazol-5-ylamino)-4-[(R)-1-(2,3-difluorophen-yl)ethylamino]cyclobut-3-ene-1,2-dione; 3-(3-benzoylamino-1H-indazol-5-ylamino)-4-[(R)-1-(3-methylsulfonamidophenyl)ethylamino]cyclobut-3-ene-1,2-dione; 3-[3-(3-chlorobenzoylamino)-1H-indazol-5-ylamino]-4-[(R)-1-(2,3-difluorophenyl)ethylamino]cyclobut-3-ene-1,2-dione; 3-[3-(3-chlorobenzoylamino)-1H-indazol-5-ylamino]-4-[(R)-1-(3-methylsulfonamidophenyl)ethylamino]cyclobut-3-ene-1,2-dione; 3-(3-benzoylamino-1H-indazol-5-ylamino)-4-(2,3-difluorobenzylamino-)cyclobut-3-ene-1,2-dione; 3-(3-benzoylamino-1H-indazol-5-ylamino)-4-(3-methylsulfonamidobenzylamino)cyclobut-3-ene-1,2-dione; 3-[3-(3-chlorobenzoylamino)-1H-indazol-5-ylamino]-4-(2,3-difluorob-enzylamino)cyclobut-3-ene-1,2-dione; and 3-[3-(3-chlorobenzoylamino)-1H-indazol-5-ylamino]-4-(3-methylsulfonamidobenzylamino)cyclobut-3-ene-1,2-dione.

In other embodiments of these methods, the agent is an agonist of SGK1 that increases SGK1 activity. In such embodiments, the agonist of SGK1 activity is a an antibody or antigen-binding fragment thereof, a polypeptide, a small molecule, or an activating RNA molecule.

In some embodiments of these methods of modulating SGK1 activity, the subject has or is at risk for having a cancer. In some such embodiments, the cancer is selected from the group consisting of melanoma, skin cancer, precancerous skin lesions, breast cancer, prostate cancer, lung cancer, glioma, pancreatic cancer, head and neck cancer, renal cancer, sarcoma, ovarian cancer, rectal cancer, bladder cancer, mastocytoma, multiple myeloma, leukemia, lymphoma, cancer of the nervous system, bone cancer, bone marrow cancer, brain cancer, colon cancer, esophageal cancer, endometrial cancer, gastrointestinal cancer, genital-urinary cancer, gum cancer, retinal cancer, liver cancer, nasopharynx cancer, oral cancer, hematological neoplasm, follicular lymphoma, cervical cancer, osteosarcomas, thyroid cancer, testicular cancer, tongue cancer, and uterine cancer.

In some embodiments of these methods of modulating SGK1 activity, the subject has an infection. In some such embodiments, the infection is a viral, bacterial, yeast, or fungal infection.

In some embodiments, the bacterial infection is erysipelothricosis, listeriosis, anthrax, a Hemophilus infection, a Hemophilus influenza infection, Hemophilus ducreyi, Brucellosis, tularemia, bubonic plaque, pneumonic plague, septicemic plague, pestis minor, cat-scratch disease, a Pseudomonas infection, Campylobacter bacteria infection, cholera, infection with a Vibrio species, an Enterobacteriaceae infection, Klebsella pneumonia infection, typhoid fever, a nontyphoidal Salmonella infection, Shigellosis, a staphylococci infection, a group A streptococci infection, a group B streptococci infection, a groups C and G streptococci infection, a group D streptococci infection, an enterocooci infection, a pneumococci infection, pneumonia, thoracic empyema, bacterial meningitis, bacteremia, pneumococcal endocarditis, peritonitis, pneumococcal arthritis, otitis media, a meningococci infection, a Meningococci infection, a Neisseria gonorrhoeae infection, a Spirochetal infection, a Treponema infection, a Leptospirosis infection, a clostridia infection, a peptococci infection, a peptostreptococci infection, a Bacteroides fragilis infection, a Prevotella melaminogenica infection, a Fusobacterium infection, Mycobacterium tuberculosis, Mycobacterium bovis, Mycobacterium africanum, Mycobacterium leprae, Rickettsia typhi, Rickettsia rickettsii, Rickettsia prowazekii, Rickettsia-62 tsutsugamushi, Ehrlichiosis, Rickettsia akari, Coxiella burnetii, or Bartonella quintana.

In some embodiments, the viral infection is a respiratory viral infection, a Picornavirus, an Influenza virus, a respiratory syncytial virus, influenza A, influenza B, herpes simplex, herpes zoster, Epstein-Barr virus, cytomegalovirus, herpesvirus 6, human herpesvirus 7, herpesvirus 8, a central nervous system viral infection, polyomavirus infection of the brain, Tropical spastic paraparesis (HTLV-I), Arbovirus encephalitis, yellow fever, dengue fever, an Arenavirus infections, Lymphocytic choriomeningitis, a hemorrhagic fever, Bolivian hemorrhagic fever, Argentinean hemorrhagic fever, Lassa fever, Hantavirus infection, Ebola virus, Marburg viruses). human immunodeficiency virus (HIV), HIV-1, HIV-II virus, Hepatitis A, hepatitis B, hepatitis C, SARS, avian flu, papillomavirus.

In some embodiments, the fungal infection is ringworm, Trichophyton, Epidermophyton, groin ringworm, Microsporum, Candidiasis, Tinea versicolor, Histoplasmosis, Coccidioidomycosis, Blastomycosis, or Sporotrichosis.

In some embodiments of these methods of modulating SGK1 activity, the subject has or is at risk of having an atopic condition. In some such embodiments, the atopic condition is asthma, allergic rhinitis, gastrointestinal allergy, atopic dermatitis, eosinophilia, conjunctivitis, or eczema.

In one aspect, methods of modulating the differentiation of human TH17 cells from a population of human naïve CD4+ T cells are provided, the methods comprising contacting a human naïve CD4+ T cell with an effective amount of an agent that modulates serum and glucocorticoid-regulated kinase 1 (SGK1) activity.

In another aspect, methods of modulating the level of expression of IL-17 from human naïve CD4+ T cells are provided, such method comprising contacting a human naïve CD4+ T cell with an effective amount of an agent that modulates serum and glucocorticoid-regulated kinase 1 (SGK1) activity.

In another aspect, methods for modulating TH17 cell activity are provided, the methods comprising contacting a human naïve CD4+ T cell with an effective amount of an agent that modulates serum and glucocorticoid-regulated kinase 1 (SGK1) activity.

In another aspect, methods for modulating TH17 cell number are provided, the methods comprising contacting a human naïve CD4+ T cell with an effective amount of an agent that modulates serum and glucocorticoid-regulated kinase 1 (SGK1) activity.

In one aspect, methods of detecting TH17 cells in a test biological sample are provided. Such methods comprise contacting a test biological sample with a probe that detects a level of serum and glucocorticoid-regulated kinase 1 (SGK1) relative to a control biological sample, such that an increase in said level of SGK1 in the test biological sample relative to the control biological sample is indicative of the presence of TH17 cells in the test biological sample. In some embodiments of these methods, the biological sample is selected from the group consisting of blood sample, serum sample, cell sample, tissue sample, bone marrow and biopsy.

### DEFINITIONS

For convenience, certain terms employed herein, in the specification, examples and appended claims are collected here. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

The term “agent” as used herein means any compound or substance such as, but not limited to, a small molecule, nucleic acid, polypeptide, peptide, drug, ion, etc. An “agent” can be any chemical, entity, or moiety, including without limitation synthetic and naturally-occurring proteinaceous and non-proteinaceous entities. In some embodiments, an agent is a nucleic acid, a nucleic acid analogue, protein, antibody, peptide, aptame, oligomer of nucleic acids, amino acid, or carbohydrate, and includes, without limitation, proteins, oligonucleotides, ribozymes, DNAzymes, glycoproteins, siRNAs, lipoproteins, aptamers, and modifications and combinations thereof etc. In certain embodiments, agents are small molecules having a chemical moiety. For example, chemical moieties include unsubstituted or substituted alkyl, aromatic, or heterocyclyl moieties. Compounds can be known to have a desired activity and/or property, or can be selected from a library of diverse compounds.

As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The term “modulate” is used consistently with its use in the art, e.g., meaning to cause or facilitate a qualitative or quantitative change, alteration, or modification in one or more biological processes, mechanisms, effects, responses, functions, activities, pathways, or other phenomena of interest. Without limitation, such change may be an increase, decrease, or change in relative strength or activity of different components or branches of the process, mechanism, effect, response, function, activity, pathway, or phenomenon. Accordingly, as used herein “modulating” refers to a change of at least 10%, at least about 20%, at least about 30%, at least about 40%, at least about 50%, at least about 60%, at least about 70%, at least about 80%, at least about 90%, up to and including a 100% change, or any change of at least about 2-fold, at least about 3-fold, at least about 4-fold, at least about 5-fold, at least about 10-fold, at least about 100-fold, at least about 1000-fold, or any modulation between 2-fold and 1000-fold, or greater, as compared to a reference level. A “modulator” is an agent, such as a small molecule or other agents described herein, that causes or facilitates a qualitative or quantitative change, alteration, or modification in a process, mechanism, effect, response, function, activity, pathway, or phenomenon of interest.

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level or non-detectable level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2SD) difference relative to a reference. The term refers to statistical evidence that there is a difference. It is defined as the probability of making a decision to reject the null hypothesis when the null hypothesis is actually true. The decision is often made using the p-value.

As used herein, the term “DNA” is defined as deoxyribonucleic acid.

The term “polynucleotide” is used herein interchangeably with “nucleic acid” to indicate a polymer of nucleosides. Typically a polynucleotide of this invention is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (e.g. the succession of letters used as abbreviations for bases) that biochemically characterizes a specific nucleic acid. A polynucleotide sequence presented herein is presented in a 5′ to 3′ direction unless otherwise indicated.

The term “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a nonpolypeptide moiety covalently or noncovalently associated therewith is still considered a “polypeptide”. Exemplary modifications include glycosylation and palmitoylation. Polypeptides may be purified from natural sources, produced using recombinant DNA technology, synthesized through chemical means such as conventional solid phase peptide synthesis, etc. The term “polypeptide sequence” or “amino acid sequence” as used herein can refer to the polypeptide material itself and/or to the sequence information (e.g., the succession of letters or three letter codes used as abbreviations for amino acid names) that biochemically characterizes a polypeptide. A polypeptide sequence presented herein is presented in an N-terminal to C-terminal direction unless otherwise indicated.

The term “expression” refers to the cellular processes involved in producing RNA and proteins and as appropriate, secreting proteins, including where applicable, but not limited to, for example, transcription, translation, folding, modification and processing. “Expression products” include RNA transcribed from a gene, and polypeptides obtained by translation of mRNA transcribed from a gene. Expressing a cytokine, for example, can refer to RNA transcription of a cytokine, translation of a cytokine, secretion of a cytokine, processing of a cytokine, or any combination therein. Detecting expression of an RNA transcript or polypeptide can be performed using any method known to one of skill in the art, including, but not limited to, semi-quantitative and quantitative RT-PCR, Northern blot analysis, Western blot analysis, ELISA, bead arrays, chip arrays, and flow cytometry (including intracellular detection of proteins, such as cytokine, by flow cytometry).

As used herein, the term “target” refers to a biological molecule (e.g., SGK1 peptide, SGK1 polypeptide, protein, lipid, carbohydrate) to which an modulating agent, such as an inhibitor or an activator, can selectively bind. The target can be, for example, an intracellular target (e.g., an intracellular protein target) or a cell surface target (e.g., a membrane protein, a receptor protein).

As used herein, “selectively binds” or “specifically binds” refers to the ability of an activator or inhibitor, described herein, to bind to a target, such as the SGK1 polypeptide, with a K_{D} 10⁻⁵ M (10000 nM) or less, e.g., 10⁻⁶ M or less, 10⁻⁷ M or less, 10⁻⁸ M or less, 10⁻⁹M or less, 10⁻¹⁰ M or less, 10⁻¹¹ M or less, or 10⁻¹² M or less. For example, if an activator or inhibitor described herein binds to the SGK1 polypeptide with a K_{D} of 10⁻⁵ M or lower, but not to other molecules, or a related homologue, then the agent is said to specifically bind the SGK1 polypeptide. Specific binding can be influenced by, for example, the affinity and avidity of the activator or inhibitor and the concentration of the activator or inhibitor used. The person of ordinary skill in the art can determine appropriate conditions under which the activators or inhibitors described herein selectively bind using any suitable methods, such as titration of an activator or inhibitor in a suitable cell binding assay.

The term “specificity” refers to the number of different types of antigens or antigenic determinants to which a particular SGK1 modulating agent can bind. The specificity of an SGK1 modulating agent can be determined based on affinity and/or avidity. The affinity, represented by the equilibrium constant for the dissociation (K_{D}) of a target with an SGK1 modulating agent (such as a small molecule or antibody or antigen-binding fragment described herein), is a measure for the binding strength between the target and the SGK1 modulating agent: the lesser the value of the K_{D}, the stronger the binding strength between an antigenic determinant and the antigen-binding molecule. Alternatively, the affinity can also be expressed as the affinity constant (K_{A}), which is 1/K_{D}). As will be clear to the skilled person, affinity can be determined in a manner known per se, depending on the specific target of interest. Accordingly, an SGK1 modulating agent as defined herein is said to be “specific for” SGK1 compared to a second target when it binds to SGK1 with an affinity (as described above, and suitably expressed, for example as a K_{D} value) that is at least 10 times, such as at least 100 times, and preferably at least 1000 times, and up to 10.000 times or more better than the affinity with which the modulating agent binds to another target, such as SGK2.

As used herein, “immunoglobulin” refers to a family of polypeptides which retain the immunoglobulin fold characteristic of antibody molecules, which comprise two β sheets and, usually, a conserved disulphide bond. Members of the immunoglobulin superfamily are involved in many aspects of cellular and non-cellular interactions in vivo, including widespread roles in the immune system (for example, antibodies, T-cell receptor molecules and the like), involvement in cell adhesion (for example the ICAM molecules) and intracellular signaling (for example, receptor molecules, such as the PDGF receptor).

As used herein an “antibody” refers to IgG, IgM, IgA, IgD or IgE molecules or antigen-specific antibody fragments thereof (including, but not limited to, a Fab, F(ab′)₂, Fv, disulphide linked Fv, scFv, single domain antibody, closed conformation multispecific antibody, disulphide-linked scfv, diabody), whether derived from any species that naturally produces an antibody, or created by recombinant DNA technology; whether isolated from serum, B-cells, hybridomas, transfectomas, yeast or bacteria.

As described herein, an “antigen” is a molecule that is bound by a binding site on a polypeptide agent. Typically, antigens are bound by antibody ligands and are capable of raising an antibody response in vivo. An antigen can be a polypeptide, protein, nucleic acid or other molecule. In the case of conventional antibodies and fragments thereof, the antibody binding site as defined by the variable loops (L1, L2, L3 and H1, H2, H3) is capable of binding to the antigen. The term “antigenic determinant” refers to an epitope on the antigen recognized by an antigen-binding molecule (such as bispecific polypeptide agent described herein), and more particularly, by the antigen-binding site of said molecule.

As used herein, an “epitope” can be formed both from contiguous amino acids, or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5, about 9, or about 8-10 amino acids in a unique spatial conformation. An “epitope” generally includes the unit of structure conventionally bound by an immunoglobulin V_{H}/V_{L} pair, although it is recognized that, for example, a single domain antibody may only require a V_{H} or a V_{L} to recognize and bind to an antigen. Epitopes define the minimum binding site for an antibody, and thus represent the target of specificity of an antibody. The terms “antigenic determinant” and “epitope” can also be used interchangeably herein.

With respect to a target, the term “ligand interaction site” on the target means a site, epitope, antigenic determinant, part, domain or stretch of amino acid residues on the target that is a site for binding to a ligand, receptor or other binding partner, a catalytic site, a cleavage site, a site for allosteric interaction, a site involved in multimerisation (such as homomerization or heterodimerization) of the target or antigen; or any other site, epitope, antigenic determinant, part, domain or stretch of amino acid residues on the target or antigen that is involved in a biological action or mechanism of the target, i.e., SGK1. More generally, a “ligand interaction site” can be any site, epitope, antigenic determinant, part, domain or stretch of amino acid residues on the SGK1 polypeptide to which an inhibitor or agonist described herein can bind, such that SGK1 activity and/or expression is (and/or any pathway, interaction, signalling, biological mechanism or biological effect in which SGK1 is involved) is modulated.

An agent (such as a small molecule, an RNA interference molecule, an antibody, or generally an antigen binding protein or polypeptide or a fragment thereof) that can specifically bind to, that has affinity for, and/or that has specificity for a specific antigenic determinant, epitope, antigen or protein (or for at least one part, fragment or epitope thereof) is said to be “against” or “directed against” said antigenic determinant, epitope, antigen or protein.

An “RNA interference molecule” as used herein, is defined as any agent which interferes with or inhibits expression of a target gene or genomic sequence by RNA interference (RNAi). RNA interference involves the formation and activity of the RNA-induced silencing complex (RISC) (Gregory R I et al., 2005, Cell 123 (4): 631-640). Such RNA interfering agents include, but are not limited to, nucleic acid molecules including RNA molecules which are homologous to the SGK1 target gene or genomic sequence, or a fragment thereof, short interfering RNA (siRNA), short hairpin or small hairpin RNA (shRNA), microRNA (miRNA) and small molecules which interfere with or inhibit expression of the SGK1 target gene by RNA interference (RNAi).

The term “screening” as used herein refers to the use of cells, tissues, or derivatives of them in the laboratory to identify agents with a specific function, e.g., a modulating activity. In some embodiments, described herein are screening methods to identify agents (e.g., compounds or drugs) that inhibit or otherwise modulate SGK1 activity.

The term “library,” as used herein, refers to a mixture of heterogeneous agents, such as small molecules, polypeptides or nucleic acids. The library is composed of members, each of which have a single small molecule, polypeptide or nucleic acid sequence. To this extent, library is synonymous with repertoire. Structural and/or sequence differences between library members are responsible for the diversity present in the library. The library can take the form of a simple mixture of small molecules, polypeptides or nucleic acids, or can be in the form of organisms or cells, for example bacteria, viruses, animal or plant cells and the like, transformed with a library of, e.g., nucleic acids. Preferably, each individual organism or cell contains only one or a limited number of library members. Advantageously, the nucleic acids are incorporated into expression vectors, in order to allow expression of the polypeptides encoded by the nucleic acids. Therefore, in some embodiments, a library can take the form of a population of host organisms, each organism containing one or more copies of an expression vector containing a single member of the library in nucleic acid form which can be expressed to produce its corresponding polypeptide member. Thus, the population of host organisms has the potential to encode a large repertoire of genetically diverse polypeptide variants.

A “marker” as used herein is used to describe the characteristics and/or phenotype of a cell, e.g., a TH17 cell marker. Markers can be used for selection of cells comprising characteristics of interests. Markers will vary with specific cells. Markers are characteristics, whether morphological, functional or biochemical (enzymatic) characteristics of the cell of a particular cell type, or molecules expressed by the cell type. Preferably, such markers are proteins, and more preferably, possess an epitope for antibodies or other binding molecules available in the art. However, a marker may consist of any molecule found in or on the surface of a cell including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to express or produce one or more specific cytokines or chemokines, the ability to adhere to particular substrates, ability to incorporate or exclude particular dyes, ability to migrate under particular conditions, and the ability to differentiate along particular lineages. Markers may be detected by any method available to one of skill in the art. Markers can also be the absence of a morphological characteristic or absence of proteins, lipids etc. Markers can be a combination of a panel of unique characteristics of the presence and absence of polypeptides and other morphological characteristics. When a marker is a protein receptor or other such molecule expressed on the surface of a cell, it is termed herein as a “cell-surface marker.”

As used herein, an “immune response” refers to a response by a cell of the immune system, preferably a TH17 cell, but also including a B cell, T cell (CD4 or CD8), regulatory T cell, antigen-presenting cell, dendritic cell, monocyte, macrophage, NKT cell, NK cell, basophil, eosinophil, or neutrophil, to a stimulus. In some embodiments, the response is specific for a particular antigen (an “antigen-specific response”), and refers to a response by a CD4 T cell, such as a TH17 cell, CD8 T cell, or B cell, via their antigen-specific receptor. In some embodiments, an immune response is a T cell response, such as a CD4+ TH17 response. Such responses by these cells can include, for example, cytokine or chemokine production, proliferation, cytotoxicity, trafficking, or phagocytosis, and can be dependent on the nature of the immune cell undergoing the response.

As used herein, “T-cell trafficking” refers to migration of T lymphocytes, such as the TH17 cells described herein, to a site of an immune response. Naive T cells recirculate throughout the body, leaving and reentering the lymphoid tissues as they sample their environment for the presence of non-self antigens. Lymphoid tissues are specially adapted to help promote encounters between antigen-specific T-cell receptors expressed on T cells and their cognate antigens. Specialized antigen-presenting cells (APCs) concentrate within lymphoid tissues, and are specially adapted to interact with and to present antigens to T cells to initiate an immune response by T cells genetically programmed to recognize a particular antigen. Following T-cell activation in response to encounter with a specific antigen, T cells proliferate, undergo differentiation to produce a variety of secreted and cell-associated products, including cytokines, and migrate to tissue sites associated with the antigen. The result of this process is that naive T cells circulate randomly while activated T cells proliferate and home or traffic to specific tissue sites, i.e., sites of immune responses.

As used herein, a “site of an immune response” refers to any tissue or organ in which an immune response, as defined herein, is taking place. Such sites include lymphoid tissue and tissues in which immune cells develop and differentiate, such as lymph nodes (aortic, axillary, bronchopulmonary, buccal, celiac, cervical, cystic, deltopectoral, iliac, infraclavicular, inguinal, intercostal, internal thoracic, jugulodigastric, jugulo-omohyoid, lumbar, mastoid, mediastinal, mesenteric, occipital, para-aortic, pararectal, parotid, pectoral, popliteal, preaortic, pulmonary, retroauricular, retropharyngeal, submandibular, submental, subscapular, supratrochlear, tonsils, tracheobroncheal), mucosal associated lymphoid tissues (MALT) (e.g., Peyers's patches, gut associated lymphoid tissue (GALT), and lung associated lymphoid tissues), spleen, thymus, and bone marrow, but also includes other tissues or organs in or at which an immune response can take place, whether during an infection or during an autoimmune disease. Thus, a site of an immune response also includes brain or central nervous system (CNS), joint synovia, breast, lung, kidney, liver, pancreas (including pancreatic islets), stomach, intestine, ovary, uterus, testis, prostate, marrow, bone, muscle, and skin.

The term “anti-cancer therapy” refers to a therapy useful in treating cancer. Examples of anti-cancer therapeutic agents include, but are not limited to, e.g., surgery, chemotherapeutic agents, growth inhibitory agents, cytotoxic agents, agents used in radiation therapy, anti-angiogenesis agents, apoptotic agents, anti-tubulin agents, and among others, such as anti-HER-2 antibodies (e.g., Herceptin®), anti-CD20 antibodies, an epidermal growth factor receptor (EGFR) antagonist (e.g., a tyrosine kinase inhibitor), HER1/EGFR inhibitor (e.g., erlotinib (Tarceva®)), platelet derived growth factor inhibitors (e.g., Gleevec™ (Imatinib Mesylate)), a COX-2 inhibitor (e.g., celecoxib), interferons, cytokines, antagonists (e.g., neutralizing antibodies) that bind to one or more of the following targets ErbB2, ErbB3, ErbB4, PDGFR-beta, BlyS, APRIL, BCMA or VEGF receptor(s), TRAIL/Apo2, and other bioactive and organic chemical agents, etc. Combinations thereof are also specifically contemplated.

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents, and toxins such as small molecule toxins or enzymatically active toxins of bacterial, fungal, plant or animal origin, including fragments and/or variants thereof.

As used herein, the terms “chemotherapy” or “chemotherapeutic agent” refer to any chemical agent with therapeutic usefulness in the treatment of diseases characterized by abnormal cell growth. Such diseases include tumors, neoplasms and cancer as well as diseases characterized by hyperplastic growth. Chemotherapeutic agents as used herein encompass both chemical and biological agents. These agents function to inhibit a cellular activity upon which the cancer cell depends for continued survival. Categories of chemotherapeutic agents include alkylating/alkaloid agents, antimetabolites, hormones or hormone analogs, and miscellaneous antineoplastic drugs. Most if not all of these agents are directly toxic to cancer cells and do not require immune stimulation. In one embodiment, a chemotherapeutic agent is an agent of use in treating neoplasms such as solid tumors. In one embodiment, a chemotherapeutic agent is a radioactive molecule. One of skill in the art can readily identify a chemotherapeutic agent of use (e.g. see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2.sup.nd ed., COPYRGT. 2000 Churchill Livingstone, Inc; Baltzer L, Berkery R (eds): Oncology Pocket Guide to Chemotherapy, 2nd ed. St. Louis, Mosby-Year Book, 1995; Fischer D S, Knobf M F, Durivage H J (eds): The Cancer Chemotherapy Handbook, 4th ed. St. Louis, Mosby-Year Book, 1993).

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given either as a one-time treatment or at intervals of e.g., daily, twice a week, three times a week, weekly, or less frequently as judged by the administering clinician, and typical dosages range from 10 to 200 units (Grays) per day.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and the include plural references unless the context clearly dictates otherwise. Thus for example, references to “the method” includes one or more methods, and/or steps of the type described herein and/or which will become apparent to those persons skilled in the art upon reading this disclosure and so forth.

Other than in the operating examples, or where otherwise indicated, all numbers expressing quantities of ingredients or reaction conditions used herein should be understood as modified in all instances by the term “about.” The term “about” when used in connection with percentages can mean±1%.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology, and molecular biology can be found in The Merck Manual of Diagnosis and Therapy, 18th Edition, published by Merck Research Laboratories, 2006 (ISBN 0-911910-18-2); Robert S. Porter et al. (eds.), The Encyclopedia of Molecular Biology, published by Blackwell Science Ltd., 1994 (ISBN 0-632-02182-9); and Robert A. Meyers (ed.),

Molecular Biology and Biotechnology: a Comprehensive Desk Reference, published by VCH Publishers, Inc., 1995 (ISBN 1-56081-569-8); Immunology by Werner Luttmann, published by Elsevier, 2006. Definitions of common terms in molecular biology are found in Benjamin Lewin, Genes IX, published by Jones & Bartlett Publishing, 2007 (ISBN-13: 9780763740634); Kendrew et al. (eds.), The Encyclopedia of Molecular Biology, published by Blackwell Science Ltd., 1994 (ISBN 0-632-02182-9); and Robert A. Meyers (ed.), Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2 ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); or Methods in Enzymology: Guide to Molecular Cloning Techniques Vol. 152, S. L. Berger and A. R. Kimmerl Eds., Academic Press Inc., San Diego, USA (1987); Current Protocols in Molecular Biology (CPMB) (Fred M. Ausubel, et al. ed., John Wiley and Sons, Inc.), Current Protocols in Protein Science (CPPS) (John E. Coligan, et. al., ed., John Wiley and Sons, Inc.) and Current Protocols in Immunology (CPI) (John E. Coligan, et. al., ed. John Wiley and Sons, Inc.), which are all incorporated by reference herein in their entireties.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

### BRIEF DESCRIPTION OF THE FIGURES

FIG. 1 shows that TGF-β can upregulate the expression level of SGK1. FIG. 1 further demonstrates that addition of IL-6 together with TGF-β further increased the expression SGK1. IL-23, an IL-12 family cytokine, essential for enhancing generation of TH17 cells was found to further enhance the expression of SGK1.

FIG. 2 shows time-dependent expression of SGK1 during TH17 differentiation. Naïve CD4+ T cells were cultured in TH17 differentiating condition (TGF-β plus IL-6) for 96 hours. After 48 hours, TH17 cells were supplemented with IL-23 until the end of the culture, and it was found that expression of SGK1 was rapidly induced after 2 hours and dropped down to the base level after 8 hours, and that IL-23 further induced SGK1 expression.

FIG. 3 demonstrates that SGK1 expression is specifically critical for TH17 differentiation. Naïve CD4+ T cells from SGK1 deficient and wild-type mice were differentiated into TH1, TH2 and TH17 cells. It was found that upon stimulation with TGF-β and IL-6, wild-type T cells differentiates into TH17 cell (˜22%). However, SGK-deficient T cells showed significantly reduced IL-17 expression (˜12%). Importantly, TH1 and TH2 differentiation did not change.

FIG. 4 shows that SGK1 is essential for IL-23 dependent expansion of TH17 cells. Naïve CD4+ T cells were sorted from wild-type and SGK1-deficient mice and cultured under TH17 differentiation conditions. After a first round of stimulation, the cells were rested for two days in cytokine free medium. Two days later, cells were activated in the presence or absence of IL-23, and intracellular cytokine staining was performed for IL-17. IL-23 was able to expand already differentiated wild-type TH17 cells (11% to 13%), however SGK1-deficient TH17 cells failed to undergo IL-23 mediated expansion (˜4% to ˜1%).

FIG. 5 demonstrates that SGK1 is essential for IL-23 mediated expansion of TH17 cells. Wild-type and SGK1-deficient CD4+CD62L-cells (memory CD4 cells) were sorted and cultured with either anti-CD3 alone or anti-CD3 plus IL-23. IL-23 clearly enhanced the expression of IL-17A and IL-17F in wild-type cells, however SGK1-deficient memory cells were defective in inducing expression of IL-17A and IL-17F.

FIG. 6 demonstrates that inhibitors of PI3 kinase and AKT/MAP kinase do not influence SGK1 upregulation mediated by TGF-β and IL-6.

### DETAILED DESCRIPTION

Described herein are novel compositions and methods for modulating TH17 cell differentiation and activity via inhibition or activation of SGK1. These compositions and methods are based, in part, on the novel discovery by the inventors that SGK1, a serine/threonine kinase previously described as being involved in regulation of cellular sodium homeostasis, has a novel and unexpected function in the differentiation and function of a specific subset of CD4 T cells, the TH17 lineage, while not impacting the differentiation and function of other subsets of CD4 T cells, such as TH1 or TH2 cells. Accordingly, described herein are methods and compositions for modulation of TH17 cell differentiation, proliferation, activity, and/or function that rely upon modulating the activity or expression of SGK1. Such methods and compositions are useful in the treatment of a variety of disorders including autoimmune diseases, chronic inflammatory conditions, infectious diseases, and cancer.

### TH17 Cells

Distinct types of adaptive immune responses affording protection against different classes of pathogens are facilitated by the differentiation of CD4+ T cells into the corresponding types of effector T cells, which currently comprise TH1, TH2, and TH17 subsets. Through elaboration of distinct sets of cytokines and other soluble and cell-bound products, these cells act as immune effectors eliminating cells infected by pathogens. Importantly, such differentiated CD4+ T cells act as principal amplifiers and inducers of the appropriate inflammatory and effector responses in cells of the innate immune system and “nonimmune” cells. The amplified blocks of adaptive and innate immune responses lead to efficient clearance or containment of offending pathogens.

The downside of powerful mechanisms of protection against pathogen afforded by the immune system of higher organisms is inflammation associated with the “unwanted” immune responses against “self”, i.e., in autoimmune disorders, and environmental antigens and commensal microorganisms, i.e., in allergic and atopic disorders, as well as “collateral” damage to the host as a side effect of immune responses against pathogens. These side effects can be, at times, more devastating than the infection itself. TH17 cells have been implicated in numerous autoimmune diseases and other inflammatory conditions, and are most abundant at mucosal surfaces, particularly the intestinal lamina propria (LP).

Following infection with diverse microbes, T cells undergo differentiation when their TCRs are triggered in the presence of particular combinations of cytokines produced by innate immune cells ([Abbas et al., 1996] and [Mosmann and Coffman, 1989]). Infection of myeloid cells with intracellular bacteria and viruses typically elicits production of IL-12, which induces differentiation of interferon-γ (IFN-γ)-producing Th1 cells and cytotoxic CD8⁺ T cells that are best suited to clear such pathogens. Infection with parasitic worms, in contrast, induces production of IL-4 by cells of the innate immune system, and this, in turn, stimulates CD4⁺ T cells to differentiate into TH2 cells that produce more IL-4, as well as IL-5 and IL-13, cytokines involved in controlling expulsion of the helminths.

The third, recently described, subset of CD4 T helper cells, TH17 cells, are abundant at mucosal interfaces, where they contain infection with pathogenic bacteria and fungi (Weaver et al., 2007). These cells produce IL-17A (also referred to as IL-17), IL-17F, and IL-22, cytokines involved in neutrophilia, tissue remodeling and repair, and production of antimicrobial proteins.

TH17 cells differentiate in response to the STAT3-activating cytokines IL-6, IL-21, and IL-23, along with TGF-β and IL-1β (Korn et al., 2009). TH17 cells can also further comprise subsets of cells that produce IL-22, but not IL-17, such as skin-homing T helper cells that produce IL-22, but not IL-17 (Duhen et al., 2009). The differentiation of CD4⁺ T cells that produce IL-17 and IL-22 is influenced by the composition of the intestinal microbiota and by the presence of innate immune cells that amplify the TH17 cell response.

TH17 cells have been shown to differentiate in vitro from naive CD4⁺ T cells in response to TCR signaling in the presence of IL-6 and TGF-β, but not IL-23 (Bettelli et al., 2006 and Veldhoen et al., 2006). The receptor for IL-23 is expressed on naive murine CD4⁺ T cells only after stimulation in the presence of IL-6 or IL-21, and then these other cytokines can give way to the ability of IL-23 to stimulate continued differentiation of TH17 cells and, perhaps, their survival (Korn et al., 2007, Nurieva et al., 2007 and Zhou et al., 2007). In human T cells, IL-23R can be constitutively expressed on CD4⁺ T cells, and hence IL-17 expression can be induced by IL-23 in vitro (Manel et al., 2008). Mouse T cells bearing γδ TCRs, which are prominent in mucosal tissues, also express IL-23R constitutively and have been reported to differentiate into IL-17-producing cells early after exposure to IL-23 (Roark et al., 2008).

The “TH17 program,” as used herein refers to the expression of signature cytokines, the chemokine receptor CCR6, and IL-23R by a CD4+ TH17 cell. Some of these same features can also be found in other lymphoid cells, e.g., TCRγδ T cells, lymphoid tissue inducer (LTi) cells, and phenotypically related cells with NK cell markers, that secrete IL-17 and/or IL-22 (Colonna, 2009). These cells share with CD4+ TH17 cells the expression of the orphan nuclear receptor RORγt, which is both necessary and sufficient for expression of the genes that currently define the TH17 program (Ivanov et al., 2006). In addition to RORγt, other transcription factors have been shown to be required for the expression of IL-17 in polarized T helper cells, and several of these are also required for upregulation of RORγt upon polarization. These include IRF4 and BATF, whose expression is induced upon TCR signaling, and STAT3 (Brustle et al., 2007, Schraml et al., 2009, and Zhou and Littman, 2009). Additional transcription factors that contribute to the induction of IL-17 in polarized cells include, but are not limited to, Runx1/CBFβ, c-Maf, and the ligand-regulated aryl hydrocarbon receptor (AhR) (Bauquet et al., 2009, Veldhoen et al., 2008 and Zhang et al., 2008). RORα also contributes to some IL-17 expression in the absence of RORγt (Yang et al., 2008b). AhR has been shown to be required for induction of IL-22 in response to xenobiotic ligands. STAT3, IRF4, and BATF are required for expression of RORγt in TH17-polarized T helper cells, yet each contributes additionally, in cooperation with RORγt, to expression of IL-17 and, other key components of the TH17 program.

### SGK1

SGK1 (or serum/glucocorticoid-regulated kinase 1 or serine/threonine-protein kinase 1) is a serine/threonine protein kinase that has been shown to play an important role in cellular stress responses. This kinase activates certain potassium, sodium, and chloride channels, and is involved in the regulation of processes such as cell survival, neuronal excitability, and renal sodium excretion. High levels of expression of this gene have been thought to contribute to conditions such as hypertension and diabetic nephropathy. Several alternatively spliced transcript variants encoding different isoforms have been described for SGK1.

Accordingly, the term “SGK1” as used herein, refers to any of the following naturally occurring SGK1 isoforms having the amino acid sequence of:

MTVKTEAAKGTLTYSRMRGMVAILIAFMKQRRMGLNDFIQKIANNSYACKHPEVQSILKISQ PQEPELMNANPSPPPSPSQQINLGPSSNPHAKPSDFHFLKVIGKGSFGKVLLARHKAEEVFYA VKVLQKKAILKKKEEKHIMSERNVLLKNVKHPFLVGLHFSFQTADKLYFVLDYINGGELFYH LQRERCFLEPRARFYAAEIASALGYLHSLNIVYRDLKPENILLDSQGHIVLTDFGLCKENIEHN STTSTFCGTPEYLAPEVLHKQPYDRTVDWWCLGAVLYEMLYGLPPFYSRNTAEMYDNILNK PLQLKPNITNSARHLLEGLLQKDRTKRLGAKDDFMEIKSHVFFSLINWDDLINKKITPPFNPNV SGPNDLRHFDPEFTEEPVPNSIGKSPDSVLVTASVKEAAEAFLGFSYAPPTDSFL (SEQ ID NO:1), as described by, e.g., NP_{—}005618.2;
MVNKDMNGFPVKKCSAFQFFKKRVRRWIKSPMVSVDKHQSPSLKYTGSSMVHIPPGEPDFE SSLCQTCLGEHAFQRGVLPQENESCSWETQSGCEVREPCNHANILTKPDPRTFWTNDDPAFM KQRRMGLNDFIQKIANNSYACKHPEVQSILKISQPQEPELMNANPSPPPSPSQQINLGPSSNPH AKPSDFHFLKVIGKGSFGKVLLARHKAEEVFYAVKVLQKKAILKKKEEKHIMSERNVLLKNV KHPFLVGLHFSFQTADKLYFVLDYINGGELFYHLQRERCFLEPRARFYAAEIASALGYLHSLN IVYRDLKPENILLDSQGHIVLTDFGLCKENIEHNSTTSTFCGTPEYLAPEVLHKQPYDRTVDW WCLGAVLYEMLYGLPPFYSRNTAEMYDNILNKPLQLKPNITNSARHLLEGLLQKDRTKRLG AKDDFMEIKSHVFFSLINWDDLINKKITPPFNPNVSGPNDLRHFDPEFTEEPVPNSIGKSPDSVL VTASVKEAAEAFLGFSYAPPTDSFL (SEQ ID NO:2), as described by, e.g., NP_{—}001137148.1;
MSSQSSSLSEACSREAYSSHNWALPPASRSNPQPAYPWATRRMKEEAIKPPLKAFMKQRRM GLNDFIQKIANNSYACKHPEVQSILKISQPQEPELMNANPSPPPSPSQQINLGPSSNPHAKPSDF HFLKVIGKGSFGKVLLARHKAEEVFYAVKVLQKKAILKKKEEKHIMSERNVLLKNVKHPFL VGLHFSFQTADKLYFVLDYINGGELFYHLQRERCFLEPRARFYAAEIASALGYLHSLNIVYRD LKPENILLDSQGHIVLTDFGLCKENIEHNSTTSTFCGTPEYLAPEVLHKQPYDRTVDWWCLGA VLYEMLYGLPPFYSRNTAEMYDNILNKPLQLKPNITNSARHLLEGLLQKDRTKRLGAKDDF MEIKSHVFFSLINWDDLINKKITPPFNPNVSGPNDLRHFDPEFTEEPVPNSIGKSPDSVLVTASV KEAAEAFLGFSYAPPTDSFL (SEQ ID NO:3), as described by, e.g., NP_{—}001137149.1;
MGEMQGALARARLESLLRPRHKKRAEAQKRSESFLLSGLAFMKQRRMGLNDFIQKIANNSY ACKHPEVQSILKISQPQEPELMNANPSPPPSPSQQINLGPSSNPHAKPSDFHFLKVIGKGSFGKV LLARHKAEEVFYAVKVLQKKAILKKKEEKHIMSERNVLLKNVKHPFLVGLHFSFQTADKLY FVLDYINGGELFYHLQRERCFLEPRARFYAAEIASALGYLHSLNIVYRDLKPENILLDSQGHIV LTDFGLCKENIEHNSTTSTFCGTPEYLAPEVLHKQPYDRTVDWWCLGAVLYEMLYGLPPFYS RNTAEMYDNILNKPLQLKPNITNSARHLLEGLLQKDRTKRLGAKDDFMEIKSHVFFSLINWD DLINKKITPPFNPNVSGPNDLRHFDPEFTEEPVPNSIGKSPDSVLVTASVKEAAEAFLGFSYAPP TDSFL (SEQ ID NO:4), as described by, e.g., NP_{—}001137150.1; together with any other naturally occurring allelic variants, splice variants, and processed forms thereof. Typically, SGK1 refers to human SGK1. The term “SGK1” is also used to refer to truncated forms or fragments of the SGK1 polypeptide. Reference to any such forms of SGK1 can be identified in the application, e.g., by “SGK1 (102-426).” Specific residues of SGK1 can be referred to as, for example, “SGK1(125).”

SGK1 was described for the first time in 1993 as an immediate early gene in a rat mammary carcinoma cell line (Webster et al., 1993a; Webster et al., 1993b). It was shown in further studies that SGK-1 and its inducibility occurs in various cell lines and in cells of normal tissues (Brennan et al., 2000; Naray-Fejes-Toth et al., 2000; Cooper et al., 2001; Mikosz et al., 2001). SGK-1 belongs to a family of serine/threonine kinases of which to date three members are known and are referred to as SGK-1, SGK-2 and SGK-3/SGKL/CISK.

SGK-1 is expressed in virtually all tissues which have been tested to date, but the amounts of expressed mRNA can vary widely depending on the nature of the tissue type investigated (Gonzalez-Robayna et al., 1999; Waldegger et al., 1999; Alliston et al., 2000; Klingel et al., 2000; Lang et al., 2000; Loffing et al., 2001; Fillon et al., 2002; Warntges et al., 2002a;). In addition, SGK-1 mRNA is found in typical embryonic tissues. During mouse embryogenesis, SGK-1 mRNA shows developed-dynamic changes in specific tissues of the embryo (decidua, yolk sack, otic vesicle) and is detectable during organogenesis in lung buds, brain, heart, liver, thymus etc. (Lee et al., 2001).

A large number of stimuli have been shown to activate the transcription of SGK-1. These include, but are not limited to, mineralocorticoids (Brennan et al., 2000; Shigaev et al., 2000; Bhargava et al., 2001), gonadotropins (Richards et al., 1995; Gonzalez-Robayna et al., 2000), 1,25(OH)₂D₃ (Akutsu et al., 2001), p 53, osmotic, cell-volume and hypotonic changes (Waldegger et al., 1997; Klingel et al., 2000; Waldegger et al., 2000; Rozansky et al., 2002; Warntges et al., 2002a), cytokines such as GM-CSF and TNF-alpha (Cooper et al., 2001) or by TGF-beta (Kumar et al., 1999; Waldegger et al., 1999; Lang et al., 2000). Induction of SGK also takes place in further growth-dependent signaling pathways by serum (Webster et al., 1993a), insulin and IGF-1 (Kobayashi et al., 1999a; Park et al., 1999; Perrotti et al., 2001), FSH (Alliston et al., 1997), fibroblast- and platelet-derived growth factor (Davies et al., 2000), activators of the Erk signaling cascade (Hayashi et al., 2001) and TPA (Mizuno et al., 2001).

### Modulating TH17 Mediated Immune Responses Via SGK1

### SGK1 Inhibitors and SGK1 Activators

Described herein are novel therapeutic agents and methods for modulating TH17-mediated immune responses by inhibiting or activating SGK1 expression and/or activity. These therapeutic agents and uses thereof, and methods of modulating TH17 responses are based, in part, on the inventors' surprising discovery that the serine/threonine kinase SGK1, which had previously been primarily implicated in sodium regulation and blood pressure maintenance, has novel roles in the differentiation and maintenance of CD4⁺ TH17 cells. The inventors have discovered that in the absence of SGK1 expression, de novo TH17 differention from naïve CD4+ T cells in the presence of TGF-β and IL-6 is significantly impaired. Further, the inventors have found that the survival, maintenance, and stability of TH17 cells in the presence of IL-23 is significantly impaired in the absence of SGK1 expression. Furthermore, the inventors found that SGK1 expression is specific to regulation of TH17 cells, as no changes in Th1 and TH2 cell differentiation was observed in the absence of SGK1. Thus, SGK1 represents a novel target for specifically modulating TH17 responses.

Accordingly, described herein are therapeutic compositions, and methods of use thereof, for inhibiting SGK1 expression for the treatment of disorders mediated by dysregulated or increased TH17 cell activity, such as in the treatment of autoimmune disorders and other proinflammatory disorders. In other aspects, described herein are therapeutic compositions, and methods of use thereof, for activating or increasing SGK1 expression for the treatment of disorders in which increased TH17 cell activity and function provides therapeutic benefits, such as in infectious disorders.

Described herein are modulators of SGK1 activity and their use for the treatment of disorders and disease conditions whereby modulation of TH17 cell activity and/or function has beneficial effects and outcomes. Such SGK1 modulators include agents such as small molecules, nucleic acids, polypeptides, peptides, drugs, etc. An “SGK1 modulating agent” refers to any chemical, entity, or moiety, including without limitation synthetic and naturally-occurring proteinaceous (e.g., antibodies or antigen-binding fragments thereof) and non-proteinaceous (e.g., small molecule or nucleic acid-based) entities, that causes or facilitates a qualitative or quantitative change, alteration, or modification in one or more processes, mechanisms, effects, responses, functions, activities or pathways mediated by SGK1. Such changes mediated by an SGK1 modulating agent, such as an SGK1 inhibitor or an SGK1 activating agent described herein, can refer to a decrease or an increase in the activity or function of SGK1, such as a decrease or increase in, or inhibition or activation of, serine/threonine phosphorylation activity of SGK1, where, e.g., SGK1 enzymatic activity is assayed as described herein. Such modulating can, for example, also involve allosteric modulation of SGK1; and/or reducing or inhibiting the binding of SGK1 to one of its substrates or ligands, and/or competing with a natural ligand or substrate for binding to SGK1. Modulating can also involve activating the target or antigen or the mechanism or pathway in which it is involved. An SGK1 modulating agent can, for example, also cause or effect a change in respect to the folding or conformation of SGK1 (for example, upon binding of a ligand or interaction with a substrate), to associate with other (sub)units, or to disassociate from one or more subunits, or from a complex, such as an enzyme complex.

In some embodiments of the aspects described herein, an SGK1 modulating agent is a nucleic acid, a nucleic acid analogue, protein, antibody, peptide, aptamer, oligomer of nucleic acids, amino acid, or carbohydrate, and includes, without limitation, proteins, oligonucleotides, ribozymes, DNAzymes, glycoproteins, siRNAs, lipoproteins, aptamers, and modifications and combinations thereof. SGK1 agonists, activators, inhibitors, or antagonists can be naturally occurring and as a group, comprises synthetic ligands, small chemical molecules, antibodies or antigen-binding fragments thereof, polypeptides (e.g., dominant-negative SGK1 polypeptides), inhibitory RNA molecules (i.e., siRNA or antisense RNA), and the like. Such SGK1 modulating agents can be selected from compounds known to have a desired activity and/or property, or can be selected from a library of diverse compounds by screening methods, as known to one of skill in the art. For example, assays to identify SGK1 inhibitors and SGK1 activators include, e.g., applying or contacting putative SGK1 modulator compounds to cells, in the presence or absence of an SGK1 polypeptide or polynucleotide encoding SGK1, and then determining changes in expression or functional effects of the putative SGK1 modulator compound on the SGK1 polypeptide or polynucleotide encoding SGK1.

A variety of assays can be used to assay for SGK1 expression and/or activity. Transcript (mRNA) expression of SGK1 can be ascertained by any standard method known to one of skill in the art, such as Northern blot analysis, semi- or real-time quantitative PCR analyses, and nucleic acid-based high-throughput chip assays, such as microarrays. Protein expression of SGK1 can be determined using any standard method known to one of skill in the art, such as Western blot analysis, flow cytometric assays for intracellular molecules, kinase substrate assays, and the like.

The ability of an SGK1 modulating agent, i.e., SGK1 inhibitor or SGK1 activator, such as a small molecule or antibody or antigen-binding fragment thereof, to modulate SGK1 activity, such as kinase activity, can be ascertained using any of a variety of assays known to one of skill in the art. These include a variety of kinase assays known in the literature that can be readily performed by one of skill in the art as described in, for example, Dhanabal et al., Cancer Res. 59:189-197; Xin et al., J. Biol. Chem. 274:9116-9121; Sheu et al., Anticancer Res. 18:4435-4441; Ausprunk et al., Dev. Biol. 38:237-248; Gimbrone et al., J. Natl. Cancer Inst 52:413-427; Nicosia et al., In vitro 18:538-549.

For example, SGK1 or a fragment thereof comprising the kinase domain, can be expressed for the purposes of protein production in cells, such as insect cells, e.g., Sf21; S. frugiperda, and subsequently purified by affinity chromatography as a fusion protein with glutathione S-transferase in a baculovirus expression vector. The cultivation, infection and digestion of the cells as well as the purification of the fusion protein by column chromatography are carried out in accordance with manufacturer-oriented generic working instructions. Kinase activity is measured using various available measurement systems. In the scintillation proximity method (Sorg et al., J. of. Biomolecular Screening, 2002, 7, 11-19), the flashplate method or the filter binding test, the radioactive phosphorylation of a protein or peptide as substrate is measured using radioactively labelled ATP (³²P-ATP, ³³P-ATP). In the case of the presence of an inhibitory compound, a reduced radioactive signal, or none at all, can be detected. Furthermore, homogeneous time-resolved fluorescence resonance energy transfer (HTR-FRET) and fluorescence polarisation (FP) technologies, are useful as assay methods (Sills et al., J. of Biomolecular Screening, 2002, 191-214). Other non-radioactive ELISA assay methods use specific phospho-antibodies (phospho-ABs). Such phospho-antibodies only bind the phosphorylated substrate. Such binding can be detected by chemiluminescence using a second peroxidase-conjugated antibody (Ross et al., 2002, Biochem. J.). In another example, fluorescence polarization can be used to monitor binding of a putative SGK1 modulator to SGK1 and/or monitor SGK1 kinase activity.

In some aspects, the SGK modulating agents described herein are SGK1 inhibitors. As used herein, the terms “inhibitor of SGK1” or “SGK1 inhibitor” refer to an agent or compound that inhibits SGK1 signaling and downstream effector pathways, as those terms are used herein. In some embodiments of the aspects described herein, the downstream effector pathway inhibited by the SGK1 inhibitor is TH17 cell differentiation or a TH17 cell-activity mediated by SGK1 expression, and thus the SGK1 inhibitor is an inhibitor of TH17 cell differentiation or TH17 cell-activity. Thus, the term SGK1 inhibitor refers to an agent that: inhibits expression of an SGK1 polypeptide, including any of the polypeptides of SEQ ID NO:1-SEQ ID NO:4; inhibits expression of a polynucleotide encoding SGK1, including any polynucleotide sequence encoding any of the polynucleotides of SEQ ID NO:1-SEQ ID NO:4; or one that binds to, partially or totally blocks stimulation of, decreases, prevents, delays activation of, inactivates, desensitizes, or downregulates the activity of an SGK1 polypeptide or polynucleotide encoding SGK1. Such SGK1 inhibitors can e.g., inhibit SGK1 expression, e.g., SGK1 translation, post-translational processing of SGK1, stability, degradation, or nuclear or cytoplasmic localization of an SGK1 polypeptide, or transcription, post transcriptional processing, stability or degradation of a polynucleotide encoding SGK1, or bind to, partially or totally block stimulation of, or enzymatic (e.g., kinase) activity of SGK1. An SGK1 inhibitor can act directly or indirectly. SGK1 inhibition is achieved when the activity value of an SGK1 polypeptide, or a polynucleotide encoding SGK1 is about at least 10% less, and preferably, at least 20% less, at least 30% less, at least 40% less, at least 50% less, at least 60% less, at least 70% less, at least 80% less, at least 90% less, at least 95% less, or absent or undetectable in comparison to a reference or control level in the absence of the SGK1 inhibitor.

In some embodiments of these aspects, the SGK1 inhibitor is an antagonist. An SGK1 antagonist refers to an SGK1 inhibitor that does not provoke a biological response itself upon specifically binding to an SGK1 polypeptide or polynucleotide encoding SGK1, but blocks or dampens agonist-mediated or ligand-mediated responses, i.e., an SGK1 antagonist can bind, but does not activate, an SGK1 polypeptide or polynucleotide encoding SGK1, and the binding disrupts the interaction with an endogenous or exogenous SGK1 substrate, ligand, or agonist, displaces an endogenous or exogenous SGK1 substrate, ligand, or agonist, and/or inhibits the function of an SGK1 substrate, ligand, or agonist. SGK1 antagonists can mediate their effects by binding to, for example, the active site, i.e., enzymatic site, or to allosteric sites on an SGK1 polypeptide or a polynucleotide encoding SGK1.

In some embodiments of the aspects described herein, an SGK1 inhibitor is a small molecule of Formula (I):

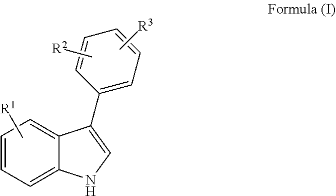

wherein R1 is optionally substituted phenyl, optionally substituted β-napthyl, or optionally substituted 3-CN-phenyl;
wherein R2 is CO₂R4 or C(R4,R5) CO₂R4;
wherein R3 and R4 are independently absent, H, C₁-C₆ alkyl, or C₅-C₈ cycloalkyl; each of which may be optionally substituted;
wherein R5 and R6 are independently absent, H, or C₁-C₆ alkyl, each of which may be optionally substituted; and
pharmaceutically acceptable salts thereof.

In some embodiments, the SGK1 inhibitor of Formula (I), is a small molecule of Formula (Ia):

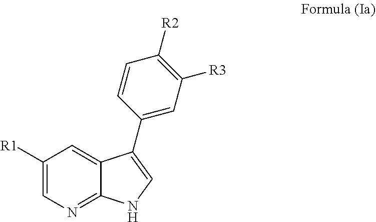

In some embodiments of Formula (Ia), R1 is phenyl, R2 is CO₂H, and R3 is H. In some embodiments, R1 is phenyl, R2 is CO₂, and R3 is

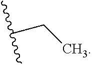 [SMILES: *CC]

In some embodiments, R1 is phenyl, R2 is CO₂H, and R3 is

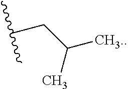 [SMILES: *CC(C)C]

In some embodiments, R1 is phenyl, R2 is CO₂H, and R3 is

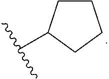 [SMILES: *C1CCCC1]

In some embodiments, R1 is β-napthyl, R2 is CH₂CO₂H, and R3 is H. In some embodiments, R1 is β-napthyl, R2 is

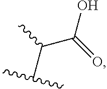 [SMILES: *C(C)C(=O)O]

and R3 is H. In some embodiments, R1 is β-napthyl, R2 is

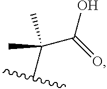

and R3 is H. In some embodiments, R1 is phenyl, R2 is

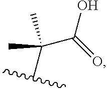

and R3 is H. In some embodiments, R1 is 3-CN-phenyl, R2 is

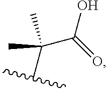

### and R3 is H.

In some embodiments of the aspects described herein, the small molecule SGK1 inhibitor is selected from the group consisting of 3-(4-hydroxy-3-methylphenylamino)-4-[(R)-1-(3-hydroxyphenyl)ethylamino]cyclobut-3-ene-1,2-dione; 3-(3-amino-1-tert-butyloxycarbonylindazol-5-ylamino)-4-(3-hydroxybenzylamino)cyclobut-3-ene-1,2-dione; 3-(3-amino-1-tert-butyloxycarbonylindazol-5-ylamino)-4-(3-methoxybenzylamino)cyclobut-3-ene-1,2-dione; 3-(3-amino-1H-indazol-5-ylamino)-4-(3-methoxybenzylamino)cyclobut-3-ene-1,2-dione; 3-(1H-indazol-5-ylamino)-4-[(R)-1-(3-methoxyphenyl)ethylamino]cyclo-but-3-ene-1,2-dione; 3-(1H-indazol-5-ylamino)-4-(3-methoxybenzylamino)cyclobut-3-ene-1,2-dione; 3-(1H-indazol-5-ylamino)-4-(3-hydroxybenzylamino)cyclobut-3-ene-1,2-dione; 3-(1-ethylaminocarbonylindazol-5-ylamino)-4-(3-methoxybenzylamino)cyclobut-3-ene-1,2-dione; 3-(1-ethylaminocarbonylindazol-5-ylamino)-4-(3-hydroxybenzylamino)cyclobut-3-ene-1,2-dione; 3-(3-amino-1H-indazol-5-ylamino)-4-[(R)-1-(3-methoxyphenyl)ethylamino]cyclobut-3-ene-1,2-dione; 3-(3-amino-1H-indazol-5-ylamino)-4-[(R)-1-(3-hydroxyphenyl)ethylamino]cyclobut-3-ene-1,2-dione; 3-(3-amino-1H-indazol-5-ylamino)-4-[(R)-1-(3-chlorophenyl)ethylamino]cyclobut-3-ene-1,2-dione; 3-(3-amino-1H-indazol-5-ylamino)-4-(3-chlorobenzylamino)cyclobut-3-ene-1,2-dione; 3-(3-amino-1H-indazol-5-ylamino)-4-(3-trifluoromethylbenzylamino)cyclobut-3-ene-1,2-dione; 3-(3-amino-1H-indazol-5-ylamino)-4-(3-trifluoromethoxybenzylamino)-cyclobut-3-ene-1,2-dione; 3-(3-amino-1H-indazol-5-ylamino)-4-(3-aminosulfonylbenzylamino)cyclobut-3-ene-1,2-dione; 3-(3-amino-1H-indazol-5-ylamino)-4-[(2-hydroxypyridin-4-ylmethyl)amino]cyclobut-3-ene-1,2-dione; 3-(3-amino-7-methyl-1H-indazol-5-ylamino)-4-[(R)-1-(3-methoxyphenyl)ethylamino]cyclobut-3-ene-1,2-dione; 3-(3-amino-7-methyl-1H-indazol-5-ylamino)-4-[(R)-1-(3-hydroxyphenyl)ethylamino]cyclobut-3-ene-1,2-dione; 3-(3-amino-7-methyl-1H-indazol-5-ylamino)-4-(3-aminosulfonylbenzylamino)cyclobut-3-ene-1,2-dione; 3-(3-amino-7-methyl-1H-indazol-5-ylamino)-4-[(2-hydroxypyridin-4-ylmethyl)amino]cyclobut-3-ene-1,2-dione; 3-(3-amino-7-methyl-1H-indazol-5-ylamino)-4-(3-methoxybenzylamino)-cyclobut-3-ene-1,2-dione; 3-(3-amino-7-methyl-1H-indazol-5-ylamino)-4-(3-hydroxybenzylamino)-cyclobut-3-ene-1,2-dione; 3-[3-(morpholin-4-yl)-1H-indazol-5-ylamino]-4-[(R)-1-(3-hydroxyphenyl)ethylamino]cyclobut-3-ene-1,2-dione; 3-[3-(piperidin-1-yl)-1H-indazol-5-ylamino]-4-[(R)-1-(3-hydroxyphenyl)ethylamino]cyclobut-3-ene-1,2-dione; 3-[3-(pyrrolidin-1-yl)-1H-indazol-5-ylamino]-4-[(R)-1-(3-hydroxyphenyl)ethylamino]cyclobut-3-ene-1,2-dione; 3-(3-bromo-1H-indazol-5-ylamino)-4-[(R)-1-(3-hydroxyphenyl)ethylamino]cyclobut-3-ene-1,2-dione; 3-(3-acetamido-1H-indazol-5-ylamino)-4-[(R)-1-(3-hydroxyphenyl)ethylamino]cyclobut-3-ene-1,2-dione; 3-(1H-indazol-5-ylamino)-4-[(R)-1-(3-hydroxyphenyl)ethylamino]cyclobut-3-ene-1,2-dione; 3-(7-bromo-1H-indazol-5-ylamino)-4-(3-methoxybenzylamino)cyclobut-3-ene-1,2-dione; 3-(7-bromo-1H-indazol-5-ylamino)-4-(3-hydroxybenzylamino)cyclobut-3-ene-1,2-dione; 3-(1H-indazol-5-ylamino)-4-(3-chlorobenzylamino)cyclobut-3-ene-1,2-dione; 3-(7-methyl-1H-indazol-5-ylamino)-4-(3-hydroxybenzylamino)cyclobut-3-ene-1,2-dione; 3-(7-methyl-1H-indazol-5-ylamino)-4-[(R)-1-(3-methoxyphenyl)ethylamino]cyclobut-3-ene-1,2-dione; 3-(7-methyl-1H-indazol-5-ylamino)-4-[(S)-1-(3-methoxyphenyl)ethylamino]cyclobut-3-ene-1,2-dione; 3-(7-methyl-1H-indazol-5-ylamino)-4-[(R)-1-(3-hydroxyphenyl)ethylamino]cyclobut-3-ene-1,2-dione; 3-(7-methyl-1H-indazol-5-ylamino)-4-(3-methoxybenzylamino)cyclobut-3-ene-1,2-dione; 3-(1H-indazol-5-ylamino)-4-(3-aminosulfonylbenzylamino)cyclobut-3-ene-1,2-dione; 3-(1H-indazol-5-ylamino)-4-[(R)-1-(3-hydroxyphenyl)-ethylamino]cyclobut-3-ene-1,2-dione; 3-(3-benzoylamino-1H-indazol-5-ylamino)-4-(3-hydroxybenzylamino)cyclobut-3-ene-1,2-dione; 3-(3-benzoylamino-1H-indazol-5-ylamino)-4-(3-methoxybenzylamino)cyclobut-3-ene-1,2-dione; 3-(3-benzoylamino-1H-indazol-5-ylamino)-4-[(R)-1-(3-methoxyphenyl)ethylamino]cyclobut-3-ene-1,2-dione; 3-(3-benzoylamino-1H-indazol-5-ylamino)-4-[(R)-1-(3-hydroxyphenyl)-ethylamino]cyclobut-3-ene-1,2-dione; 3-[3-(3-chlorobenzoylamino)-1H-indazol-5-ylamino]-4-[(R)-1-(3-meth-oxyphenyl)ethylamino]cyclobut-3-ene-1,2-dione; 3-[3-(3-chlorobenzoylamino)-1H-indazol-5-ylamino]-4-[(R)-1-(3-hydroxyphenyl)ethylamino]cyclobut-3-ene-1,2-dione; 3-[3-(3-chlorobenzoylamino)-1H-indazol-5-ylamino]-4-[(R)-1-(3-fluorophenyl)ethylamino]cyclobut-3-ene-1,2-dione; 3-[3-(3-chlorobenzoylamino)-1H-indazol-5-ylamino]-4-[(R)-1-(3-acet-amidophenyl)ethylamino]cyclobut-3-ene-1,2-dione; 3-[3-(3-chlorobenzoylamino)-1H-indazol-5-ylamino]-4-(3-methoxybenzylamino)cyclobut-3-ene-1,2-dione; 3-[3-(3-chlorobenzoylamino)-1H-indazol-5-ylamino]-4-(3-hydroxybenzylamino)cyclobut-3-ene-1,2-dione; 3-[3-(3-chlorobenzoylamino)-1H-indazol-5-ylamino]-4-(3-fluorobenzylamino)cyclobut-3-ene-1,2-dione; 3-[3-(3-chlorobenzoylamino)-1H-indazol-5-ylamino]-4-(3-acetamidobenzylamino)cyclobut-3-ene-1,2-dione; 3-(3-benzoylamino-1H-indazol-5-ylamino)-4-[(R)-1-(2,3-difluorophen-yl)ethylamino]cyclobut-3-ene-1,2-dione; 3-(3-benzoylamino-1H-indazol-5-ylamino)-4-[(R)-1-(3-methylsulfonamidophenyl)ethylamino]cyclobut-3-ene-1,2-dione; 3-[3-(3-chlorobenzoylamino)-1H-indazol-5-ylamino]-4-[(R)-1-(2,3-difluorophenyl)ethylamino]cyclobut-3-ene-1,2-dione; 3-[3-(3-chlorobenzoylamino)-1H-indazol-5-ylamino]-4-[(R)-1-(3-methylsulfonamidophenyl)ethylamino]cyclobut-3-ene-1,2-dione; 3-(3-benzoylamino-1H-indazol-5-ylamino)-4-(2,3-difluorobenzylamino-)cyclobut-3-ene-1,2-dione; 3-(3-benzoylamino-1H-indazol-5-ylamino)-4-(3-methylsulfonamidobenzylamino)cyclobut-3-ene-1,2-dione; 3-[3-(3-chlorobenzoylamino)-1H-indazol-5-ylamino]-4-(2,3-difluorob-enzylamino)cyclobut-3-ene-1,2-dione; and 3-[3-(3-chlorobenzoylamino)-1H-indazol-5-ylamino]-4-(3-methylsulfonamidobenzylamino)cyclobut-3-ene-1,2-dione.

In other embodiments of these aspects, the small molecule inhibitors of SGK1 can include, but are not limited to, the SGK1 antagonist GSK650394 (2-Cyclopentyl-4-(5-phenyl-1H-pyrrolo[2,3-b]pyridin-3-yl-benzoic acid) or derivatives thereof, described in Cancer Res 2008; 68(18):7475-83, the contents of which are herein incorporated in their entirety by reference; the small molecule indazolesquaric acid derivatives described in U.S. Patent Publication No.: US2009/0036449, the contents of which are herein incorporated in their entirety by reference; and the Acylhydrazone derivative and Pyridopyrimidine derivative SGK1 inhibitors described in U.S. Patent Publication No.: US2007/0191326 or in WO/2007/121963, the contents of which are herein incorporated in their entirety by reference. Other compounds of Formula (Ia) are described in WO2006/063167 and Bioorg. Med. Chem. Lett. 2009, 19, 4441-4445, the contents of which are herein incorporated in their entirety by reference.

For simplicity, chemical moieties as defined and referred to throughout can be univalent chemical moieties (e.g., alkyl, aryl, etc.) or multivalent moieties under the appropriate structural circumstances clear to those skilled in the art. For example, in some embodiments, an “alkyl” moiety can refer to a monovalent radical (e.g. CH₃—CH₂—), or in other embodiments, a bivalent linking moiety can be “alkyl,” in which case those skilled in the art will understand the alkyl to be a divalent radical (e.g., —CH₂—CH₂—), which is equivalent to the term “alkylene.” Similarly, in circumstances in which divalent moieties are required and are stated as being “alkoxy”, “alkylamino”, “aryloxy”, “alkylthio”, “aryl”, “heteroaryl”, “heterocyclic”, “alkyl” “alkenyl”, “alkynyl”, “aliphatic”, or “cycloalkyl”, those skilled in the art will understand that the terms “alkoxy”, “alkylamino”, “aryloxy”, “alkylthio”, “aryl”, “heteroaryl”, “heterocyclic”, “alkyl”, “alkenyl”, “alkynyl”, “aliphatic”, or “cycloalkyl” refer to the corresponding divalent moiety.

The term “halo” refers to any radical of fluorine, chlorine, bromine or iodine.

The term “acyl” refers to an alkylcarbonyl, cycloalkylcarbonyl, arylcarbonyl, heterocyclylcarbonyl, or heteroarylcarbonyl substituent, any of which may be further substituted by substituents. Exemplary acyl groups include, but are not limited to, (C₁-C₆)alkanoyl (e.g., formyl, acetyl, propionyl, butyryl, valeryl, caproyl, t-butylacetyl, etc.), (C₃-C₆)cycloalkylcarbonyl (e.g., cyclopropylcarbonyl, cyclobutylcarbonyl, cyclopentylcarbonyl, cyclohexylcarbonyl, etc.), heterocyclic carbonyl (e.g., pyrrolidinylcarbonyl, pyrrolid-2-one-5-carbonyl, piperidinylcarbonyl, piperazinylcarbonyl, tetrahydrofuranylcarbonyl, etc.), aroyl (e.g., benzoyl) and heteroaroyl (e.g., thiophenyl-2-carbonyl, thiophenyl-3-carbonyl, furanyl-2-carbonyl, furanyl-3-carbonyl, 1H-pyrroyl-2-carbonyl, 1H-pyrroyl-3-carbonyl, benzo[b]thiophenyl-2-carbonyl, etc.). In addition, the alkyl, cycloalkyl, heterocycle, aryl and heteroaryl portion of the acyl group may be any one of the groups described in the respective definitions.

The term “alkyl” refers to saturated non-aromatic hydrocarbon chains that may be a straight chain or branched chain, containing the indicated number of carbon atoms (these include without limitation methyl, ethyl, propyl, butyl, pentyl, hexanyl, which may be optionally inserted with N, O, S, SS, SO₂, C(O), C(O)O, OC(O), C(O)N or NC(O). For example, C₁-C₆ indicates that the group may have from 1 to 6 (inclusive) carbon atoms in it.

The term “alkenyl” refers to an alkyl that comprises at least one double bond. Exemplary alkenyl groups include, but are not limited to, for example, ethenyl, propenyl, butenyl, 1-methyl-2-buten-1-yl and the like.

The term “alkynyl” refers to an alkyl that comprises at least one triple bond.

The term “alkoxy” refers to an —O-alkyl radical.

The term “aminoalkyl” refers to an alkyl substituted with an amino.

The term “aryl” refers to monocyclic, bicyclic, or tricyclic aromatic ring system wherein 0, 1, 2, 3, or 4 atoms of each ring may be substituted by a substituent. Exemplary aryl groups include, but are not limited to, phenyl, naphthyl, anthracenyl, azulenyl, fluorenyl, indanyl, indenyl, naphthyl, phenyl, tetrahydronaphthyl, and the like.

The term “arylalkyl” refers to alkyl substituted with an aryl or aryl substituted with an alkyl.

The term “cycloalkyl” refers to saturated and partially unsaturated cyclic hydrocarbon groups having 3 to 12 carbons, for example, 3 to 8 carbons, and, for example, 3 to 6 carbons, wherein the cycloalkyl group additionally may be optionally substituted. Exemplary cycloalkyl groups include, but are not limited to, cyclopropyl, cyclobutyl, cyclopentyl, cyclopentenyl, cyclohexyl, cyclohexenyl, cycloheptyl, cyclooctyl, and the like.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent. Exemplary heteroaryl groups include, but are not limited to, pyridyl, furyl or furanyl, imidazolyl, benzimidazolyl, pyrimidinyl, thiophenyl or thienyl, pyridazinyl, pyrazinyl, quinolinyl, indolyl, thiazolyl, naphthyridinyl, and the like.

The term “heteroarylalkyl” refers to an alkyl substituted with a heteroaryl.

The term “heterocyclyl” refers to a non-aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Exemplary heterocyclyl groups include, but are not limited to piperazinyl, pyrrolidinyl, dioxanyl, morpholinyl, tetrahydrofuranyl, and the like.

The term “haloalkyl” refers to an alkyl group having one, two, three or more halogen atoms attached thereto. Exemplary haloalkyl groups include, but are not limited to chloromethyl, bromoethyl, trifluoromethyl, and the like.

The term “optionally substituted” means that the specified group or moiety, such is unsubstituted or is substituted with one or more (typically 1-4 substituents) independently selected from the group of substituents listed herein in the definition for “substituents” or otherwise specified. The substituents may be “separate” substituents, for instance, a halo group and an alkoxy groups bonded to different carbon atoms in a benzene ring, or the substituents may be “stacked” on one another, for instance, an acyl group (such as formyl) that is substituted with an aminosulfonyl group that is substituted with an arylalkyl (such as toluene).

The term “substituents” refers to a group that replaces a hydrogen at any atom of the substituted group or moiety, as well as a group “substituted” on an alkyl, alkenyl, alkynyl, cycloalkyl, aryl, heterocyclyl, heteroaryl, acyl, amino group at any atom of that group. Suitable substituents include, without limitation, halo, hydroxy, oxo, nitro, haloalkyl, alkyl, alkenyl, alkynyl, alkaryl, aryl, aralkyl, alkoxy, aryloxy, amino, aminosulfonyl, acylamino, alkylcarbanoyl, arylcarbanoyl, aminoalkyl, alkoxycarbonyl, carboxy, hydroxyalkyl, alkylthio, CF₃, N-morphilino, phenylthio, alkanesulfonyl, arenesulfonyl, alkanesulfonamido, arenesulfonamido, aralkylsulfonamido, alkylcarbonyl, acyloxy, cyano or ureido. In some cases, two substituents, together with the carbons to which they are attached to can form a ring.

In many cases, protecting groups are used during preparation of the compounds of the invention. As used herein, the term “protected” means that the indicated moiety has a protecting group appended thereon. In some preferred embodiments of the invention, compounds contain one or more protecting groups. A wide variety of protecting groups can be employed in the methods of the invention. In general, protecting groups render chemical functionalities inert to specific reaction conditions, and can be appended to and removed from such functionalities in a molecule without substantially damaging the remainder of the molecule.

Representative hydroxyl protecting groups, for example, are disclosed by Beaucage et al. (Tetrahedron 1992, 48, 2223-2311). Further hydroxyl protecting groups, as well as other representative protecting groups, are disclosed in Greene and Wuts, Protective Groups in Organic Synthesis, Chapter 2, 2d ed., John Wiley & Sons, New York, 1991, and Oligonucleotides And Analogues A Practical Approach, Ekstein, F. Ed., IRL Press, N.Y, 1991. Examples of hydroxyl protecting groups include, but are not limited to, t-butyl, t-butoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, 2,6-dichlorobenzyl, diphenylmethyl, p,p′-dinitrobenzhydryl, p-nitrobenzyl, triphenylmethyl, trimethylsilyl, triethylsilyl, t-butyldimethylsilyl, t-butyldiphenylsilyl, triphenylsilyl, benzoylformate, acetate, chloroacetate, trichloroacetate, trifluoroacetate, pivaloate, benzoate, p-phenylbenzoate, 9-fluorenylmethyl carbonate, mesylate and tosylate.

Nitrogen- or amino-protecting groups stable to acid treatment are selectively removed with base treatment, and are used to make reactive amino groups selectively available for substitution. Exemplary amino-protecting groups include, but are not limited to, carbamate protecting groups, such as 2-trimethylsilylethoxycarbonyl (Teoc), 1-methyl-1-(4-biphenylyl)ethoxycarbonyl (Bpoc), t-butoxycarbonyl (BOC), allyloxycarbonyl (Alloc), 9-fluorenylmethyloxycarbonyl (Fmoc), and benzyloxycarbonyl (Cbz); amide protecting groups, such as formyl, acetyl, trihaloacetyl, benzoyl, and nitrophenylacetyl; sulfonamide protecting groups, such as 2-nitrobenzenesulfonyl; and imine and cyclic imide protecting groups, such as phthalimido and dithiasuccinoyl.

In other embodiments of these aspects, expression of SGK1 is suppressed or inhibited by RNA interference using, e.g., dsRNA, ssRNA, siRNA, miRNA, artificial derivatives of the foregoing, and the like. Exemplary SGK1 inhibitors using RNA interference mechanisms to inhibit SGK1 expression, include, but are not limited to, GUCCUUCUCAGCAAAUCAAUU (sense; SEQ ID NO:5); UUGAUUUGCUGAGAAGGACUU (antisense; SEQ ID NO:6); and SGK1 specific siRNAs, shRNA plasmids, and shRNA lentiviral particle commercially available from Santa Cruz Biotechnology, Inc.

In other embodiments, an SGK1 inhibitor comprises an anti-SGK1 antibody or an antigen-binding fragment thereof. When SGK1-specific antibodies or antigen-binding fragments thereof are used in inhibiting SGK1 activity and/or expression, it is understood that the antibody or antigen-binding fragment thereof is a “blocking” antibody or an antibody “antagonist,” i.e., it is one that inhibits or reduces biological activity of SGK1 upon binding, and does not activate or promote SGK1 signaling. For example, an SGK1 antagonist antibody can bind SGK1 and inhibit the ability of SGK1 to, for example, phosphorylate serine or threonine on a enzymatic substrate. In certain embodiments, the blocking antibodies or antagonist antibodies or fragments thereof described herein completely inhibit the biological activity of SGK1. Such anti-SGK1 antibodies include all such classes, subclasses and types of human antibody species. For example, as used herein, antibodies to SGK1 polypeptides also include antibodies to fusion proteins comprising SGK1 polypeptides or fragments of SGK1 polypeptides. More specifically, the SGK1 inhibitor can be a monoclonal or single specificity anti-SGK1 antibody or antigen-binding fragment thereof. The anti-SGK1 antibody or antigen-binding fragment thereof may be human, humanized, chimeric, or an in vitro generated antibody to human SGK1, as described herein. In addition, anti-SGK1 antibodies are available commercially, e.g., from R&D Systems, Abcam, and Santa Cruz Biotechnology, Inc.

Accordingly, in some embodiments of these aspects, the anti-SGK1 blocking antibody or antigen-binding fragment thereof is a human SGK1-specific antibody fragment. In some embodiments, the anti-SGK1 blocking antibody fragment is a Fab fragment comprising V_{L}, C_{L}, V_{H} and C_{H}1 domains. In some embodiments, the anti-SGK1 blocking antibody or antigen-binding fragment thereof is a Fab′ fragment, which is a Fab fragment having one or more cysteine residues at the C-terminus of the C_{H}1 domain. In some embodiments, the anti-SGK1 blocking antibody or antigen-binding fragment thereof is a Fd fragment comprising V_{H} and C_{H}1 domains. In some embodiments, the anti-SGK1 blocking antibody or antigen-binding fragment thereof is a Fd′ fragment comprising V_{H} and C_{H}1 domains and one or more cysteine residues at the C-terminus of the C_{H}1 domain. In some embodiments, the anti-SGK1 blocking antibody or antigen-binding fragment thereof is a Fv fragment comprising the V_{L} and V_{H} domains of a single arm of an antibody. In some embodiments, the anti-SGK1 blocking antibody or antigen-binding fragment thereof is a dAb fragment comprising a V_{H} domain or a V_{L} domain. In some embodiments, the anti-SGK1 blocking antibody or antigen-binding fragment thereof comprises isolated CDR regions. In some embodiments, the anti-SGK1 blocking antibody or antigen-binding fragment thereof is a F(ab′)₂ fragment, which comprises a bivalent fragment comprising two Fab′ fragments linked by a disulphide bridge at the hinge region. In some embodiments, the anti-SGK1 blocking antibody or antigen-binding fragment thereof is a single chain antibody molecule, such as a single chain Fv. In some embodiments, the anti-SGK1 blocking antibody or antigen-binding fragment thereof is a diabody comprising two antigen binding sites, comprising a heavy chain variable domain (V_{H}) connected to a light chain variable domain (V_{L}) in the same polypeptide chain. In some embodiments, the anti-SGK1 blocking antibody or antigen-binding fragment thereof is a linear antibody comprising a pair of tandem Fd segments (V_{H}-C_{H}1-V_{H}-C_{H}1) which, together with complementary light chain polypeptides, form a pair of antigen binding regions.

In other aspects, the SGK modulating agents described herein are SGK1 activators. As used herein, the terms “SGK1 activator,” “activator of SGK1,” and “SGK1 agonist” refer to an agent that binds to an SGK1 polypeptide or polynucleotide encoding SGK1, and stimulates, increases or upregulates expression of, or enhances enzymatic (serine/threonine kinase) activity of an SGK1 polypeptide or polynucleotide encoding SGK1. An increase in SGK1 activity or SGK1 expression is achieved by an SGK1 activator when the activity of or expression of an SGK1 polypeptide or a polynucleotide encoding SGK1 is at least 10% higher, at least 20% higher, at least 30% higher, at least 40% higher, at least 50% higher, at least 60% higher, at least 70% higher, at least 80% higher, at least 90%, at least 100% higher, at least 2-fold higher, at least 3-fold higher, at least 5-fold higher, at least 10-fold higher, at least 15-fold higher, at least 25-fold higher, at least 50-fold higher, at least 100-fold higher, at least 1000-fold higher, or more, relative to a reference activity or expression of an SGK1 polypeptide or polynucleotide encoding SGK1 in the absence of the SGK1 activator.

In some embodiments of these aspects, the SGK1 activator or agonist is an antibody or antigen-binding fragment thereof, a polypeptide, a small molecule, or an activating nucleic acid molecule, such as an activating RNA molecule.

When SGK1-specific antibodies or antigen-binding fragments thereof are used in activating SGK1 activity and/or expression, it is understood that the antibody or antigen-binding fragment thereof is an “activating” antibody or an antibody “agonist,” i.e., it is one that increases or promotes biological activity of SGK1, such as promoting TH17 differentiation or TH17 cell activity upon binding. For example, an SGK1 activating antibody can bind SGK1 and promote or increase the ability of SGK1 to, for example, phosphorylate serine or threonine on an enzymatic substrate. Such anti-SGK1 activating antibodies include all such classes, subclasses and types of human antibody species. Such activating antibodies to SGK1 polypeptides also include antibodies to fusion proteins comprising SGK1 polypeptides or fragments of SGK1 polypeptides. The SGK1 activating antibody can be a monoclonal or single specificity anti-SGK1 antibody or antigen-binding fragment thereof. The anti-SGK1 activating antibody or antigen-binding fragment thereof may be human, humanized, chimeric, or an in vitro generated activating antibody to human SGK1, as described herein.

Accordingly, in some embodiments of these aspects, the anti-SGK1 activating antibody or antigen-binding fragment thereof is a human SGK1-specific antibody fragment. Activating antibodies or antigen-binding fragments thereof can take any of the forms for antibodies or antigen-binding fragments thereof described herein in the context of antagonist or inhibitory antibodies.

In some embodiments, an SGK1 activator indirectly activates SGK1 via, for example, activation of an upstream regulator of SGK1, such as PPARγ. Thus, in some embodiments, an SGK1 activator is a PPARγ agonist, such as pioglitazone and L-805645.

### Production of Anti-SGK1 Antibodies and Antigen-Binding Fragments Thereof

Non-limiting methods of producing the anti-SGK1 blocking and agonist antibodies for use in the compositions and methods described herein are detailed below.

Polyclonal Antibodies.

Polyclonal antibodies are preferably raised in animals by multiple subcutaneous (sc) or intraperitoneal (ip) injections of the relevant antigen, e.g., SGK1, and an adjuvant. It can be useful, in some embodiments, to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Monoclonal Antibodies.

Various methods for making monoclonal antibodies specific for SGK1 as described herein are available in the art. For example, the monoclonal antibodies can be made using the hybridoma method first described by Kohler et al., Nature, 256:495 (1975), or by recombinant DNA methods (U.S. Pat. No. 4,816,567).

In the hybridoma method, a mouse or other appropriate host animal, is immunized to elicit lymphocytes that produce or are capable of producing antibodies that will specifically bind to the SGK1 protein or fragment thereof used for immunization. Alternatively, lymphocytes can be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)). The hybridoma cells thus prepared are seeded and grown in a suitable culture medium that preferably contains one or more substances that inhibit the growth or survival of the unfused, parental myeloma cells. Preferred myeloma cells are those that fuse efficiently, support stable high-level production of antibody by the selected antibody-producing cells, and are sensitive to a medium such as HAT medium. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)). Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA). After hybridoma cells are identified that produce antibodies of the desired specificity, affinity, and/or activity, the clones can be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells can be grown in vivo as ascites tumors in an animal. DNA encoding the monoclonal antibodies can be readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA can be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells.

In some embodiments, antibodies or antibody fragments that specifically bind SGK1 can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The DNA sequences encoding the antibodies or antibody fragment that specifically bind SGK1 also can be modified, for example, by substituting the coding sequence for human heavy- and light-chain constant domains in place of the homologous murine sequences (U.S. Pat. No. 4,816,567; Morrison, et al., Proc. Natl. Acad. Sci. USA, 81:6851 (1984)), or by covalently joining to the immunoglobulin coding sequence all or part of the coding sequence for a non-immunoglobulin polypeptide.

Such non-immunoglobulin polypeptides can be substituted for the constant domains of an antibody, or they can be substituted for the variable domains of one antigen-combining site of an antibody to create a chimeric bivalent antibody comprising one antigen-combining site having specificity for an antigen and another antigen-combining site having specificity for a different antigen.

Humanized and Human Antibodies.

A humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody.

The choice of human variable domains, both light and heavy, to be used in making the humanized antibodies is very important to reduce antigenicity. According to the so-called “best-fit” method, the sequence of the variable domain of a rodent antibody, is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987)). Another method uses a particular framework derived from the consensus sequence of all human antibodies of a particular subgroup of light or heavy chains. The same framework can be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties, for example, the ability to inhibit SGK1 activity and/or function. To achieve this goal, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the CDR residues are directly and most substantially involved in influencing antigen binding.

Alternatively, it is possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and Duchosal et al. Nature 355:258 (1992).

Alternatively, phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats; for their review see, e.g., Johnson, Kevin S, and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905.

Human antibodies can also be generated by in vitro activated B cells (see U.S. Pat. Nos. 5,567,610 and 5,229,275).

Antibody Fragments.

In some embodiments of the aspects described herein, an antibody specific for SGK1 can be treated or processed into an antibody fragment thereof. Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185.

### Pharmaceutical Formulations of SGK1 Inhibitors and SGK1 Activators

Therapeutic formulations of the SGK1 inhibitors and SGK1 activators described herein can be prepared, in some aspects, by mixing an SGK1 inhibitor, such as a small molecule SGK1 inhibitor of Formula (I) or Formula (Ia), blocking or inhibitory SGK1 antibody or antigen-binding fragment thereof, or nucleic acid-based SGK1 inhibitor, or an SGK1 activator described herein, having the desired degree of purity with one or more pharmaceutically acceptable carriers, excipients or stabilizers (Remington's Pharmaceutical Sciences 16th edition, Osol, A. Ed. (1980)), in the form of lyophilized formulations or aqueous solutions. Such therapeutic formulations of the SGK1 inhibitors and SGK1 activators described herein include formulation into pharmaceutical compositions or pharmaceutical formulations for parenteral administration, e.g., intravenous; mucosal, e.g., intranasal; enteral, e.g., oral; topical, e.g., transdermal; ocular, or other mode of administration.

As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” as used herein means a pharmaceutically acceptable material, composition or vehicle, such as a liquid or solid filler, diluent, excipient, solvent, media, encapsulating material, manufacturing aid (e.g., lubricant, talc magnesium, calcium or zinc stearate, or steric acid), or solvent encapsulating material, involved in maintaining the activity, function of, solubility of, and/or stability of a SGK1 modulator as described herein.

Some non-limiting examples of acceptable carriers, excipients, or stabilizers that are nontoxic to recipients at the dosages and concentrations employed, include pH buffered solutions such as phosphate, citrate, and other organic acids; antioxidants, including ascorbic acid and methionine; lubricating agents, such as magnesium stearate, sodium lauryl sulfate and talc; excipients, such as cocoa butter and suppository waxes; oils, such as peanut oil, cottonseed oil, safflower oil, sesame oil, olive oil, corn oil and soybean oil; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, HDL, LDL, or immunoglobulins; hydrophilic polymers, such as polyvinylpyrrolidone; amino acids such as glycine, glutamine, asparagine, histidine, arginine, or lysine; monosaccharides, disaccharides, and other carbohydrates including mannose, starches (corn starch or potato starch), or dextrins; cellulose, and its derivatives, such as sodium carboxymethyl cellulose, methylcellulose, ethyl cellulose, microcrystalline cellulose and cellulose acetate; chelating agents such as EDTA; sugars such as sucrose, glucose, lactose, mannitol, trehalose or sorbitol; salt-forming counter-ions such as sodium; metal complexes (e.g. Zn-protein complexes); glycols, such as propylene glycol; polyols, such as glycerin; esters, such as ethyl oleate and ethyl laurate; agar; buffering agents, such as magnesium hydroxide and aluminum hydroxide; alginic acid; pyrogen-free water; isotonic saline; Ringer's solution; polyesters, polycarbonates and/or polyanhydrides; C2-C12 alcohols, such as ethanol; powdered tragacanth; malt; and/or non-ionic surfactants such as TWEEN™, PLURONICS™ or polyethylene glycol (PEG); and/or other non-toxic compatible substances employed in pharmaceutical formulations. Wetting agents, coloring agents, release agents, coating agents, sweetening agents, flavoring agents, perfuming agents, preservative and antioxidants can also be present in the formulation.

In some embodiments, a therapeutic formulation comprising an SGK1 inhibitor or an SGK1 activator comprises a pharmaceutically acceptable salt, typically, e.g., sodium chloride, and preferably at about physiological concentrations. Optionally, the formulations described herein can contain a pharmaceutically acceptable preservative. In some embodiments, the preservative concentration ranges from 0.1 to 2.0%, typically v/v. Suitable preservatives include those known in the pharmaceutical arts. Benzyl alcohol, phenol, m-cresol, methylparaben, and propylparaben are examples of preservatives. Optionally, the formulations described herein can include a pharmaceutically acceptable surfactant at a concentration of 0.005 to 0.02%.

In some embodiments, an SGK1 modulator, such as an SGK1 inhibitor or an SGK1 activator described herein, can be specially formulated for administration of the compound to a subject in solid, liquid or gel form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, an SGK1 modulator, such as an SGK1 inhibitor or an SGK1 activator described herein, can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960. Examples of dosage forms include, but are not limited to: tablets; caplets; capsules, such as hard gelatin capsules and soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquids such as suspensions (e.g., aqueous or non-aqueous liquid suspensions, oil-in-water emulsions, or water-in-oil liquid emulsions), solutions, and elixirs; and sterile solids (e.g., crystalline or amorphous solids) that can be reconstituted to provide liquid dosage forms.

In some embodiments, parenteral dosage forms of the SGK1 modulators, such as SGK1 inhibitors or SGK1 activators described herein, can be administered to a subject in need of treatment, such as a subject having an autoimmune disorder, or a subject having an infectious disease, by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, controlled-release parenteral dosage forms, and emulsions.

Suitable vehicles that can be used to provide parenteral dosage forms described herein are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Due to their ease of administration, tablets and capsules represent the most advantageous solid oral dosage unit forms, in which case solid pharmaceutical excipients are used. If desired, tablets can be coated by standard aqueous or nonaqueous techniques. These dosage forms can be prepared by any of the methods of pharmacy. In general, pharmaceutical compositions and dosage forms are prepared by uniformly and intimately admixing the active ingredient(s) with liquid carriers, finely divided solid carriers, or both, and then shaping the product into the desired presentation if necessary.

Typical oral dosage forms of the compositions are prepared by combining the pharmaceutically acceptable salt of an SGK1 modulator, such as an SGK1 inhibitor or an SGK1 activator described herein, in an intimate admixture with at least one excipient according to conventional pharmaceutical compounding techniques. Excipients can take a wide variety of forms depending on the form of the composition desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, microcrystalline cellulose, kaolin, diluents, granulating agents, lubricants, binders, and disintegrating agents.

Binders suitable for use in the pharmaceutical formulations described herein include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Examples of fillers suitable for use in the pharmaceutical formulations described herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions described herein is typically present in from about 50 to about 99 weight percent of the pharmaceutical composition.

Disintegrants are used in the oral pharmaceutical formulations described herein to provide tablets that disintegrate when exposed to an aqueous environment. A sufficient amount of disintegrant that is neither too little nor too much to detrimentally alter the release of the active ingredient(s) should be used to form solid oral dosage forms of the SGK1 modulators, such as the SGK1 inhibitors or the SGK1 activators described herein. The amount of disintegrant used varies based upon the type of formulation, and is readily discernible to those of ordinary skill in the art. Disintegrants that can be used to form oral pharmaceutical formulations include, but are not limited to, agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums, and mixtures thereof.

Lubricants that can be used to form oral pharmaceutical formulations of the AhR inhibitors described herein, include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL® 200, manufactured by W. R. Grace Co. of Baltimore, Md.), a coagulated aerosol of synthetic silica (marketed by Degussa Co. of Piano, Tex.), CAB-O-SIL® (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

In other embodiments, lactose-free pharmaceutical formulations and dosage forms are provided, wherein such compositions preferably contain little, if any, lactose or other mono- or di-saccharides. As used herein, the term “lactose-free” means that the amount of lactose present, if any, is insufficient to substantially increase the degradation rate of an active ingredient. Lactose-free compositions of the disclosure can comprise excipients which are well known in the art and are listed in the USP(XXI)/NF (XVI), which is incorporated herein by reference.

The oral formulations of the SGK1 modulators, such as the SGK1 inhibitors or the SGK1 activators described herein, further encompass, in some embodiments, anhydrous pharmaceutical compositions and dosage forms comprising these SGK1 inhibitors or SGK1 activators as active ingredients, since water can facilitate the degradation of some compounds. For example, the addition of water (e.g., 5%) is widely accepted in the pharmaceutical arts as a means of simulating long-term storage in order to determine characteristics such as shelf life or the stability of formulations over time. See, e.g., Jens T. Carstensen, Drug Stability: Principles & Practice, 379-80 (2nd ed., Marcel Dekker, NY, N.Y.: 1995). Anhydrous pharmaceutical compositions and dosage forms described herein can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected. Anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials) with or without desiccants, blister packs, and strip packs.

An SGK1 modulator, such as an SGK1 inhibitor or an SGK1 activator described herein, can be administered directly to the airways in the form of an aerosol or by nebulization. Accordingly, for use as aerosols, in some embodiments, an SGK1 modulator may be packaged in a pressurized aerosol container together with suitable propellants, for example, hydrocarbon propellants like propane, butane, or isobutane with conventional adjuvants. In other embodiments, the SGK1 inhibitor or the SGK1 activator inhibitor can be administered in a non-pressurized form such as in a nebulizer or atomizer.

The term “nebulization” is well known in the art to include reducing liquid to a fine spray. Preferably, by such nebulization small liquid droplets of uniform size are produced from a larger body of liquid in a controlled manner. Nebulization can be achieved by any suitable means, including by using many nebulizers known and marketed today. As is well known, any suitable gas can be used to apply pressure during the nebulization, with preferred gases being those which are chemically inert to the SGK1 modulator, such as the SGK1 inhibitors or the SGK1 activators described herein. Exemplary gases include, but are not limited to, nitrogen, argon or helium.

In other embodiments, an SGK1 modulator, such as an SGK1 inhibitor or an SGK1 activator described herein, can be administered directly to the airways in the form of a dry powder. For use as a dry powder, an SGK1 inhibitor or an SGK1 activator can be administered by use of an inhaler. Exemplary inhalers include metered dose inhalers and dry powdered inhalers.

Suitable powder compositions include, by way of illustration, powdered preparations of an SGK1 modulator, such as an SGK1 inhibitor or an SGK1 activator described herein, thoroughly intermixed with lactose, or other inert powders acceptable for, e.g., intrabronchial administration. The powder compositions can be administered via an aerosol dispenser or encased in a breakable capsule which may be inserted by the subject into a device that punctures the capsule and blows the powder out in a steady stream suitable for inhalation. The compositions can include propellants, surfactants, and co-solvents and may be filled into conventional aerosol containers that are closed by a suitable metering valve.

Aerosols for the delivery to the respiratory tract are known in the art. See for example, Adjei, A. and Garren, J. Pharm. Res., 1: 565-569 (1990); Zanen, P. and Lamm, J.-W. J. Int. J. Pharm., 114: 111-115 (1995); Gonda, I. “Aerosols for delivery of therapeutic an diagnostic agents to the respiratory tract,” in Critical Reviews in Therapeutic Drug Carrier Systems, 6:273-313 (1990); Anderson et al., Am. Rev. Respir. Dis., 140: 1317-1324 (1989)) and have potential for the systemic delivery of peptides and proteins as well (Patton and Platz, Advanced Drug Delivery Reviews, 8:179-196 (1992)); Timsina et. al., Int. J. Pharm., 101: 1-13 (1995); and Tansey, I. P., Spray Technol. Market, 4:26-29 (1994); French, D. L., Edwards, D. A. and Niven, R. W., Aerosol Sci., 27: 769-783 (1996); Visser, J., Powder Technology 58: 1-10 (1989)); Rudt, S, and R. H. Muller, J. Controlled Release, 22: 263-272 (1992); Tabata, Y, and Y. Ikada, Biomed. Mater. Res., 22: 837-858 (1988); Wall, D. A., Drug Delivery, 2: 10 1-20 1995); Patton, J. and Platz, R., Adv. Drug Del. Rev., 8: 179-196 (1992); Bryon, P., Adv. Drug. Del. Rev., 5: 107-132 (1990); Patton, J. S., et al., Controlled Release, 28: 15 79-85 (1994); Damms, B. and Bains, W., Nature Biotechnology (1996); Niven, R. W., et al., Pharm. Res., 12(9); 1343-1349 (1995); and Kobayashi, S., et al., Pharm. Res., 13(1): 80-83 (1996), contents of all of which are herein incorporated by reference in their entirety.

Topical dosage forms of the SGK1 modulators, such as the SGK1 inhibitors or the SGK1 activators described herein, are also provided in some embodiments, and include, but are not limited to, creams, lotions, ointments, gels, shampoos, sprays, aerosols, solutions, emulsions, and other forms known to one of skill in the art. See, e.g., Remington's Pharmaceutical Sciences, 18th ed., Mack Publishing, Easton, Pa. (1990); and Introduction to Pharmaceutical Dosage Forms, 4th ed., Lea & Febiger, Philadelphia, Pa. (1985). For non-sprayable topical dosage forms, viscous to semi-solid or solid forms comprising a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as freon), or in a squeeze bottle. Moisturizers or humectants can also be added to pharmaceutical compositions and dosage forms if desired. Examples of such additional ingredients are well known in the art. See, e.g., Remington's Pharmaceutical Sciences, 18.sup.th Ed., Mack Publishing, Easton, Pa. (1990). and Introduction to Pharmaceutical Dosage Forms, 4th Ed., Lea & Febiger, Philadelphia, Pa. (1985). Dosage forms suitable for treating mucosal tissues within the oral cavity can be formulated as mouthwashes, as oral gels, or as buccal patches. Additional transdermal dosage forms include “reservoir type” or “matrix type” patches, which can be applied to the skin and worn for a specific period of time to permit the penetration of a desired amount of active ingredient.

Examples of transdermal dosage forms and methods of administration that can be used to administer an SGK1 modulator, such as an SGK1 inhibitor or an SGK1 activator described herein, include, but are not limited to, those disclosed in U.S. Pat. Nos. 4,624,665; 4,655,767; 4,687,481; 4,797,284; 4,810,499; 4,834,978; 4,877,618; 4,880,633; 4,917,895; 4,927,687; 4,956,171; 5,035,894; 5,091,186; 5,163,899; 5,232,702; 5,234,690; 5,273,755; 5,273,756; 5,308,625; 5,356,632; 5,358,715; 5,372,579; 5,421,816; 5,466,465; 5,494,680; 5,505,958; 5,554,381; 5,560,922; 5,585,111; 5,656,285; 5,667,798; 5,698,217; 5,741,511; 5,747,783; 5,770,219; 5,814,599; 5,817,332; 5,833,647; 5,879,322; and 5,906,830, each of which are incorporated herein by reference in their entirety.

Suitable excipients (e.g., carriers and diluents) and other materials that can be used to provide transdermal and mucosal dosage forms of the inhibitors described herein are well known to those skilled in the pharmaceutical arts, and depend on the particular tissue or organ to which a given pharmaceutical composition or dosage form will be applied. In addition, depending on the specific tissue to be treated, additional components may be used prior to, in conjunction with, or subsequent to treatment with an SGK1 modulator, such as an SGK1 inhibitor or an SGK1 activator described herein. For example, penetration enhancers can be used to assist in delivering the active ingredients to or across the tissue.

In some embodiments of the aspects described herein, the pharmaceutical formulations comprising the SGK1 modulators, such as the SGK1 inhibitors or the SGK1 activators described herein, can further comprise more than one active compound as necessary for the particular indication being treated, preferably those with complementary activities that do not adversely affect each other. In other embodiments, the formulation comprising the SGK1 modulator, such as an SGK1 inhibitor or an SGK1 activator, can comprise a cytotoxic agent, cytokine, a cytokine inhibitory agent, or a growth inhibitory agent. Such molecules are suitably present in combination in amounts that are effective for the purpose intended.

In some embodiments, the active ingredients of the formulations comprising SGK1 modulators, such as the SGK1 inhibitors or the SGK1 activators described herein, can also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

In some embodiments, the SGK1 modulator, such as the SGK1 inhibitors or the SGK1 activators described herein, can be administered to a subject by controlled- or delayed-release means. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Cherng-ju, Controlled Release Dosage Form Design, 2 (Technomic Publishing, Lancaster, Pa.: 2000)). Controlled-release formulations can be used to control an SGK1 inhibitor's or activator's onset of action, duration of action, plasma levels within the therapeutic window, and peak blood levels. In particular, controlled- or extended-release dosage forms or formulations can be used to ensure that the maximum effectiveness of the SGK1 modulators, such as the SGK1 inhibitors or the SGK1 activators described herein, is achieved while minimizing potential adverse effects and safety concerns, which can occur both from under-dosing a drug (i.e., going below the minimum therapeutic levels) as well as exceeding the toxicity level for the drug.

A variety of known controlled- or extended-release dosage forms, formulations, and devices can be adapted for use with the SGK1 modulators, such as the SGK1 inhibitor or the SGK1 activators described herein. Examples include, but are not limited to, those described in U.S. Pat. Nos. 3,845,770; 3,916,899; 3,536,809; 3,598,123; 4,008,719; 5674,533; 5,059,595; 5,591,767; 5,120,548; 5,073,543; 5,639,476; 5,354,556; 5,733,566; and 6,365,185 Bl; each of which is incorporated ins entirety herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, Duolite® A568 and Duolite® AP143 (Rohm&Haas, Spring House, Pa. USA).

In some embodiments, the SGK1 modulators, such as the SGK1 inhibitors or the SGK1 activator described herein, for use in the various therapeutic formulations and compositions, and methods thereof, are administered to a subject by sustained release or in pulses. Pulse therapy is not a form of discontinuous administration of the same amount of a composition over time, but comprises administration of the same dose of the composition at a reduced frequency or administration of reduced doses. Sustained release or pulse administrations are particularly preferred in chronic conditions, such as autoimmune disorders or chronic inflammatory conditions, as each pulse dose can be reduced and the total amount of a compound of an, e.g., SGK1 inhibitor, such as a small molecule of Formula (I) or Formula (Ia), administered over the course of treatment to the patient is minimized.

The interval between pulses, when necessary, can be determined by one of ordinary skill in the art. Often, the interval between pulses can be calculated by administering another dose of the composition when the composition or the active component of the composition is no longer detectable in the subject prior to delivery of the next pulse. Intervals can also be calculated from the in vivo half-life of the composition. Intervals may be calculated as greater than the in vivo half-life, or 2, 3, 4, 5 and even 10 times greater the composition half-life. Various methods and apparatus for pulsing compositions by infusion or other forms of delivery to the patient are disclosed in U.S. Pat. Nos. 4,747,825; 4,723,958; 4,948,592; 4,965,251 and 5,403,590.

In some embodiments, sustained-release preparations comprising the SGK1 modulator, such as an SGK1 inhibitor or an SGK1 activator described herein, can be prepared. Suitable examples of sustained-release preparations include semipermeable matrices of solid hydrophobic polymers containing the inhibitor, in which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations comprising the SGK1 modulators, such as the SGK1 inhibitors or the SGK1 activators described herein, to be used for in vivo administration are preferably sterile. This is readily accomplished by filtration through, for example, sterile filtration membranes, and other methods known to one of skill in the art.

### Modulating TH17-Mediated Immune Responses by SGK1 Modulation

Certain aspects of the methods described herein are based, in part, on the discovery by the inventors that TH17 differentiation and maintenance of TH17 cells is impaired or inhibited in the absence of SGK1 expression and/or activity. Thus, in some aspects described herein are SGK1 inhibitors for inhibiting TH17 differentiation and activity, and inhibiting TH17-mediated immune responses. In other aspects, described herein are SGK1 activators for promoting or increasing TH17 differentiation and activity, and promoting and increasing TH17-mediated immune responses. Accordingly, the methods using the SGK1 inhibitors and SGK1 activators described herein are useful in the treatment of subjects having diseases or disorders mediated or modulated by TH17 expression and or activity, such as autoimmunity, chronic inflammatory disorders, infectious diseases, cancer, allergic conditions, and the like.

A “TH17-mediated immune response” refers to an immune response that is associated with the induction of, differentiation of, expansion of, proliferation of, functional activity of, or a combination thereof, one or more TH17 cells. At a minimum, as used herein, a “TH17 cell” refers to a CD4+ T cell that expresses and/or produces IL-17A, also known herein as “IL-17.” In some embodiments, a TH17 cell is further characterized by expression of one or more cytokines selected from the following: IL-17F, IL-22, IL-26, IL-21, and TNF-α. In some embodiments, a TH17 cell is further characterized by cell-surface expression of the chemokine receptor CCR6. In some embodiments, a TH17 cell is further characterized by cell-surface expression of the chemokine receptors CCR6 and CCR4. In some embodiments, a TH17 cell is further characterized by cell-surface expression of the chemokine receptor CCR6 and IL23R. In some embodiments, a TH17 cell is further characterized by cell-surface expression of the C-type lectin CD161. In some embodiments, a TH17 cell can be further characterized by expression or activity of one or more of the following factors: RORγt, RORα, STAT3, IRF4, the AhR (aryl hydrocarbon receptor), and BATf. In some embodiments, a TH17 cell can be further characterized as a cell expressing or producing IL-17, but not expressing or producing certain cytokines, such as IL-4, IL-5, and IFN-γ. In some embodiments, a TH17 cell can be further characterized as a cell expressing or producing IL-17, but not expressing or producing certain transcription factors such as T-bet, GATA-3, FOXP3, STAT1, STAT4, and STAT5.

TH17 cells as described herein can be generated or propagated under a variety of conditions. In some embodiments, a TH17 cell can be generated or derived from a naïve CD4 T cell in the presence of TGF-β and IL-6. In some embodiments, a TH17 cell can be generated or derived from a naïve CD4 T cell in the presence of TGF-β and IL-21. In other embodiments, a TH17 cell or a population of TH17 cells is generated or derived from expansion of a population of TH17 cells in the presence of IL-23. In other embodiments, a population of TH17 cells can be maintained in the presence of IL-23.

Accordingly, in some aspects and embodiments described herein, inhibiting a TH17-mediated immune response refers to inhibition of IL-17 expression and/or production by a TH17 cell. In some embodiments, inhibiting a TH17-mediated immune response refers to inhibition of the expression and/or production of IL-17 by a TH17 cell and inhibition of the expression and/or production of one or more of the following cytokines: IL-17F, IL-22, IL-26, IL-21, and TNF-α by a TH17 cell. In such embodiments, inhibition of cytokine production can be assayed using any of a number of methods known to one of skill in the art. For example, biological samples, such as a peripheral blood sample, a serum sample, or a cerebrospinal fluid sample, can be obtained from a subject before and after treatment or administration of an SGK1 modulator as described herein. A statistically significant decrease in the number of cytokine producing cells, as measured, for example, by flow cytometric analysis of TH17 producing cells, is indicative of an inhibition of a TH17 mediated immune response. Cytokine production (and a decrease or inhibition thereof) can also be ascertained in a biological sample, such as serum, using ELISA, bead-based cytokine assays, or any such assay as known to one of skill in the art for measuring cytokine levels.

In some embodiments, inhibiting a TH17-mediated immune response refers to inhibition of proliferation of or expansion of a TH17 cell. In some embodiments, inhibiting a TH17-mediated immune response refers to inhibiting the differentiation of a CD4⁺ T cell or a population of CD4⁺ T cells into a TH17 cell or population of TH17 cells. In such embodiments, changes in proliferation or expansion of a TH17 cell population, or changes in differentiation of a population of a CD4⁺ T cell can be determined using any of a number of assays, including measurement of the number of IL-17 producing cells in biological samples obtained from a subject before and after contacting with, treatment with, or administration of an SGK1 modulator, as described herein. Thus, a reduction in the number of TH17 cells in a contacted, treated, or administered sample relative to a sample prior to such contacting, treating, or administering is indicative of inhibition of the TH17 mediated immune response.

In some embodiments, inhibition of a TH17-mediated immune response refers to inhibition of trafficking of a TH17 cell. Trafficking of a TH17 cell refers to migration of a TH17 cell to a site of immune response activity, such as lymph node during an infection, or a non-lymph node site, such as a joint or the CNS. T cell migration is characterized by changes (up- and down-regulation) in cell-surface marker expression on the T cell. Thus, inhibition of trafficking of a TH17 cell can be measured in biological samples obtained from a subject before and after contacting with, treatment with, or administration of, an SGK1 modulator described herein, by examining expression of cell-surface markers associated with trafficking, such as CD44, CD62L, LFA-1, CCR6, CD73, CCR9, CCR7, and the like, on a TH17 cell or population of cells. In another non-limiting example, TH17 cells in a subject can be labeled using, for example, a dye (e.g., carboxyfluorescein diacetate succinimidyl ester (CFSE) or a dye-antibody conjugate where the antibody recognizes a TH17 cell surface marker) or radioactive moiety, and the movement or accumulation of the cells in the subject can be monitored in the subject before and after contacting with, treatment with, or administration of, an SGK1 modulator as described herein. In addition, in vivo trafficking analyses of TH17 cells can include intravital multiphoton microscopy methods In vivo trafficking of TH17 cells can also be assayed using any of the methods described in T-Cell Trafficking (Methods in Molecular Biology, Vol. 616, Marelli-Berg, F. M. and Nourshargh, Sussan, 1st Edition., 2010, XII, 290), and in Current Protocols in Immunology, Copyright© 2010 by John Wiley and Sons, Inc., the contents of which are also available on the world wide web, the contents of each of which are herein incorporated in their entirety by reference.

Accordingly, in one aspect, described herein are methods for inhibiting differentiation of a precursor CD4⁺ T cell or a CD4⁺ T cell population into a TH17 cell or TH17 cell population. Such methods comprise contacting a CD4⁺ T cell or CD4⁺ T cell population with an inhibitor or antagonist of SGK1 in an amount sufficient to inhibit TH17 cell differentiation. In some embodiments of these methods, the methods of inhibiting TH17 differentiation can further comprise contacting a CD4⁺ T cell or CD4⁺ T cell population with an inhibitor or antagonist of one or more of the following: TGF-β, IL-6, IL-21, IL-23, RORγt, RORα, STAT3, IRF4, the AhR (aryl hydrocarbon receptor), and BATf.

In another aspect, methods of inhibiting a TH17-mediated immune response using an SGK1 inhibitor as described herein are provided. In some embodiments of these methods, inhibiting a TH17-mediated immune response refers to inhibition of IL-17 expression and/or production by a TH17 cell. In some embodiments, inhibiting a TH17-mediated immune response further comprises inhibition of expression and/or production of one or more of the following cytokines: IL-17F, IL-22, IL-26, IL-21, and TNF-α. In some embodiments of these methods, inhibiting a TH17-mediated immune response refers to inhibition of proliferation of or expansion of a TH17 cell. In some embodiments, inhibiting a TH17-mediated immune response refers to inhibition of trafficking of a TH17 cell. Such methods comprise contacting a CD4⁺ T cell or CD4⁺ T cell population with an inhibitor or antagonist of SGK1 in an amount sufficient to inhibit the TH17-mediated immune response. In some embodiments of these methods, the methods of inhibiting a TH17-mediated immune response can further comprise contacting a CD4⁺ T cell or CD4⁺ T cell population with an inhibitor or antagonist of one or more of the following: TGF-β, IL-6, IL-21, IL-23, RORγt, RORα, STAT3, IRF4, the AhR (aryl hydrocarbon receptor), and BATf. In some embodiments of these aspects, the contacting step can be carried out ex vivo, in vitro, or in vivo. For example, in one embodiment, the contacting step is performed using human cells, or performed in a human patient.

By “inhibiting” a TH17-mediated immune response is meant that the production or expression of IL-17 by a TH17 cell or TH17 cell population, the rate of proliferation and/or expansion of a TH17 cell or TH17 cell population, the number of cells differentiating into a TH17 cell, the number or quantity of TH17 cells trafficking to a target tissue or site, or any combination thereof, is at least 10% less, at least 15% less, at least 20% less, at least 25% less, at least 30% less, at least 35% less at least 40% less, at least 45% less, at least 50% less, at least 55% less, at least 60% less, at least 65% less, at least 70% less, at least 75% less, at least 80% less, at least 85% less, at least 90% less, at least 95% less, or completely absent or undetectable in comparison to a reference or control level or sample in the absence of the SGK1 inhibitor.

Accordingly, in one aspect, described herein are methods for inhibiting TH17-mediated immune responses in a subject in need thereof. Such methods comprise administering to a subject in need thereof a therapeutically effective amount of a pharmaceutical composition comprising an inhibitor of SGK1 expression and/or SGK1 activity for inhibiting TH17-mediated immune responses. In some embodiments of these aspects, a subject in need of inhibition of a TH17-mediated immune response has or is at risk for a TH17-mediated disorder. As used herein, a “TH17-mediated disorder” or “TH17-mediated disease” refer to a disease or disorder that is caused by (in part or fully), associated with, or exacerbated by, the development of a TH17-mediated immune response. In such TH17-mediated disorders, inhibition and/or reduction in the TH17-mediated immune response provides a beneficial effect to the subject being treated, i.e., ameliorates, cures, suppresses, delays, prevents the onset of, prevents the recurrence or relapse of one or more of the symptoms associated with the disease or disorder.

The terms “subject” and “individual” are used interchangeably herein, and refer to an animal, for example a human, recipient of the SGK1 inhibitors, such as the small molecules of Formula (I) or Formula (Ia), RNA-based SGK1 inhibitors, or blocking anti-SGK1 antibodies or antigen-binding fragments thereof, or, in other aspects, SGK1 activators described herein. For treatment of those disease states which are specific for a specific animal such as a human subject, the term “subject” refers to that specific animal. The terms ‘non-human animals’ and ‘non-human mammals’ are used interchangeably herein, and include mammals such as rats, mice, rabbits, sheep, cats, dogs, cows, pigs, and non-human primates. The term “subject” also encompasses any vertebrate including but not limited to mammals, reptiles, amphibians and fish. In some embodiments of the aspects described herein, a subject refers to a human subject having an autoimmune disease. In some embodiments of the aspects described herein, a subject refers to a human subject having a chronic inflammatory disease. In some embodiments of the aspects described herein, a subject refers to a human subject having an infectious disease.

In some embodiments of these aspects, the TH17-mediated disease or disorder is an autoimmune disorder. In some embodiments, the methods further comprise selecting or identifying a subject having a TH17-mediated autoimmune disease or disorder.

An “autoimmune disorder” or an “autoimmune disease” as the terms are used herein refer to those disorders or diseases that are the result of inappropriate activation of immune cells that are reactive against self tissue, and which are characterized by the production of cytokines, such as IL-17, and autoantibodies involved in the pathology of the diseases. Preventing the activation or effector function, such as IL-17 production, of autoreactive immune cells can reduce or eliminate disease symptoms. Accordingly, in some embodiments, an autoreactive immune cell is a an autoreactive TH17 cell. Non-limiting examples of autoimmune diseases include multiple sclerosis, rheumatoid arthritis, Crohn's disease, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Grave's disease, autoimmune hemolytic anemia, autoimmune thrombocytopenic purpura, scleroderma (e.g., with anti-collagen antibodies), mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjogren's syndrome, insulin resistance, and autoimmune diabetes mellitus (type 1 diabetes mellitus; insulin-dependent diabetes mellitus). Most autoimmune diseases are also encompassed within the term “chronic inflammatory diseases.” Such diseases or disorders are processes associated with long-term (>6 months) activation of inflammatory immune cells, such as TH17 cells. The chronic inflammation leads to damage of patient organs or tissues. In addition to autoimmune disorders, many diseases are considered to be chronic inflammatory disorders, but are not currently known to have an autoimmune basis. Examples include atherosclerosis, congestive heart failure, polyarteritis nodosa, Whipple's Disease, and primary sclerosing cholangitis.

In another aspect, described herein are methods for promoting TH17-mediated immune responses in a subject in need thereof. Such methods comprise administering to a subject in need thereof a therapeutically effective amount of a pharmaceutical composition comprising an activator of SGK1 expression and/or SGK1 activity for increasing or promoting TH17-mediated immune responses. In such aspects, activating, increasing, or promoting the TH17-mediated immune response provides a beneficial effect to the subject being treated. For example, increasing the TH17-mediated response during an infectious disease or disorder in which expression or production of IL-17 decreases the infectious load or inhibits replication of the infectious agent are non-limiting examples of beneficial effects mediated by a TH17 response.

In some embodiments of these aspects, the TH17-mediated immune response refers to a TH17-mediated infectious immune response. Infectious immune responses in which increased or enhanced TH17 responses are useful include, but are not limited to, responses to extracellular infectious pathogens such as Klebsiella pneumoniae, Staphylococcus aureus, and Candida albicans. In some embodiments, the TH17-mediated immune response refers to a TH17-mediated immune response at an epithelial surface. In some embodiments of these aspects, a TH17-mediated immune response refers to a TH17-mediated mucosal immune response.

In some embodiments, the subject being administered the activator of SGK1 expression and/or SGK1 activity for increasing or promoting TH17-mediated immune responses has a persistent infection with a bacterium, virus, fungus, or parasite. “Persistent infections” refer to those infections that, in contrast to acute infections, are not effectively cleared by the induction of a host immune response. During such persistent infections, the infectious agent and the immune response reach equilibrium such that the infected subject remains infectious over a long period of time without necessarily expressing symptoms. Persistent infections often involve stages of both silent and productive infection without rapidly killing or even producing excessive damage of the host cells. Persistent infections include for example, latent, chronic and slow infections. Persistent infection occurs with viruses including, but not limited to, human T-Cell leukemia viruses, Epstein-Barr virus, cytomegalovirus, herpesviruses, varicella-zoster virus, measles, papovaviruses, prions, hepatitis viruses, adenoviruses, parvoviruses and papillomaviruses.

The mechanisms by which persistent infections are maintained can involve modulation of virus and cellular gene expression and modification of the host immune response. Reactivation of a latent infection can be triggered by various stimuli, including changes in cell physiology, superinfection by another virus, and physical stress or trauma. Host immunosuppression is often associated with reactivation of a number of persistent virus infections.

Additional examples of viral infectious disorders that can be treated using the SGK1 activators described herein include those viral disorders caused by: Retroviridae; Picornaviridae (for example, polio viruses, hepatitis A virus; enteroviruses, human coxsackie viruses, rhinoviruses, echoviruses); Calciviridae (such as strains that cause gastroenteritis); Togaviridae (for example, equine encephalitis viruses, rubella viruses); Flaviridae (for example, dengue viruses, encephalitis viruses, yellow fever viruses); Coronaviridae (for example, coronaviruses); Rhabdoviridae (for example, vesicular stomatitis viruses, rabies viruses); Filoviridae (for example, ebola viruses); Paramyxoviridae (for example, parainfluenza viruses, mumps virus, measles virus, respiratory syncytial virus); Orthomyxoviridae (for example, influenza viruses); Bungaviridae (for example, Hantaan viruses, bunga viruses, phleboviruses and Nairo viruses); Arena viridae (hemorrhagic fever viruses); Reoviridae (e.g., reoviruses, orbiviurses and rotaviruses); Birnaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and HSV-2, varicella zoster virus, cytomegalovirus (CMV), herpes viruses); Poxyiridae (variola viruses, vaccinia viruses, pox viruses); and Iridoviridae (such as African swine fever virus); and unclassified viruses (for example, the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e., Hepatitis C); Norwalk and related viruses, and astroviruses). The compositions and methods described herein are contemplated for use in treating infections with these viral agents.

Additional examples of fungal infections that can be treated using the SGK1 activators described herein include but are not limited to: aspergillosis; thrush (caused by Candida albicans); cryptococcosis (caused by Cryptococcus); and histoplasmosis. Thus, examples of infectious fungi include, but are not limited to, Cryptococcus neoformans, Histoplasma capsulatum, Coccidioides immitis, Blastomyces dermatitidis, Chlamydia trachomatis, Candida albicans. The compositions and methods described herein are contemplated for use in treating infections with these fungal agents.

Further examples of bacterial infections that can be treated using the SGK1 activators described herein include those bacterial disorders caused by: Helicobacter pyloris, Borelia burgdorferi, Legionella pneumophilia, Mycobacteria sps (such as M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracia, corynebacterium diphtheriae, corynebacterium sp., Erysipelothrix rhusiopathiae, Clostridium perfringens, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, and Actinomyces israelli. The compositions and methods described herein are contemplated for use in treating infections with these bacterial agents. Other infectious organisms (such as protists) include: Plasmodium falciparum and Toxoplasma gondii. The compositions and methods described herein are contemplated for use in treating infections with these agents.

In some embodiments of the aspects described herein, the methods further comprise administering an effective amount of a viral, bacterial, fungal, or parasitic antigen in conjunction with the SGK1 activator for promoting TH17 responses. Non-limiting examples of suitable viral antigens include: influenza HA, NA, M, NP and NS antigens; HIV p24, pol, gp41 and gp120; Metapneumovirus (hMNV) F and G proteins; Hepatitis C virus (HCV) E1, E2 and core proteins; Dengue virus (DEN1-4) E1, E2 and core proteins; Human Papilloma Virus L1 protein; Epstein Barr Virus gp220/350 and EBNA-3A peptide; Cytomegalovirus (CMV) gB glycoprotein, gH glycoprotein, pp 65, IE1 (exon 4) and pp 150; Varicella Zoster virus (VZV) 1E62 peptide and glycoprotein E epitopes; Herpes Simplex Virus Glycoprotein D epitopes, among many others. The antigenic polypeptides can correspond to polypeptides of naturally occurring animal or human viral isolates, or can be engineered to incorporate one or more amino acid substitutions as compared to a natural (pathogenic or non-pathogenic) isolate.

In other embodiments of the methods described herein, the subject having a TH17-mediated disorder has a cancer or tumor. A subject that has a cancer or a tumor is a subject having objectively measurable cancer cells present in the subject's body. In some such embodiments of the aspects described herein, the cancer is a solid tumor. Accordingly, provided herein are methods to treat a subject having a cancer or tumor comprising administering a therapeutically effective amount of an SGK1 modulator for modulating TH17-mediated immune responses in the subject having a cancer or a tumor. Depending on the nature of the cancer being treated, in some embodiments, the SGK1 modulator is an SGK1 inhibitor. In some embodiments, the SGK1 modulator is an SGK1 activator.

A “cancer” or “tumor” as used herein refers to an uncontrolled growth of cells which interferes with the normal functioning of the bodily organs and systems. A subject that has a cancer or a tumor is a subject having objectively measurable cancer cells present in the subject's body. Included in this definition are benign and malignant cancers, as well as dormant tumors or micrometastatses. Cancers which migrate from their original location and seed vital organs can eventually lead to the death of the subject through the functional deterioration of the affected organs. Hemopoietic cancers, such as leukemia, are able to out-compete the normal hemopoietic compartments in a subject, thereby leading to hemopoietic failure (in the form of anemia, thrombocytopenia and neutropenia) ultimately causing death.

By “metastasis” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

Metastases are most often detected through the sole or combined use of magnetic resonance imaging (MRI) scans, computed tomography (CT) scans, blood and platelet counts, liver function studies, chest X-rays and bone scans in addition to the monitoring of specific symptoms.

Accordingly, examples of cancer include but are not limited to, carcinoma, lymphoma, blastoma, sarcoma, and leukemia. More particular examples of such cancers include, but are not limited to, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

In some embodiments of these aspects, the methods of treating cancer or a cancerous condition using the SGK1 modulators, such as the SGK1 inhibitors or SGK1 activators described herein, further comprise the step of selecting or identifying a subject having cancer. In such embodiments, a subject is identified as having cancer by objective determination of the presence of cancer cells or a tumor in the subject's body by one of skill in the art. Such objective determinations can be performed through the sole or combined use of tissue biopsies, blood and platelet cell counts, urine analyses, magnetic resonance imaging (MRI) scans, computed tomography (CT) scans, liver function studies, chest X-rays and bone scans in addition to the monitoring of specific symptoms associated with a cancer.

In some embodiments described herein, the methods further comprise administering a tumor or cancer antigen to a subject being administered the SGK1 modulator. A number of tumor antigens have been identified that are associated with specific cancers. As used herein, the terms “tumor antigen” and “cancer antigen” are used interchangeably to refer to antigens which are differentially expressed by cancer cells and can thereby be exploited in order to target cancer cells. Cancer antigens are antigens which can potentially stimulate apparently tumor-specific immune responses. Some of these antigens are encoded, although not necessarily expressed, by normal cells. These antigens can be characterized as those which are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated ras oncogene), suppressor genes (e.g., mutant p53), fusion proteins resulting from internal deletions or chromosomal translocations. Still other cancer antigens can be encoded by viral genes such as those carried on RNA and DNA tumor viruses. Many tumor antigens have been defined in terms of multiple solid tumors: MAGE 1, 2, & 3, defined by immunity; MART-1/Melan-A, gp100, carcinoembryonic antigen (CEA), HER-2, mucins (i.e., MUC-1), prostate-specific antigen (PSA), and prostatic acid phosphatase (PAP). In addition, viral proteins such as hepatitis B (HBV), Epstein-Barr (EBV), and human papilloma (HPV) have been shown to be important in the development of hepatocellular carcinoma, lymphoma, and cervical cancer, respectively. However, due to the immunosuppression of patients diagnosed with cancer, the immune systems of these patients often fail to respond to the tumor antigens.

In some embodiments of the methods described herein, the methods further comprise administering a chemotherapeutic agent to the subject being administered the SGK1 modulator described herein. Non-limiting examples of chemotherapeutic agents can include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb®); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above. In addition, the methods of treatment can further include the use of radiation.

In other embodiments of the methods described herein, the subject in need of modulation of a TH17-mediated immune response has any of the following conditions or disorders: atopic conditions, such as asthma and allergy, including allergic rhinitis, gastrointestinal allergies, including food allergies, eosinophilia, conjunctivitis, In some such embodiments, the SGK1 modulator is an SGK1 inhibitor. In other embodiments, the SGK1 modulator is an SGK1 activator.

### Administration, Dosages, and Durations

An SGK1 modulator, such as the SGK1 inhibitors, e.g., small molecule SGK1 inhibitors of Formula (I) or Formula (Ia) or other small molecule SGK1 inhibitors, RNA-based SGK1 inhibitors, and blocking anti-SGK1 antibodies or antigen-binding fragments described herein, and the SGK1 activators, such as activating anti-SGK1 antibodies and antigen-binding fragments thereof, can be formulated, dosed, and administered in a fashion consistent with good medical practice for use in the treatment of the TH17-mediated disorders described herein, such as autoimmune disorders. Factors for consideration in this context include the particular disorder or type of disorder being treated, the particular subject being treated, the clinical condition of the individual subject, the cause of the disorder, the site of delivery of the agent, the method of administration, the scheduling of administration, and other factors known to medical practitioners.

Accordingly, the “therapeutically effective amount” of an SGK1 modulator, such as the SGK1 inhibitors, e.g., small molecule SGK1 inhibitors of Formula (I) or Formula (Ia), and the SGK1 activators described herein, to be administered is governed by such considerations, and, as used herein, refers to the minimum amount necessary to prevent, ameliorate, or treat, or stabilize, the TH17-mediated disorder. In some embodiments, an SGK1 modulator, is optionally formulated with one or more agents currently used to prevent or treat the disorder being treated. The effective amount of such other agents depends on the amount of the SGK1 modulator present in the formulation, the type of disorder or treatment, and other factors discussed herein, and as understood by one of skill in the art. These are generally used in the same dosages and with administration routes as used herein above or from about 1 to 99% of the heretofore employed dosages.

An effective amount as used herein also includes an amount sufficient to delay the development of a symptom of the TH17-mediated disorder, alter the course of the TH17-mediated disorder (for example but not limited to, inhibit or delay time until relapse in relapsing-remitting multiple sclerosis), or reverse a symptom of the autoimmune disease or disorder. Thus, it is not possible to specify an exact “effective amount”. However, for any given case, an appropriate “effective amount” can be determined by one of ordinary skill in the art using only routine experimentation. If a certain amount of an SGK1 modulator as described herein statistically significantly alters an indicium of a TH17 response, e.g., decreases the number of TH17 cells, reduces the production of IL-17, reduces the proliferation of TH17 cells, and/or reduces trafficking of TH17 cells, as defined herein, it is evidence that said amount is therapeutically effective.

Accordingly, as used herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder associated with a chronic immune condition, such as, but not limited to, an autoimmune disorder, a chronic inflammatory disorder, an infection, or a cancer. Treatment is generally “effective” if one or more symptoms, clinical markers, or indicia of disease are reduced to a clinically significant degree. Alternatively, treatment is “effective” if the progression of a disease is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment).

For example, in some embodiments, the methods described herein comprise administering an effective amount of the SGK1 inhibitors described herein to a subject in order to alleviate one or more symptoms of an autoimmune disorder. As used herein, “alleviating a symptom of an autoimmune disorder” refers to ameliorating any condition or symptom associated with the autoimmune disorder. Alternatively, alleviating a symptom of an autoimmune disorder can involve reducing the number of autoimmune cells in the subject relative to the number of autoimmune cells in an untreated control. In some embodiments, the autoimmune cells comprise TH17 cells. As compared with an equivalent untreated control, such reduction or degree of prevention is at least 5%, 10%, 20%, 40%, 50%, 60%, 80%, 90%, 95%, or 100% as measured by any standard technique. Desirably, the autoimmune disorder is completely abrogated, as detected by any standard method known in the art, in which case the autoimmune disorder is considered to have been cured. A patient who is being treated for an autoimmune disorder is one who a medical practitioner has diagnosed as having such a condition. Diagnosis can be by any suitable means. Diagnosis and monitoring can involve, for example, detecting the level of autoimmune cells or autoantibodies in a biological sample (for example, a tissue biopsy, blood or serum test, or urine test), detecting the level of a surrogate marker of the autoimmune disorder in a biological sample, detecting symptoms associated with the autoimmune disorder, or detecting immune cells involved in the immune response typical of the autoimmune disorder (for example, detection of self-antigen-specific T cells that secrete inflammatory cytokines, such as IL17).

In other embodiments, the methods described herein comprise administering an effective amount of the SGK1 inhibitors or SGK1 activators described herein to a subject in order to alleviate one or more symptoms of a cancer or tumor in a subject in need thereof. As used herein, “alleviating a symptom of a cancer” refers to ameliorating any condition or symptom associated with the cancer. In preferred embodiments, an SGK1 modulator described herein can produce marked anti-cancer effects in a human subject without causing significant toxicities or adverse effects. The efficacy of the SGK1 treatments described herein can be measured by various parameters commonly used in evaluating cancer treatments, including but not limited to, tumor regression, tumor weight or size shrinkage, reduction in rate of tumor growth, the presence or the size of a dormant tumor, the presence or size of metastases or micrometastases, degree of tumor or cancer invasiveness, size or number of the blood vessels, time to progression, duration of survival, progression free survival, overall response rate, duration of response, and quality of life.

Effective amounts, toxicity, and therapeutic efficacy of the SGK1 modulators, such as the SGK1 inhibitors, e.g., small molecule SGK1 inhibitors of Formula (I) or Formula (Ia), and the SGK1 activators described herein, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dosage can vary depending upon the dosage form employed and the route of administration utilized. The dose ratio between toxic and therapeutic effects is the therapeutic index and can be expressed as the ratio LD₅₀/ED₅₀. Compositions and methods that exhibit large therapeutic indices are preferred. A therapeutically effective dose can be estimated initially from cell culture assays. Also, a dose can be formulated in animal models to achieve a circulating plasma concentration range that includes the IC₅₀ (i.e., the concentration of the SGK1 inhibitor or SGK1 activator), which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay. The dosage can be determined by a physician and adjusted, as necessary, to suit observed effects of the treatment.

Depending on the type and severity of the disease, about 1 μg/kg to 100 mg/kg (e.g., 0.1-20 mg/kg) of e.g., a small molecule SGK1 inhibitor of Formula (I) or Formula (Ia) described herein, is an initial candidate dosage range for administration to the subject, whether, for example, by one or more separate administrations, or by continuous infusion. A typical daily dosage might range from about 1 μg/kg to about 100 mg/kg or more, depending on the factors mentioned above. For repeated administrations over several days or longer, depending on the condition, the treatment is sustained until the cancer is treated, as measured by the methods described above or known in the art. However, other dosage regimens may be useful. The progress of the therapeutic methods described herein is easily monitored by conventional techniques and assays, such as those described herein, or known to one of skill in the art. In other embodiments, such dosing regimen is used in combination with a chemotherapy regimen as the first line therapy for treating locally recurrent or metastatic breast cancer.

The duration of the therapeutic methods described herein can continue for as long as medically indicated or until a desired therapeutic effect (e.g., those described herein) is achieved. In certain embodiments, administration of an SGK1 modulator, i.e., “SGK1 inhibitor therapy” or “SGK1 activator therapy” is continued for at least 1 month, at least 2 months, at least 4 months, at least 6 months, at least 8 months, at least 10 months, at least 1 year, at least 2 years, at least 3 years, at least 4 years, at least 5 years, at least 10 years, at least 20 years, or for at least a period of years up to the lifetime of the subject.

The SGK1 modulators described herein, such as the SGK1 inhibitors, e.g., small molecule SGK1 inhibitors of Formula (I) or Formula (Ia), other small molecule SGK1 inhibitors, RNA-based SGK1 inhibitors, and blocking anti-SGK1 antibodies or antigen-binding fragments thereof, and the SGK1 activators, can be administered to a subject, e.g., a human subject, in accordance with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes. Local administration can be used if, for example, extensive side effects or toxicity is associated with the SGK1 inhibitor or the SGK1 activator. An ex vivo strategy can also be used for therapeutic applications.

Exemplary modes of administration of the SGK1 modulators described herein, such as the SGK1 inhibitors, e.g., small molecule SGK1 inhibitors of Formula (I) or Formula (Ia), other small molecule SGK1 inhibitors, RNA-based SGK1 inhibitors, and blocking anti-SGK1 antibodies or antigen-binding fragments thereof, and the SGK1 activators, include, but are not limited to, injection, infusion, inhalation (e.g., intranasal or intratracheal), ingestion, rectal, and topical (including buccal and sublingual) administration. The phrases “parenteral administration” and “administered parenterally” as used herein, refer to modes of administration other than enteral and topical administration, usually by injection. As used herein, “injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. The phrases “systemic administration,” “administered systemically”, “peripheral administration” and “administered peripherally” as used herein refer to the administration of an SGK1 modulator, such as the SGK1 inhibitors, e.g., small molecule SGK1 inhibitors of Formula (I) or Formula (Ia), and the SGK1 activators described herein, other than directly into a target site, tissue, or organ, such as the lung, such that it enters the subject's circulatory system and, thus, is subject to metabolism and other like processes.

In some embodiments, the SGK1 modulators, such as the SGK1 inhibitors, e.g., small molecule SGK1 inhibitors of Formula (I) or Formula (Ia), and the SGK1 activators described herein, are administered by intravenous infusion or injection. In some embodiments, where local treatment is desired, for example, at or near a site of a TH17-mediated immune response, such as in a joint of a patient having rheumatoid arthritis, the SGK1 modulators, such as the SGK1 inhibitors, e.g., small molecule SGK1 inhibitors of Formula (I) or Formula (Ia), and the SGK1 activators can be administered by intralesional administration. Additionally, in some embodiments, the SGK1 inhibitors or SGK1 activators described herein can be administered by pulse infusion, particularly with declining doses of the inhibitors or non-constitutive agonists. Preferably the dosing is given by injections, most preferably intravenous or subcutaneous injections, depending in part on whether the administration is brief or chronic.

The present invention may also be defined in any of the following numbered paragraphs:
1. A method of inhibiting differentiation of a CD4⁺ T cell or a CD4⁺ T cell population into a TH17 cell or TH17 cell population, the method comprising contacting a CD4⁺ T cell or CD4⁺ T cell population with a serum and glucocorticoid-regulated kinase 1 (SGK1) inhibitor in an amount sufficient to inhibit TH17 cell differentiation.
2. The method of paragraph 1, further comprising contacting the CD4⁺ T cell or CD4⁺ T cell population with an inhibitor or antagonist of one or more of the following molecules: TGF-β, IL-6, IL-21, IL-23, RORγt, RORα, STAT3, IRF4, AhR (aryl hydrocarbon receptor), and BATf.
3. A method of inhibiting a TH17 cell-mediated immune response in a subject in need thereof, the method comprising administering to a subject in need thereof a therapeutically effective amount of a serum and glucocorticoid-regulated kinase 1 (SGK1) inhibitor to inhibit a TH17 cell-mediated immune response.
4. The method of paragraph 3, wherein the TH17 cell-mediated response being inhibited comprises expression or production of IL-17 by a TH17 cell.
5. The method of any of paragraphs 3-4, wherein the TH17 cell-mediated response being inhibited comprises expression or production of one or more of IL-17F, IL-22, IL-26, IL-21, and TNF-α.
6. The method of any of paragraphs 3-5, wherein the TH17 cell-mediated response being inhibited comprises inhibition of proliferation of or expansion of a TH17 cell.
7. The method of any of paragraphs 3-6, wherein the TH17 cell-mediated response being inhibited comprises trafficking of a TH17 cell.
8. The method of any of paragraphs 3-7, wherein the subject in need of inhibition of a TH17-mediated immune response has a TH17-mediated disorder.
9. The method of paragraph 8, wherein the TH17-mediated disorder is an autoimmune disease or a chronic inflammatory disease.
10. The method of paragraph 9, wherein the autoimmune disease is multiple sclerosis, rheumatoid arthritis, psoriasis, juvenile rheumatoid arthritis, osteoarthritis, psoriatic arthritis, ankylosing spondylitis, systemic lupus erythematosus, Hashimoto's disease, Graves disease, inflammatory bowel disease, pancreatitis, Crohn's disease, autoimmune diabetes, autoimmune ocular disease, ulcerative colitis, irritable bowel syndrome (IBS), inflammatory bowel disease (IBD), uveitis, or scleritis.
11. The method of any of paragraphs 1-10, wherein the SGK1 inhibitor is a small molecule, a blocking antibody or antigen-binding fragment thereof, a polypeptide, an antisense oligonucleotide, an RNA molecule, an aptamer, or a ribozyme.
12. The method of paragraph 11, wherein the wherein the small molecule is a small molecule of Formula (I):

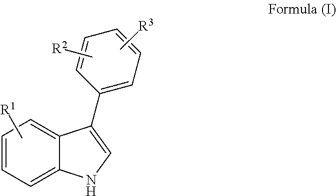

wherein R1 is optionally substituted phenyl, optionally substituted β-napthyl, or optionally substituted 3-CN-phenyl;
wherein R2 is CO₂R4 or C(R4,R5) CO₂R4;
wherein R3 and R4 are independently absent, H, C₁-C₆ alkyl, or C₅-C₈ cycloalkyl; each of which may be optionally substituted;
wherein R5 and R6 are independently absent, H, or C₁-C₆ alkyl, each of which may be optionally substituted; and
pharmaceutically acceptable salts thereof.
13. The method of paragraph 11, wherein the small molecule is a small molecule of Formula (Ia):

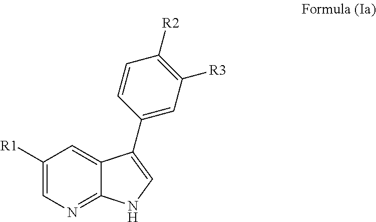

14. The method of paragraph 13, wherein R1 is phenyl, R2 is CO₂H, and R3 is H.
15. The method of paragraph 13, wherein R1 is phenyl, R2 is CO₂H, and R3 is

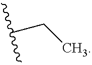 [SMILES: *CC]

16. The method of paragraph 13, wherein R1 is phenyl, R2 is CO₂H, and R3 is

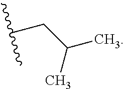 [SMILES: *CC(C)C]

17. The method of paragraph 13, wherein R1 is phenyl, R2 is CO₂H, and R3 is

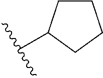 [SMILES: *C1CCCC1]

18. The method of paragraph 13, wherein R1 is β-napthyl, R2 is CH₂CO₂H, and R3 is H.
19. The method of paragraph 13, wherein R1 is β-napthyl, R2 is

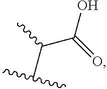 [SMILES: *C(C)C(=O)O]

### and R3 is H.

20. The method of paragraph 13, wherein R1 is β-napthyl, R2 is

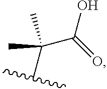

### and R3 is H.

21. The method of paragraph 13, wherein R1 is phenyl, R2 is

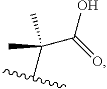

### and R3 is H.

22. The method of paragraph 13, wherein R1 is 3-CN-phenyl, R2 is

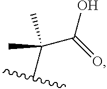

### and R3 is H.

23. The method of paragraph 11, wherein the small molecule is selected from the group consisting of 3-(4-hydroxy-3-methylphenylamino)-4-[(R)-1-(3-hydroxyphenyl)ethylamino]cyclobut-3-ene-1,2-dione; 3-(3-amino-1-tert-butyloxycarbonylindazol-5-ylamino)-4-(3-hydroxybenzylamino)cyclobut-3-ene-1,2-dione; 3-(3-amino-1-tert-butyloxycarbonylindazol-5-ylamino)-4-(3-methoxybenzylamino)cyclobut-3-ene-1,2-dione; 3-(3-amino-1H-indazol-5-ylamino)-4-(3-methoxybenzylamino)cyclobut-3-ene-1,2-dione; 3-(1H-indazol-5-ylamino)-4-[(R)-1-(3-methoxyphenyl)ethylamino]cyclo-but-3-ene-1,2-dione; 3-(1H-indazol-5-ylamino)-4-(3-methoxybenzylamino)cyclobut-3-ene-1,2-dione; 3-(1H-indazol-5-ylamino)-4-(3-hydroxybenzylamino)cyclobut-3-ene-1,2-dione; 3-(1-ethylaminocarbonylindazol-5-ylamino)-4-(3-methoxybenzylamino)cyclobut-3-ene-1,2-dione; 3-(1-ethylaminocarbonylindazol-5-ylamino)-4-(3-hydroxybenzylamino)cyclobut-3-ene-1,2-dione; 3-(3-amino-1H-indazol-5-ylamino)-4-[(R)-1-(3-methoxyphenyl)ethylamino]cyclobut-3-ene-1,2-dione; 3-(3-amino-1H-indazol-5-ylamino)-4-[(R)-1-(3-hydroxyphenyl)ethylamino]cyclobut-3-ene-1,2-dione; 3-(3-amino-1H-indazol-5-ylamino)-4-[(R)-1 (3-chlorophenyl)ethylamino]cyclobut-3-ene-1,2-dione; 3-(3-amino-1H-indazol-5-ylamino)-4-(3-chlorobenzylamino)cyclobut-3-ene-1,2-dione; 3-(3-amino-1H-indazol-5-ylamino)-4-(3-trifluoromethylbenzylamino)cyclobut-3-ene-1,2-dione; 3-(3-amino-1H-indazol-5-ylamino)-4-(3-trifluoromethoxybenzylamino)-cyclobut-3-ene-1,2-dione; 3-(3-amino-1H-indazol-5-ylamino)-4-(3-aminosulfonylbenzylamino)cyclobut-3-ene-1,2-dione; 3-(3-amino-1H-indazol-5-ylamino)-4-[(2-hydroxypyridin-4-ylmethyl)amino]cyclobut-3-ene-1,2-dione; 3-(3-amino-7-methyl-1H-indazol-5-ylamino)-4-[(R)-1-(3-methoxyphenyl)ethylamino]cyclobut-3-ene-1,2-dione; 3-(3-amino-7-methyl-1H-indazol-5-ylamino)-4-[(R)-1-(3-hydroxyphenyl)ethylamino]cyclobut-3-ene-1,2-dione; 3-(3-amino-7-methyl-1H-indazol-5-ylamino)-4-(3-aminosulfonylbenzylamino)cyclobut-3-ene-1,2-dione; 3-(3-amino-7-methyl-1H-indazol-5-ylamino)-4-[(2-hydroxypyridin-4-ylmethyl)amino]cyclobut-3-ene-1,2-dione; 3-(3-amino-7-methyl-1H-indazol-5-ylamino)-4-(3-methoxybenzylamino)-cyclobut-3-ene-1,2-dione; 3-(3-amino-7-methyl-1H-indazol-5-ylamino)-4-(3-hydroxybenzylamino)-cyclobut-3-ene-1,2-dione; 3-[3-(morpholin-4-yl)-1H-indazol-5-ylamino]-4-[(R)-1-(3-hydroxyphe-nyl)ethylamino]cyclobut-3-ene-1,2-dione; 3-[3-(piperidin-1-yl)-1H-indazol-5-ylamino]-4-[(R)-1-(3-hydroxyphenyl)ethylamino]cyclobut-3-ene-1,2-dione; 3-[3-(pyrrolidin-1-yl)-1H-indazol-5-ylamino]-4-[(R)-1-(3-hydroxyphenyl)ethylamino]cyclobut-3-ene-1,2-dione; 3-(3-bromo-1H-indazol-5-ylamino)-4-[(R)-1-(3-hydroxyphenyl)ethylamino]cyclobut-3-ene-1,2-dione; 3-(3-acetamido-1H-indazol-5-ylamino)-4-[(R)-1-(3-hydroxyphenyl)ethylamino]cyclobut-3-ene-1,2-dione; 3-(1H-indazol-5-ylamino)-4-[(R)-1-(3-hydroxyphenyl)ethylamino]cyclobut-3-ene-1,2-dione; 3-(7-bromo-1H-indazol-5-ylamino)-4-(3-methoxybenzylamino)cyclobut-3-ene-1,2-dione; 3-(7-bromo-1H-indazol-5-ylamino)-4-(3-hydroxybenzylamino)cyclobut-3-ene-1,2-dione; 3-(1H-indazol-5-ylamino)-4-(3-chlorobenzylamino)cyclobut-3-ene-1,2-dione; 3-(7-methyl-1H-indazol-5-ylamino)-4-(3-hydroxybenzylamino)cyclobut-3-ene-1,2-dione; 3-(7-methyl-1H-indazol-5-ylamino)-4-[(R)-1-(3-methoxyphenyl)ethylamino]cyclobut-3-ene-1,2-dione; 3-(7-methyl-1H-indazol-5-ylamino)-4-[(S)-1-(3-methoxyphenyl)ethylamino]cyclobut-3-ene-1,2-dione; 3-(7-methyl-1H-indazol-5-ylamino)-4-[(R)-1-(3-hydroxyphenyl)ethylamino]cyclobut-3-ene-1,2-dione; 3-(7-methyl-1H-indazol-5-ylamino)-4-(3-methoxybenzylamino)cyclobut-3-ene-1,2-dione; 3-(1H-indazol-5-ylamino)-4-(3-aminosulfonylbenzylamino)cyclobut-3-ene-1,2-dione; 3-(1H-indazol-5-ylamino)-4-[(R)-1-(3-hydroxyphenyl)-ethylamino]cyclobut-3-ene-1,2-dione; 3-(3-benzoylamino-1H-indazol-5-ylamino)-4-(3-hydroxybenzylamino)cyclobut-3-ene-1,2-dione; 3-(3-benzoylamino-1H-indazol-5-ylamino)-4-(3-methoxybenzylamino)cyclobut-3-ene-1,2-dione; 3-(3-benzoylamino-1H-indazol-5-ylamino)-4-[(R)-1-(3-methoxyphenyl)ethylamino]cyclobut-3-ene-1,2-dione; 3-(3-benzoylamino-1H-indazol-5-ylamino)-4-[(R)-1-(3-hydroxyphenyl)-ethylamino]cyclobut-3-ene-1,2-dione; 3-[3-(3-chlorobenzoylamino)-1H-indazol-5-ylamino]-4-[(R)-1-(3-meth-oxyphenyl)ethylamino]cyclobut-3-ene-1,2-dione; 3-[3-(3-chlorobenzoylamino)-1H-indazol-5-ylamino]-4-[(R)-1-(3-hydroxyphenyl)ethylamino]cyclobut-3-ene-1,2-dione; 3-[3-(3-chlorobenzoylamino)-1H-indazol-5-ylamino]-4-[(R)-1-(3-fluorophenyl)ethylamino]cyclobut-3-ene-1,2-dione; 3-[3-(3-chlorobenzoylamino)-1H-indazol-5-ylamino]-4-[(R)-1-(3-acet-amidophenyl)ethylamino]cyclobut-3-ene-1,2-dione; 3-[3-(3-chlorobenzoylamino)-1H-indazol-5-ylamino]-4-(3-methoxybenzylamino)cyclobut-3-ene-1,2-dione; 3-[3-(3-chlorobenzoylamino)-1H-indazol-5-ylamino]-4-(3-hydroxybenzylamino)cyclobut-3-ene-1,2-dione; 3-[3-(3-chlorobenzoylamino)-1H-indazol-5-ylamino]-4-(3-fluorobenzylamino)cyclobut-3-ene-1,2-dione; 3-[3-(3-chlorobenzoylamino)-1H-indazol-5-ylamino]-4-(3-acetamidobenzylamino)cyclobut-3-ene-1,2-dione; 3-(3-benzoylamino-1H-indazol-5-ylamino)-4-[(R)-1-(2,3-difluorophen-yl)ethylamino]cyclobut-3-ene-1,2-dione; 3-(3-benzoylamino-1H-indazol-5-ylamino)-4-[(R)-1-(3-methylsulfonamidophenyl)ethylamino]cyclobut-3-ene-1,2-dione; 3-[3-(3-chlorobenzoylamino)-1H-indazol-5-ylamino]-4-[(R)-1-(2,3-difluorophenyl)ethylamino]cyclobut-3-ene-1,2-dione; 3-[3-(3-chlorobenzoylamino)-1H-indazol-5-ylamino]-4-[(R)-1-(3-methylsulfonamidophenyl)ethylamino]cyclobut-3-ene-1,2-dione; 3-(3-benzoylamino-1H-indazol-5-ylamino)-4-(2,3-difluorobenzylamino-)cyclobut-3-ene-1,2-dione; 3-(3-benzoylamino-1H-indazol-5-ylamino)-4-(3-methylsulfonamidobenzylamino)cyclobut-3-ene-1,2-dione; 3-[3-(3-chlorobenzoylamino)-1H-indazol-5-ylamino]-4-(2,3-difluorob-enzylamino)cyclobut-3-ene-1,2-dione; and 3-[3-(3-chlorobenzoylamino)-1H-indazol-5-ylamino]-4-(3-methylsulfonamidobenzylamino)cyclobut-3-ene-1,2-dione.
24. The method as in any of paragraphs 3-23, further comprising administering to the subject in need thereof a therapeutic agent selected from the group consisting of a cytokine inhibitor, a growth factor inhibitor, a chemotherapeutic agent, an immunosuppressant, an anti-inflammatory agent, a metabolic inhibitor, an enzyme inhibitor, a cytotoxic agent, and a cytostatic agent.
25. The use of an SGK1 inhibitor in inhibiting a TH17 cell-mediated immune response in a subject in need thereof.
26. The use of the SGK1 inhibitor of paragraph 25, wherein the TH17 cell-mediated response being inhibited comprises expression or production of IL-17 by a TH17 cell.
27. The use of the SGK1 inhibitor of paragraphs 25-26, wherein the TH17 cell-mediated response being inhibited comprises expression or production of one or more of IL-17F, IL-22, IL-26, IL-21, and TNF-α.
28. The use of the SGK1 inhibitor of paragraphs 25-27, wherein the TH17 cell-mediated response being inhibited comprises inhibition of proliferation of or expansion of a TH17 cell.
29. The use of the SGK1 inhibitor of paragraphs 25-28, wherein the TH17 cell-mediated response being inhibited comprises trafficking of a TH17 cell.
30. The use of the SGK1 inhibitor of paragraphs 25-29, wherein the subject in need of inhibition of a TH17-mediated immune response has a TH17-mediated disorder.
31. The use of the SGK1 inhibitor of paragraphs 25-30, wherein the TH17-mediated disorder is an autoimmune disease or a chronic inflammatory disease.
32. The use of the SGK1 inhibitor of paragraph 31, wherein the autoimmune disease is multiple sclerosis, rheumatoid arthritis, psoriasis, juvenile rheumatoid arthritis, osteoarthritis, psoriatic arthritis, ankylosing spondylitis, systemic lupus erythematosus, Hashimoto's disease, Graves disease, inflammatory bowel disease, pancreatitis, Crohn's disease, autoimmune diabetes, autoimmune ocular disease, ulcerative colitis, irritable bowel syndrome (IBS), inflammatory bowel disease (IBD), uveitis, or scleritis.
33. The use of the SGK1 inhibitor of paragraphs 25-32, wherein the SGK1 inhibitor is a small molecule, a blocking antibody or antigen-binding fragment thereof, a polypeptide, an antisense oligonucleotide, an RNA molecule, an aptamer, or a ribozyme.
34. The use of the SGK1 inhibitor of paragraph 33, wherein the small molecule is a small molecule of Formula (I):

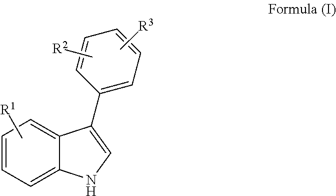

wherein R1 is optionally substituted phenyl, optionally substituted β-napthyl, or optionally substituted 3-CN-phenyl;
wherein R2 is CO₂R4 or C(R4,R5) CO₂R4;
wherein R3 and R4 are independently absent, H, C₁-C₆ alkyl, or C₅-C₈ cycloalkyl; each of which may be optionally substituted;
wherein R5 and R6 are independently absent, H, or C₁-C₆ alkyl, each of which may be optionally substituted; and
pharmaceutically acceptable salts thereof.
35. The use of the SGK1 inhibitor of paragraph 33, wherein the small molecule is a small molecule of Formula (Ia):

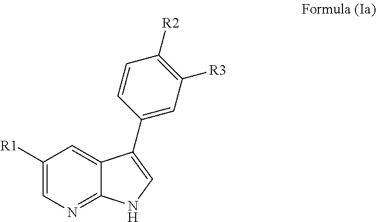

36. The use of the SGK1 inhibitor of paragraph 35, wherein R1 is phenyl, R2 is CO₂H, and R3 is H.
37. The use of the SGK1 inhibitor of paragraph 35, wherein R1 is phenyl, R2 is CO₂, and R3 is

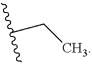 [SMILES: *CC]

38. The use of the SGK1 inhibitor of paragraph 35, wherein R1 is phenyl, R2 is CO₂H, and R3 is

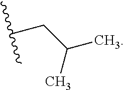 [SMILES: *CC(C)C]

39. The use of the SGK1 inhibitor of paragraph 35, wherein R1 is phenyl, R2 is CO₂H, and R3 is

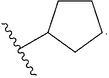 [SMILES: *C1CCCC1]

40. The use of the SGK1 inhibitor of paragraph 35, wherein R1 is β-napthyl, R2 is CH₂CO₂H, and R3 is H.
41. The use of the SGK1 inhibitor of paragraph 35, wherein R1 is β-napthyl, R2 is

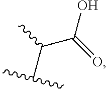 [SMILES: *C(C)C(=O)O]

### and R3 is H.

42. The use of the SGK1 inhibitor of paragraph 35, wherein R1 is β-napthyl, R2 is

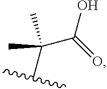

### and R3 is H.

43. The use of the SGK1 inhibitor of paragraph 35, wherein R1 is phenyl, R2 is

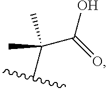

### and R3 is H.

44. The use of the SGK1 inhibitor of paragraph 35, wherein R1 is 3-CN-phenyl, R2 is

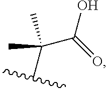

### and R3 is H.

45. The use of the SGK1 inhibitor of paragraph 33, wherein the small molecule is selected from the group consisting of 3-(4-hydroxy-3-methylphenylamino)-4-[(R)-1-(3-hydroxyphenyl)ethylamino]cyclobut-3-ene-1,2-dione; 3-(3-amino-1-tert-butyloxycarbonylindazol-5-ylamino)-4-(3-hydroxybenzylamino)cyclobut-3-ene-1,2-dione; 3-(3-amino-1-tert-butyloxycarbonylindazol-5-ylamino)-4-(3-methoxybenzylamino)cyclobut-3-ene-1,2-dione; 3-(3-amino-1H-indazol-5-ylamino)-4-(3-methoxybenzylamino)cyclobut-3-ene-1,2-dione; 3-(1H-indazol-5-ylamino)-4-[(R)-1-(3-methoxyphenyl)ethylamino]cyclo-but-3-ene-1,2-dione; 3-(1H-indazol-5-ylamino)-4-(3-methoxybenzylamino)cyclobut-3-ene-1,2-dione; 3-(1H-indazol-5-ylamino)-4-(3-hydroxybenzylamino)cyclobut-3-ene-1,2-dione; 3-(1-ethylaminocarbonylindazol-5-ylamino)-4-(3-methoxybenzylamino)cyclobut-3-ene-1,2-dione; 3-(1-ethylaminocarbonylindazol-5-ylamino)-4-(3-hydroxybenzylamino)cyclobut-3-ene-1,2-dione; 3-(3-amino-1H-indazol-5-ylamino)-4-[(R)-1-(3-methoxyphenyl)ethylamino]cyclobut-3-ene-1,2-dione; 3-(3-amino-1H-indazol-5-ylamino)-4-[(R)-1-(3-hydroxyphenyl)ethylamino]cyclobut-3-ene-1,2-dione; 3-(3-amino-1H-indazol-5-ylamino)-4-[(R)-1-(3-chlorophenyl)ethylamino]cyclobut-3-ene-1,2-dione; 3-(3-amino-1H-indazol-5-ylamino)-4-(3-chlorobenzylamino)cyclobut-3-ene-1,2-dione; 3-(3-amino-1H-indazol-5-ylamino)-4-(3-trifluoromethylbenzylamino)cyclobut-3-ene-1,2-dione; 3-(3-amino-1H-indazol-5-ylamino)-4-(3-trifluoromethoxybenzylamino)-cyclobut-3-ene-1,2-dione; 3-(3-amino-1H-indazol-5-ylamino)-4-(3-aminosulfonylbenzylamino)cyclobut-3-ene-1,2-dione; 3-(3-amino-1H-indazol-5-ylamino)-4-[(2-hydroxypyridin-4-ylmethyl)amino]cyclobut-3-ene-1,2-dione; 3-(3-amino-7-methyl-1H-indazol-5-ylamino)-4-[(R)-1-(3-methoxyphenyl)ethylamino]cyclobut-3-ene-1,2-dione; 3-(3-amino-7-methyl-1H-indazol-5-ylamino)-4-[(R)-1-(3-hydroxyphenyl)ethylamino]cyclobut-3-ene-1,2-dione; 3-(3-amino-7-methyl-1H-indazol-5-ylamino)-4-(3-aminosulfonylbenzylamino)cyclobut-3-ene-1,2-dione; 3-(3-amino-7-methyl-1H-indazol-5-ylamino)-4-[(2-hydroxypyridin-4-ylmethyl)amino]cyclobut-3-ene-1,2-dione; 3-(3-amino-7-methyl-1H-indazol-5-ylamino)-4-(3-methoxybenzylamino)-cyclobut-3-ene-1,2-dione; 3-(3-amino-7-methyl-1H-indazol-5-ylamino)-4-(3-hydroxybenzylamino)-cyclobut-3-ene-1,2-dione; 3-[3-(morpholin-4-yl)-1H-indazol-5-ylamino]-4-[(R)-1-(3-hydroxyphe-nyl)ethylamino]cyclobut-3-ene-1,2-dione; 3-[3-(piperidin-1-yl)-1H-indazol-5-ylamino]-4-[(R)-1-(3-hydroxyphenyl)ethylamino]cyclobut-3-ene-1,2-dione; 3-[3-(pyrrolidin-1-yl)-1H-indazol-5-ylamino]-4-[(R)-1-(3-hydroxyphenyl)ethylamino]cyclobut-3-ene-1,2-dione; 3-(3-bromo-1H-indazol-5-ylamino)-4-[(R)-1-(3-hydroxyphenyl)ethylamino]cyclobut-3-ene-1,2-dione; 3-(3-acetamido-1H-indazol-5-ylamino)-4-[(R)-1-(3-hydroxyphenyl)ethylamino]cyclobut-3-ene-1,2-dione; 3-(1H-indazol-5-ylamino)-4-[(R)-1-(3-hydroxyphenyl)ethylamino]cyclobut-3-ene-1,2-dione; 3-(7-bromo-1H-indazol-5-ylamino)-4-(3-methoxybenzylamino)cyclobut-3-ene-1,2-dione; 3-(7-bromo-1H-indazol-5-ylamino)-4-(3-hydroxybenzylamino)cyclobut-3-ene-1,2-dione; 3-(1H-indazol-5-ylamino)-4-(3-chlorobenzylamino)cyclobut-3-ene-1,2-dione; 3-(7-methyl-1H-indazol-5-ylamino)-4-(3-hydroxybenzylamino)cyclobut-3-ene-1,2-dione; 3-(7-methyl-1H-indazol-5-ylamino)-4-[(R)-1-(3-methoxyphenyl)ethylamino]cyclobut-3-ene-1,2-dione; 3-(7-methyl-1H-indazol-5-ylamino)-4-[(S)-1-(3-methoxyphenyl)ethylamino]cyclobut-3-ene-1,2-dione; 3-(7-methyl-1H-indazol-5-ylamino)-4-[(R)-1-(3-hydroxyphenyl)ethylamino]cyclobut-3-ene-1,2-dione; 3-(7-methyl-1H-indazol-5-ylamino)-4-(3-methoxybenzylamino)cyclobut-3-ene-1,2-dione; 3-(1H-indazol-5-ylamino)-4-(3-aminosulfonylbenzylamino)cyclobut-3-ene-1,2-dione; 3-(1H-indazol-5-ylamino)-4-[(R)-1 (3-hydroxyphenyl)-ethylamino]cyclobut-3-ene-1,2-dione; 3-(3-benzoylamino-1H-indazol-5-ylamino)-4-(3-hydroxybenzylamino)cyclobut-3-ene-1,2-dione; 3-(3-benzoylamino-1H-indazol-5-ylamino)-4-(3-methoxybenzylamino)cyclobut-3-ene-1,2-dione; 3-(3-benzoylamino-1H-indazol-5-ylamino)-4-[(R)-1-(3-methoxyphenyl)ethylamino]cyclobut-3-ene-1,2-dione; 3-(3-benzoylamino-1H-indazol-5-ylamino)-4-[(R)-1-(3-hydroxyphenyl)-ethylamino]cyclobut-3-ene-1,2-dione; 3-[3-(3-chlorobenzoylamino)-1H-indazol-5-ylamino]-4-[(R)-1-(3-meth-oxyphenyl)ethylamino]cyclobut-3-ene-1,2-dione; 3-[3-(3-chlorobenzoylamino)-1H-indazol-5-ylamino]-4-[(R)-1-(3-hydroxyphenyl)ethylamino]cyclobut-3-ene-1,2-dione; 3-[3-(3-chlorobenzoylamino)-1H-indazol-5-ylamino]-4-[(R)-1-(3-fluorophenyl)ethylamino]cyclobut-3-ene-1,2-dione; 3-[3-(3-chlorobenzoylamino)-1H-indazol-5-ylamino]-4-[(R)-1-(3-acet-amidophenyl)ethylamino]cyclobut-3-ene-1,2-dione; 3-[3-(3-chlorobenzoylamino)-1H-indazol-5-ylamino]-4-(3-methoxybenzylamino)cyclobut-3-ene-1,2-dione; 3-[3-(3-chlorobenzoylamino)-1H-indazol-5-ylamino]-4-(3-hydroxybenzylamino)cyclobut-3-ene-1,2-dione; 3-[3-(3-chlorobenzoylamino)-1H-indazol-5-ylamino]-4-(3-fluorobenzylamino)cyclobut-3-ene-1,2-dione; 3-[3-(3-chlorobenzoylamino)-1H-indazol-5-ylamino]-4-(3-acetamidobenzylamino)cyclobut-3-ene-1,2-dione; 3-(3-benzoylamino-1H-indazol-5-ylamino)-4-[(R)-1-(2,3-difluorophen-yl)ethylamino]cyclobut-3-ene-1,2-dione; 3-(3-benzoylamino-1H-indazol-5-ylamino)-4-[(R)-1-(3-methylsulfonamidophenyl)ethylamino]cyclobut-3-ene-1,2-dione; 3-[3-(3-chlorobenzoylamino)-1H-indazol-5-ylamino]-4-[(R)-1-(2,3-difluorophenyl)ethylamino]cyclobut-3-ene-1,2-dione; 3-[3-(3-chlorobenzoylamino)-1H-indazol-5-ylamino]-4-[(R)-1-(3-methylsulfonamidophenyl)ethylamino]cyclobut-3-ene-1,2-dione; 3-(3-benzoylamino-1H-indazol-5-ylamino)-4-(2,3-difluorobenzylamino-)cyclobut-3-ene-1,2-dione; 3-(3-benzoylamino-1H-indazol-5-ylamino)-4-(3-methylsulfonamidobenzylamino)cyclobut-3-ene-1,2-dione; 3-[3-(3-chlorobenzoylamino)-1H-indazol-5-ylamino]-4-(2,3-difluorob-enzylamino)cyclobut-3-ene-1,2-dione; and 3-[3-(3-chlorobenzoylamino)-1H-indazol-5-ylamino]-4-(3-methylsulfonamidobenzylamino)cyclobut-3-ene-1,2-dione.

This invention is further illustrated by the following examples which should not be construed as limiting.

### EXAMPLES

Upregulation of SGK1 during TH17 Cell Differentiation

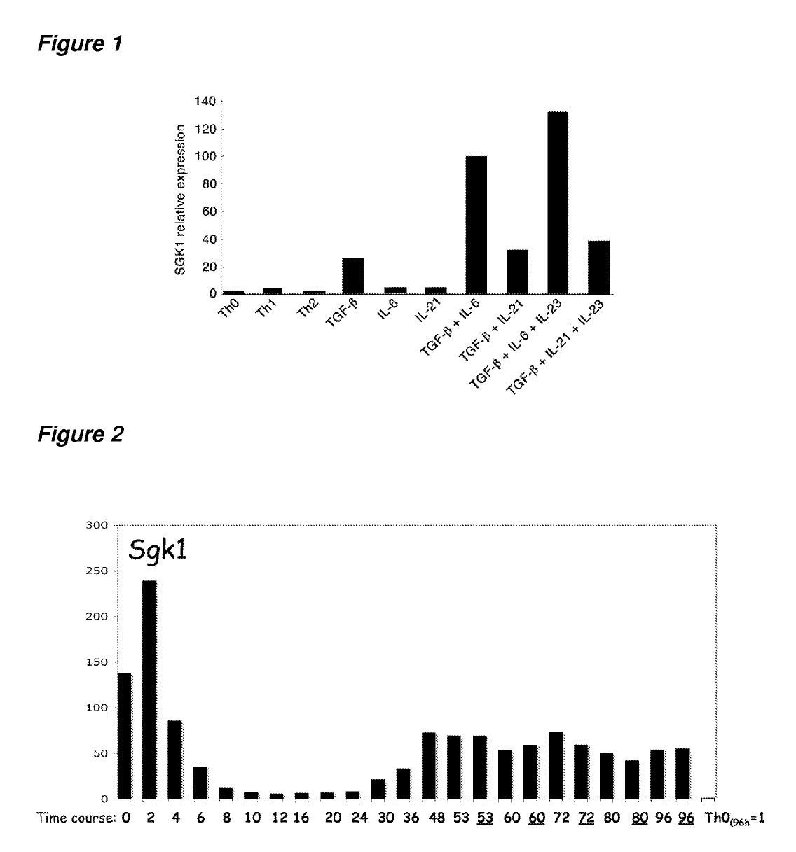

The correlation between the SGK1 expression level during T cell differentiation was previously unknown, so SGK1 mRNA level was first measured under different conditions during T cell differentiation (FIG. 1). The data shown herein clearly demonstrate that TGF-β can upregulate the expression level of SGK1. Moreover, addition of IL-6 together with TGF-β further increased the expression SGK1. IL-23, an IL-12 family cytokine, is essential for enhancing generation of TH17 cells. It was further found that IL-23 further enhances the expression of SGK1 (FIG. 1). These data indicated that SGK1 is specifically induced in TH17 differentiation conditions, and can play a role in differentiation of TH17 cells.

The time dependent expression of SGK1 during TH17 differentiation was further examined by culturing naïve CD4+ T cells under TH17 differentiating conditions (TGF-β plus IL-6) for 96 hours. After 48 hours, TH17 cells were supplemented with IL-23 until the end of the culture period. It was found that expression of SGK1 was rapidly induced after 2 hours and dropped down to baseline levels after 8 hours (FIG. 2). However, IL-23 further induced SGK1 expression (FIG. 2). These results indicate that TH17 differentiating condition (TGF-β plus IL-6) can induce SGK1 expression, which is further sustained and stabilized by IL-23.

### Impaired TH17 Cell Differentiation in Absence of SGK1

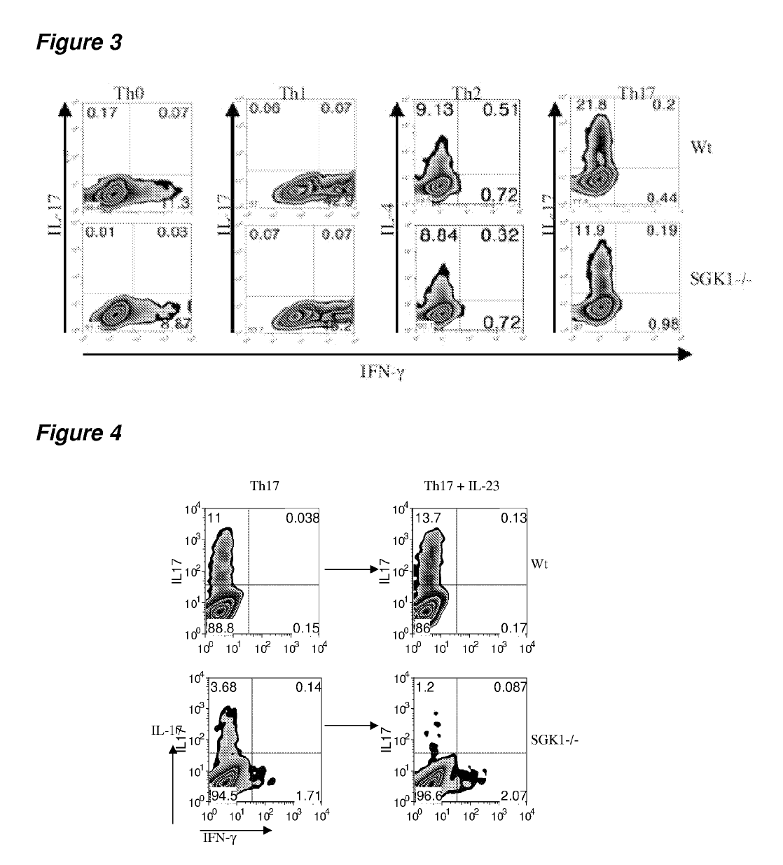

Based on the demonstrations shown herein of regulation of SGK1 expression during TH17 differentiation, Sgk1^{-/-} T cells were tested for the ability to differentiate into TH17 cells. Naïve CD4+ T cells from SGK1 deficient and wild-type mice were differentiated into TH1, TH2 and TH17 cells. It was found that upon stimulation with TGF-β and IL-6, wild-type T cells differentiate into TH17 cell (˜22%). However, SGK-deficient T cells showed significantly reduced IL-17 expression (˜12%) (FIG. 3). However, TH1 and TH2 differentiation did not change in the absence of SGK1, demonstrating the specificity of SGK1 for TH17 differentiation (FIG. 3).

### IL-23 Dependent Maintenance of TH17 Cell was Compromised in Sgk^{-/-} T Cells

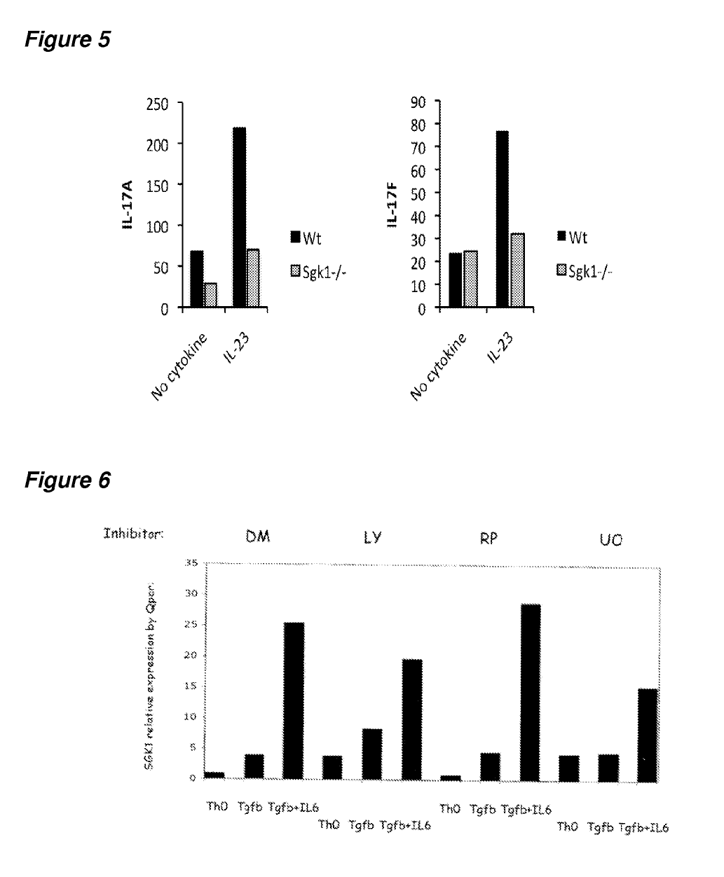

It has been shown that exposure of IL-23 is essential for expanding and stabilizing TH17 cells. The data described herein, as shown in, for example, FIG. 1, clearly demonstrates that IL-23 also induces the expression of SGK1. It was next tested whether SGK1 is essential for IL-23-dependent expansion of TH17 cells. Naïve CD4+ T cells were sorted from wild-type and SGK1-deficient mice and were cultured under TH17 differentiation conditions, as described herein. After a first round of stimulation, cells were rested for two days in a cytokine-free medium. Two days later, cells were activated in the presence or absence of IL-23, and intracellular cytokine staining was performed for IL-17 expression. IL-23 was able to expand already differentiated wild-type TH17 cells (11.5 to 13%), however SGK1-deficient TH17 cells failed to expand in the presence of IL-23 (˜4% to ˜1%). It was further tested whether IL-23 can expand sorted memory TH17 cells. Both wild-type and SGK1-deficient CD4⁺CD62L⁻ cells were sorted and cultured with either anti-CD3 alone or anti-CD3 plus IL-23. IL-23 clearly enhanced the expression of IL-17A and IL-17F in wild-type cells, however SGK1-deficient memory cells were defective in inducing expression of IL-17A and IL-17F (FIG. 5). Altogether, the data shown herein clearly demonstrates that SGK1 is essential for IL-23 dependent expansion of TH17 cells.

### EAE Development in the Absence of SGK1

To study the function of SGK1 in TH-17-mediated autoimmune responses, the susceptibility of wild-type and SGK1-knockout mice to EAE induction can compared. Experimental allergic encephalomyelitis (EAE) is an in vivo model for multiple sclerosis, an autoimmune disease of the brain in humans, the pathology of which has been shown to be mediated by TH17 cells. Mice can be immunized by subcutaneous injection of a peptide consisting of amino acids 35-55 of myelin oligodendrocyte glycoprotein (MOG(35-55)) in complete Freund's adjuvant (CFA) and pertussis toxin. Wild-type mice develop a monophasic disease characterized by ascending paralysis 9-16 d after immunization and prominent leukocyte infiltration and microglial activation in the CNS. MOG-specific T cells that are able to produce IL-17 and IFN-γ are found in the spleens of wild-type mice, and MOG-specific T cells that produce IL-17 and IFN-γ are found in the brains of wild-type diseased mice after 20 days. SGK1 knockout mice can be compared with wild-type mice in terms of the time of onset (kinetics) of paralysis, if any, following immunization; the degree or severity of paralysis, or other disease parameters, if any, following immunization; and the number and degree of infiltration of TH17 cells in the central nervous system (CNS). In mice having impairment of TH17-mediated immune responses, parameters that measure the degree of disease severity in EAE models are found to be inhibited or reduced. The measurement of EAE severity under TH17-inducing conditions is reviewed in “Th17 cells in autoimmune demyelinating disease.” Segal B M. Semin Immunopathol. 2010 March; 32(1):71-77).

We claim:

1. A method of inhibiting differentiation of a CD4⁺ T cell or a CD4⁺ T cell population into a TH17 cell or TH17 cell population, the method comprising contacting a CD4⁺ T cell or CD4⁺ T cell population with a serum and glucocorticoid-regulated kinase 1 (SGK1) inhibitor in an amount sufficient to inhibit TH17 cell differentiation.

2. The method of claim 1, further comprising contacting the CD4⁺ T cell or CD4⁺ T cell population with an inhibitor or antagonist of one or more of the following molecules: TGF-β, IL-6, IL-21, IL-23, RORγt, RORα, STAT3, IRF4, AhR (aryl hydrocarbon receptor), and BATf.

3. A method of inhibiting a TH17 cell-mediated immune response in a subject in need thereof, the method comprising administering to a subject in need thereof a therapeutically effective amount of a serum and glucocorticoid-regulated kinase 1 (SGK1) inhibitor to inhibit a TH17 cell-mediated immune response.

4. The method of claim 3, wherein the TH17 cell-mediated response being inhibited comprises expression or production of IL-17 by a TH17 cell.

5. The method of claim 3, wherein the TH17 cell-mediated response being inhibited comprises expression or production of one or more of IL-17F, IL-22, IL-26, IL-21, and TNF-α.

6. The method of claim 3, wherein the TH17 cell-mediated response being inhibited comprises inhibition of proliferation of or expansion of a TH17 cell.

7. The method of claim 3, wherein the TH17 cell-mediated response being inhibited comprises trafficking of a TH17 cell.

8. The method of claim 3, wherein the subject in need of inhibition of a TH17-mediated immune response has a TH17-mediated disorder.

9. The method of claim 8, wherein the TH17-mediated disorder is an autoimmune disease or a chronic inflammatory disease.

10. The method of claim 9, wherein the autoimmune disease is multiple sclerosis, rheumatoid arthritis, psoriasis, juvenile rheumatoid arthritis, osteoarthritis, psoriatic arthritis, ankylosing spondylitis, systemic lupus erythematosus, Hashimoto's disease, Graves disease, inflammatory bowel disease, pancreatitis, Crohn's disease, autoimmune diabetes, autoimmune ocular disease, ulcerative colitis, irritable bowel syndrome (IBS), inflammatory bowel disease (IBD), uveitis, or scleritis.

11. The method of claim 3, wherein the SGK1 inhibitor is a small molecule, a blocking antibody or antigen-binding fragment thereof, a polypeptide, an antisense oligonucleotide, an RNA molecule, an aptamer, or a ribozyme.

12. The method of claim 11, wherein the small molecule is a small molecule of Formula (I): 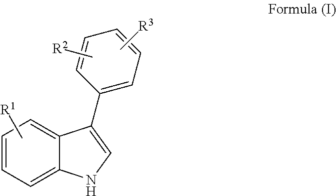
wherein R1 is optionally substituted phenyl, optionally substituted β-napthyl, or optionally substituted 3-CN-phenyl;
wherein R2 is CO₂R4 or C(R4,R5) CO₂R4;
wherein R3 and R4 are independently absent, H, C₁-C₆ alkyl, or C₅-C₈ cycloalkyl; each of which may be optionally substituted;
wherein R5 and R6 are independently absent, H, or C₁-C₆ alkyl, each of which may be optionally substituted; and
pharmaceutically acceptable salts thereof.

13. The method of claim 11, wherein the small molecule is a small molecule of Formula (Ia): 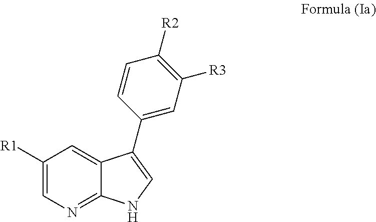
Formula (Ia)

14. The method of claim 13, wherein R1 is phenyl, R2 is CO₂H, and R3 is H.

15. The method of claim 13, wherein R1 is phenyl, R2 is CO₂H, and R3 is

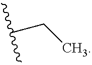 [SMILES: *CC]

16. The method of claim 13, wherein R1 is phenyl, R2 is CO₂H, and R3 is

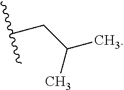 [SMILES: *CC(C)C]

17. The method of claim 13, wherein R1 is phenyl, R2 is CO₂H, and R3 is

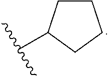 [SMILES: *C1CCCC1]

18. The method of claim 13, wherein R1 is β-napthyl, R2 is CH₂CO₂H, and R3 is H.

19. The method of claim 13, wherein R1 is β-napthyl, R2 is 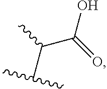 [SMILES: *C(C)C(=O)O]
and R3 is H.

20. The method of claim 13, wherein R1 is β-napthyl, R2 is 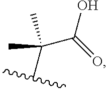
and R3 is H.

21. The method of claim 13, wherein R1 is phenyl, R2 is 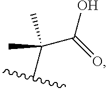
and R3 is H.

22. The method of claim 13, wherein R1 is 3-CN-phenyl, R2 is 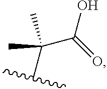
and R3 is H.

23. The method of claim 11, wherein the small molecule is selected from the group consisting of 3-(4-hydroxy-3-methylphenylamino)-4-[(R)-1-(3-hydroxyphenyl)ethylamino]cyclobut-3-ene-1,2-dione; 3-(3-amino-1-tert-butyloxycarbonylindazol-5-ylamino)-4-(3-hydroxybenzylamino)cyclobut-3-ene-1,2-dione; 3-(3-amino-1-tert-butyloxycarbonylindazol-5-ylamino)-4-(3-methoxybenzylamino)cyclobut-3-ene-1,2-dione; 3-(3-amino-1H-indazol-5-ylamino)-4-(3-methoxybenzylamino)cyclobut-3-ene-1,2-dione; 3-(1H-indazol-5-ylamino)-4-[(R)-1-(3-methoxyphenyl)ethylamino]cyclo-but-3-ene-1,2-dione; 3-(1H-indazol-5-ylamino)-4-(3-methoxybenzylamino)cyclobut-3-ene-1,2-dione; 3-(1H-indazol-5-ylamino)-4-(3-hydroxybenzylamino)cyclobut-3-ene-1,2-dione; 3-(1-ethylaminocarbonylindazol-5-ylamino)-4-(3-methoxybenzylamino)cyclobut-3-ene-1,2-dione; 3-(1-ethylaminocarbonylindazol-5-ylamino)-4-(3-hydroxybenzylamino)cyclobut-3-ene-1,2-dione; 3-(3-amino-1H-indazol-5-ylamino)-4-[(R)-1-(3-methoxyphenyl)ethylamino]cyclobut-3-ene-1,2-dione; 3-(3-amino-1H-indazol-5-ylamino)-4-[(R)-1-(3-hydroxyphenyl)ethylamino]cyclobut-3-ene-1,2-dione; 3-(3-amino-1H-indazol-5-ylamino)-4-[(R)-1-(3-chlorophenyl)ethylamino]cyclobut-3-ene-1,2-dione; 3-(3-amino-1H-indazol-5-ylamino)-4-(3-chlorobenzylamino)cyclobut-3-ene-1,2-dione; 3-(3-amino-1H-indazol-5-ylamino)-4-(3-trifluoromethylbenzylamino)cyclobut-3-ene-1,2-dione; 3-(3-amino-1H-indazol-5-ylamino)-4-(3-trifluoromethoxybenzylamino)-cyclobut-3-ene-1,2-dione; 3-(3-amino-1H-indazol-5-ylamino)-4-(3-aminosulfonylbenzylamino)cyclobut-3-ene-1,2-dione; 3-(3-amino-1H-indazol-5-ylamino)-4-[(2-hydroxypyridin-4-ylmethyl)amino]cyclobut-3-ene-1,2-dione; 3-(3-amino-7-methyl-1H-indazol-5-ylamino)-4-[(R)-1-(3-methoxyphenyl)ethylamino]cyclobut-3-ene-1,2-dione; 3-(3-amino-7-methyl-1H-indazol-5-ylamino)-4-[(R)-1-(3-hydroxyphenyl)ethylamino]cyclobut-3-ene-1,2-dione; 3-(3-amino-7-methyl-1H-indazol-5-ylamino)-4-(3-aminosulfonylbenzylamino)cyclobut-3-ene-1,2-dione; 3-(3-amino-7-methyl-1H-indazol-5-ylamino)-4-[(2-hydroxypyridin-4-ylmethyl)amino]cyclobut-3-ene-1,2-dione; 3-(3-amino-7-methyl-1H-indazol-5-ylamino)-4-(3-methoxybenzylamino)-cyclobut-3-ene-1,2-dione; 3-(3-amino-7-methyl-1H-indazol-5-ylamino)-4-(3-hydroxybenzylamino)-cyclobut-3-ene-1,2-dione; 3-[3-(morpholin-4-yl)-1H-indazol-5-ylamino]-4-[(R)-1-(3-hydroxyphenyl)ethylamino]cyclobut-3-ene-1,2-dione; 3-[3-(piperidin-1-yl)-1H-indazol-5-ylamino]-4-[(R)-1-(3-hydroxyphe-nyl)ethylamino]cyclobut-3-ene-1,2-dione; 3-[3-(pyrrolidin-1-yl)-1H-indazol-5-ylamino]-4-[(R)-1-(3-hydroxyph-enyl)ethylamino]cyclobut-3-ene-1,2-dione; 3-(3-bromo-1H-indazol-5-ylamino)-4-[(R)-1-(3-hydroxyphenyl)ethylamino]cyclobut-3-ene-1,2-dione; 3-(3-acetamido-1H-indazol-5-ylamino)-4-[(R)-1-(3-hydroxyphenyl)ethylamino]cyclobut-3-ene-1,2-dione; 3-(1H-indazol-5-ylamino)-4-[(R)-1-(3-hydroxyphenyl)ethylamino]cyclobut-3-ene-1,2-dione; 3-(7-bromo-1H-indazol-5-ylamino)-4-(3-methoxybenzylamino)cyclobut-3-ene-1,2-dione; 3-(7-bromo-1H-indazol-5-ylamino)-4-(3-hydroxybenzylamino)cyclobut-3-ene-1,2-dione; 3-(1H-indazol-5-ylamino)-4-(3-chlorobenzylamino)cyclobut-3-ene-1,2-dione; 3-(7-methyl-1H-indazol-5-ylamino)-4-(3-hydroxybenzylamino)cyclobut-3-ene-1,2-dione; 3-(7-methyl-1H-indazol-5-ylamino)-4-[(R)-1-(3-methoxyphenyl)ethylamino]cyclobut-3-ene-1,2-dione; 3-(7-methyl-1H-indazol-5-ylamino)-4-[(S)-1-(3-methoxyphenyl)ethylamino]cyclobut-3-ene-1,2-dione; 3-(7-methyl-1H-indazol-5-ylamino)-4-[(R)-1-(3-hydroxyphenyl)ethylamino]cyclobut-3-ene-1,2-dione; 3-(7-methyl-1H-indazol-5-ylamino)-4-(3-methoxybenzylamino)cyclobut-3-ene-1,2-dione; 3-(1H-indazol-5-ylamino)-4-(3-aminosulfonylbenzylamino)cyclobut-3-ene-1,2-dione; 3-(1H-indazol-5-ylamino)-4-[(R)-1-(3-hydroxyphenyl)ethylamino]cyclobut-3-ene-1,2-dione; 3-(3-benzoylamino-1H-indazol-5-ylamino)-4-(3-hydroxybenzylamino)cyclobut-3-ene-1,2-dione; 3-(3-benzoylamino-1H-indazol-5-ylamino)-4-(3-methoxybenzylamino)cyclobut-3-ene-1,2-dione; 3-(3-benzoylamino-1H-indazol-5-ylamino)-4-[(R)-1-(3-methoxyphenyl)-ethylamino]cyclobut-3-ene-1,2-dione; 3-(3-benzoylamino-1H-indazol-5-ylamino)-4-[(R)-1-(3-hydroxyphenyl)-ethylamino]cyclobut-3-ene-1,2-dione; 3-[3-(3-chlorobenzoylamino)-1H-indazol-5-ylamino]-4-[(R)-1-(3-meth-oxyphenyl)ethylamino]cyclobut-3-ene-1,2-dione; 3-[3-(3-chlorobenzoylamino)-1H-indazol-5-ylamino]-4-[(R)-1-(3-hydro-oxyphenyl)ethylamino]cyclobut-3-ene-1,2-dione; 3-[3-(3-chlorobenzoylamino)-1H-indazol-5-ylamino]-4-[(R)-1-(3-fluorophenyl)ethylamino]cyclobut-3-ene-1,2-dione; 3-[3-(3-chlorobenzoylamino)-1H-indazol-5-ylamino]-4-[(R)-1-(3-acet-amidophenyl)ethylamino]cyclobut-3-ene-1,2-dione; 3-[3-(3-chlorobenzoylamino)-1H-indazol-5-ylamino]-4-(3-methoxybenz-ylamino)cyclobut-3-ene-1,2-dione; 3-[3-(3-chlorobenzoylamino)-1H-indazol-5-ylamino]-4-(3-hydroxybenz-ylamino)cyclobut-3-ene-1,2-dione; 3-[3-(3-chlorobenzoylamino)-1H-indazol-5-ylamino]-4-(3-fluorobenzylamino)cyclobut-3-ene-1,2-dione; 3-[3-(3-chlorobenzoylamino)-1H-indazol-5-ylamino]-4-(3-acetamidobenzylamino)cyclobut-3-ene-1,2-dione; 3-(3-benzoylamino-1H-indazol-5-ylamino)-4-[(R)-1-(2,3-difluorophen-yl)ethylamino]cyclobut-3-ene-1,2-dione; 3-(3-benzoylamino-1H-indazol-5-ylamino)-4-[(R)-1-(3-methylsulfonamidophenyl)ethylamino]cyclobut-3-ene-1,2-dione; 3-[3-(3-chlorobenzoylamino)-1H-indazol-5-ylamino]-4-[(R)-1-(2,3-di-fluorophenyl)ethylamino]cyclobut-3-ene-1,2-dione; 3-[3-(3-chlorobenzoylamino)-1H-indazol-5-ylamino]-4-[(R)-1-(3-methylsulfonamidophenyl)ethylamino]cyclobut-3-ene-1,2-dione; 3-(3-benzoylamino-1H-indazol-5-ylamino)-4-(2,3-difluorobenzylamino-)cyclobut-3-ene-1,2-dione; 3-(3-benzoylamino-1H-indazol-5-ylamino)-4-(3-methylsulfonamidobenzylamino)cyclobut-3-ene-1,2-dione; 3-[3-(3-chlorobenzoylamino)-1H-indazol-5-ylamino]-4-(2,3-difluorob-enzylamino)cyclobut-3-ene-1,2-dione; and 3-[3-(3-chlorobenzoylamino)-1H-indazol-5-ylamino]-4-(3-methylsulfonamidobenzylamino)cyclobut-3-ene-1,2-dione.

24. The method of claim 3, further comprising administering to the subject in need thereof a therapeutic agent selected from the group consisting of a cytokine inhibitor, a growth factor inhibitor, a chemotherapeutic agent, an immunosuppressant, an anti-inflammatory agent, a metabolic inhibitor, an enzyme inhibitor, a cytotoxic agent, and a cytostatic agent.

25.-45. (canceled)


SEQUENCE LISTING (6 sequences)

SEQ ID NO: 1 - protein, 431 aa, Homo sapiens
MTVKTEAAKGTLTYSRMRGMVAILIAFMKQRRMGLNDFIQKIANNSYACKHPEVQSILKISQPQEPELMNANPSPPPSPSQQINLGPSSNPHAKPSDFHFLKVIGKGSFGKVLLARHKAEEVFYAVKVLQKKAILKKKEEKHIMSERNVLLKNVKHPFLVGLHFSFQTADKLYFVLDYINGGELFYHLQRERCFLEPRARFYAAEIASALGYLHSLNIVYRDLKPENILLDSQGHIVLTDFGLCKENIEHNSTTSTFCGTPEYLAPEVLHKQPYDRTVDWWCLGAVLYEMLYGLPPFYSRNTAEMYDNILNKPLQLKPNITNSARHLLEGLLQKDRTKRLGAKDDFMEIKSHVFFSLINWDDLINKKITPPFNPNVSGPNDLRHFDPEFTEEPVPNSIGKSPDSVLVTASVKEAAEAFLGFSYAPPTDSFL

SEQ ID NO: 2 - protein, 526 aa, Homo sapiens
MVNKDMNGFPVKKCSAFQFFKKRVRRWIKSPMVSVDKHQSPSLKYTGSSMVHIPPGEPDFESSLCQTCLGEHAFQRGVLPQENESCSWETQSGCEVREPCNHANILTKPDPRTFWTNDDPAFMKQRRMGLNDFIQKIANNSYACKHPEVQSILKISQPQEPELMNANPSPPPSPSQQINLGPSSNPHAKPSDFHFLKVIGKGSFGKVLLARHKAEEVFYAVKVLQKKAILKKKEEKHIMSERNVLLKNVKHPFLVGLHFSFQTADKLYFVLDYINGGELFYHLQRERCFLEPRARFYAAEIASALGYLHSLNIVYRDLKPENILLDSQGHIVLTDFGLCKENIEHNSTTSTFCGTPEYLAPEVLHKQPYDRTVDWWCLGAVLYEMLYGLPPFYSRNTAEMYDNILNKPLQLKPNITNSARHLLEGLLQKDRTKRLGAKDDFMEIKSHVFFSLINWDDLINKKITPPFNPNVSGPNDLRHFDPEFTEEPVPNSIGKSPDSVLVTASVKEAAEAFLGFSYAPPTDSFL

SEQ ID NO: 3 - protein, 459 aa, Homo sapiens
MSSQSSSLSEACSREAYSSHNWALPPASRSNPQPAYPWATRRMKEEAIKPPLKAFMKQRRMGLNDFIQKIANNSYACKHPEVQSILKISQPQEPELMNANPSPPPSPSQQINLGPSSNPHAKPSDFHFLKVIGKGSFGKVLLARHKAEEVFYAVKVLQKKAILKKKEEKHIMSERNVLLKNVKHPFLVGLHFSFQTADKLYFVLDYINGGELFYHLQRERCFLEPRARFYAAEIASALGYLHSLNIVYRDLKPENILLDSQGHIVLTDFGLCKENIEHNSTTSTFCGTPEYLAPEVLHKQPYDRTVDWWCLGAVLYEMLYGLPPFYSRNTAEMYDNILNKPLQLKPNITNSARHLLEGLLQKDRTKRLGAKDDFMEIKSHVFFSLINWDDLINKKITPPFNPNVSGPNDLRHFDPEFTEEPVPNSIGKSPDSVLVTASVKEAAEAFLGFSYAPPTDSFL

SEQ ID NO: 4 - protein, 445 aa, Homo sapiens
MGEMQGALARARLESLLRPRHKKRAEAQKRSESFLLSGLAFMKQRRMGLNDFIQKIANNSYACKHPEVQSILKISQPQEPELMNANPSPPPSPSQQINLGPSSNPHAKPSDFHFLKVIGKGSFGKVLLARHKAEEVFYAVKVLQKKAILKKKEEKHIMSERNVLLKNVKHPFLVGLHFSFQTADKLYFVLDYINGGELFYHLQRERCFLEPRARFYAAEIASALGYLHSLNIVYRDLKPENILLDSQGHIVLTDFGLCKENIEHNSTTSTFCGTPEYLAPEVLHKQPYDRTVDWWCLGAVLYEMLYGLPPFYSRNTAEMYDNILNKPLQLKPNITNSARHLLEGLLQKDRTKRLGAKDDFMEIKSHVFFSLINWDDLINKKITPPFNPNVSGPNDLRHFDPEFTEEPVPNSIGKSPDSVLVTASVKEAAEAFLGFSYAPPTDSFL

SEQ ID NO: 5 - RNA, 21 nt, Artificial Sequence
  source: /note=“Description of Artificial Sequence Synthetic oligonucleotide”
guccuucucagcaaaucaauu

SEQ ID NO: 6 - RNA, 21 nt, Artificial Sequence
  source: /note=“Description of Artificial Sequence Synthetic oligonucleotide”
uugauuugcugagaaggacuu